SEQUENCE LISTING (134 sequences)

SEQ ID NO: 1 - protein, 11 aa, synthetic construct
KIGDFGLATVK

SEQ ID NO: 2 - protein, 11 aa, synthetic construct
KIGDFGLATEK

SEQ ID NO: 3 - protein, 10 aa, synthetic construct
KLVVVGAGGV

SEQ ID NO: 4 - protein, 10 aa, synthetic construct
KLVVVGADGV

SEQ ID NO: 5 - skipped

SEQ ID NO: 6 - protein, 10 aa, synthetic construct
KLVVVGAVGV

SEQ ID NO: 7 - protein, 8 aa, synthetic construct
KLVVVGAG

SEQ ID NO: 8 - protein, 8 aa, synthetic construct
KLVVVGAV

SEQ ID NO: 9 - protein, 11 aa, synthetic construct
KIGDFGLATVK

SEQ ID NO: 10 - protein, 11 aa, synthetic construct
KIGDFGLATEK

SEQ ID NO: 11 - protein, 10 aa, synthetic construct
AHHGGWTTKM

SEQ ID NO: 12 - protein, 10 aa, synthetic construct
ARHGGWTTKM

SEQ ID NO: 13-14 - skipped (2 entries)

SEQ ID NO: 15 - protein, 11 aa, synthetic construct
STRDPLSEITE

SEQ ID NO: 16 - protein, 11 aa, synthetic construct
STRDPLSEITK

SEQ ID NO: 17 - protein, 10 aa, synthetic construct
FGLATVKSRW

SEQ ID NO: 18 - protein, 10 aa, synthetic construct
FGLATEKSRW

SEQ ID NO: 19 - protein, 8 aa, synthetic construct
LATVKSRW

SEQ ID NO: 20 - protein, 8 aa, synthetic construct
LATEKSRW

SEQ ID NO: 21 - DNA, 28 bp, synthetic construct
  source 1..28: mol_type = other DNA
ctcgcgctactctctctttctggcctgg

SEQ ID NO: 22 - DNA, 129 bp, synthetic construct
  source 1..129: mol_type = other DNA
gccttagctgtgctcgcgctactctctctttctggcctggaggctatccagcgtgagtctctcctaccctcccgctctggtccttcctctccgctctgcaccctctgtggccctcgctgtgctctctcg

SEQ ID NO: 23 - DNA, 130 bp, synthetic construct
  source 1..130: mol_type = other DNA
cggaatcgacacgagcgcgatgagagagaaagaccggacctcccgataggtcgcactcagagaggatgggagggcgagaccaggaaggagaggcgagacgtgggagacaccgggagcgacacgagagagc

SEQ ID NO: 24 - DNA, 28 bp, synthetic construct
  source 1..28: mol_type = other DNA
gagcgcgatgagagagaaagaccggacc

SEQ ID NO: 25 - protein, 17 aa, synthetic construct
ALAVLALLSLSGLEAIQ

SEQ ID NO: 26 - DNA, 23 bp, synthetic construct
  source 1..23: mol_type = other DNA
gctactctctctttctggccngg

SEQ ID NO: 27 - DNA, 23 bp, synthetic construct
  source 1..23: mol_type = other DNA
actctctctttctggcctggngg

SEQ ID NO: 28 - DNA, 24 bp, synthetic construct
  source 1..24: mol_type = other DNA
ctactctctctttctggcctngag

SEQ ID NO: 29 - DNA, 24 bp, synthetic construct
  source 1..24: mol_type = other DNA
gccagaaagagagagtagcgngag

SEQ ID NO: 30 - DNA, 23 bp, synthetic construct
  source 1..23: mol_type = other DNA
ggacatctggctaaggaactngg

SEQ ID NO: 31 - DNA, 23 bp, synthetic construct
  source 1..23: mol_type = other DNA
ccagaggagcttccggcagacct

SEQ ID NO: 32 - DNA, 131 bp, synthetic construct
  source 1..131: mol_type = other DNA
ctgcagccttcccagaggagcttccggcagacctgaagcactggaagccaggtgtgcagggcaggtgggctggggttgggaagggtggatgccttggggaggggatggaagagatttgaactcctggccca

SEQ ID NO: 33 - DNA, 130 bp, synthetic construct
  source 1..130: mol_type = other DNA
gacgtcggaagggtctcctcgaaggccgtctggacttcgtgaccttcggtccacacgtcccgtccacccgaccccaacccttcccacctacggaaccctcccctaccttctctaaacttgaggaccgggt

SEQ ID NO: 34 - DNA, 23 bp, synthetic construct
  source 1..23: mol_type = other DNA
ggtctcctcgaaggccgtctgga

SEQ ID NO: 35 - protein, 15 aa, synthetic construct
PFPEELPADLKHYKP

SEQ ID NO: 36 - DNA, 23 bp, synthetic construct
  source 1..23: mol_type = other DNA
ccagaggagcttccggcagacct

SEQ ID NO: 37 - DNA, 23 bp, synthetic construct
  source 1..23: mol_type = other DNA
ggtctgccggaagctcctctngg

SEQ ID NO: 38 - DNA, 23 bp, synthetic construct
  source 1..23: mol_type = other DNA
gacctgaagcactggaagccngg

SEQ ID NO: 39 - DNA, 24 bp, synthetic construct
  source 1..24: mol_type = other DNA
ttcccagaggagcttccggcngac

SEQ ID NO: 40 - DNA, 24 bp, synthetic construct
  source 1..24: mol_type = other DNA
agaggagcttccggcagaccngaa

SEQ ID NO: 41 - DNA, 24 bp, synthetic construct
  source 1..24: mol_type = other DNA
tccggcagacctgaagcactngaa

SEQ ID NO: 42 - DNA, 23 bp, synthetic construct
  source 1..23: mol_type = other DNA
tgccggaagctcctctgggangg

SEQ ID NO: 43 - DNA, 28 bp, synthetic construct
  source 1..28: mol_type = other DNA
cgagatgtctcgctccgtggccttagct

SEQ ID NO: 44 - DNA, 28 bp, synthetic construct
  source 1..28: mol_type = other DNA
gctctacagagcgaggcaccggaatcga

SEQ ID NO: 45 - DNA, 26 bp, synthetic construct
  source 1..26: mol_type = other DNA
cgagatgtctcgctccgtggccttag

SEQ ID NO: 46 - DNA, 30 bp, synthetic construct
  source 1..30: mol_type = other DNA
cgagatgtctcgctccgtggccttagctct

SEQ ID NO: 47 - DNA, 23 bp, synthetic construct
  source 1..23: mol_type = other DNA
cgagatgtctcgctccgtggcct

SEQ ID NO: 48 - DNA, 48 bp, synthetic construct
  source 1..48: mol_type = other DNA
gtgctcgcgctactctctctttctggcctggaggctatccagcgtgag

SEQ ID NO: 49 - DNA, 48 bp, synthetic construct
  source 1..48: mol_type = other DNA
cacgagcgcgatgagagagaaagaccggacctccgataggtcgcactc

SEQ ID NO: 50 - DNA, 92 bp, synthetic construct
  source 1..92: mol_type = other DNA
cacatgttttctctgcagccttcccagaggagcttccggcagacctgaagcactggaagccaggtgtgcagggcaggtgggctggggttggg

SEQ ID NO: 51 - DNA, 93 bp, synthetic construct
  source 1..93: mol_type = other DNA
gtgtacaaaagagacgtcggaagggtctcctcgaaggccgtctggacttcgtgaccttcggtccacacgtcccgtcccacccgaccccaaccc

SEQ ID NO: 52 - DNA, 82 bp, synthetic construct
  source 1..82: mol_type = other DNA
cacatgttttctctgcagccttcccagagcagacctgaagcactggaagccaggtgtgcagggcaggtgggctggggttggg

SEQ ID NO: 53 - DNA, 81 bp, synthetic construct
  source 1..81: mol_type = other DNA
cacatgttttctctgcagcagcttccggcagacctgaagcactggaagccaggtgtgcagggcaggtgggctggggttggg

SEQ ID NO: 54 - DNA, 91 bp, synthetic construct
  source 1..91: mol_type = other DNA
cacatgttttctctgcagccttcccagagagcttccggcagacctgaagcactggaagccaggtgtgcagggcaggtgggctggggttggg

SEQ ID NO: 55 - DNA, 79 bp, synthetic construct
  source 1..79: mol_type = other DNA
cacatgttttctctgcagccttccggcagacctgaagcactggaagccaggtgtgcagggcaggtgggctggggttggg

SEQ ID NO: 56 - DNA, 259 bp, synthetic construct
  source 1..259: mol_type = other DNA
tgggttggggcaaagagggaaatgagatcatgtccaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaac

SEQ ID NO: 57 - DNA, 260 bp, synthetic construct
  source 1..260: mol_type = other DNA
acccaaccccgtttctccctttactctagtacaggattgggactaggagaacagggtgtctataggtcttgggactgggacggcacatggtcgactctctgagatttaggtcactgttcagacagacggataagtggctaaaactaagagtttgtttacacagtgtttcattcctaagactacacatatagtgtctgttttgacacgatctgtactccagatacctgaagttctcgttgtcacgacaccggacctcgttg

SEQ ID NO: 58-59 - skipped (2 entries)

SEQ ID NO: 60 - DNA, 23 bp, synthetic construct
  source 1..23: mol_type = other DNA
tcagggttctggatatctgtngg

SEQ ID NO: 61 - DNA, 23 bp, synthetic construct
  source 1..23: mol_type = other DNA
gagaatcaaaatcggtgaatngg

SEQ ID NO: 62 - DNA, 23 bp, synthetic construct
  source 1..23: mol_type = other DNA
tggatttagagtctctcagcngg

SEQ ID NO: 63 - DNA, 23 bp, synthetic construct
  source 1..23: mol_type = other DNA
tctctcagctggtacacggcngg

SEQ ID NO: 64 - DNA, 23 bp, synthetic construct
  source 1..23: mol_type = other DNA
acacggcagggtcagggttcngg

SEQ ID NO: 65 - DNA, 23 bp, synthetic construct
  source 1..23: mol_type = other DNA
gctggtacacggcagggtcangg

SEQ ID NO: 66 - DNA, 23 bp, synthetic construct
  source 1..23: mol_type = other DNA
atctgtgggacaagaggatcngg

SEQ ID NO: 67 - DNA, 23 bp, synthetic construct
  source 1..23: mol_type = other DNA
gtcagggttctggatatctgngg

SEQ ID NO: 68 - DNA, 23 bp, synthetic construct
  source 1..23: mol_type = other DNA
ctctcagctggtacacggcangg

SEQ ID NO: 69 - DNA, 23 bp, synthetic construct
  source 1..23: mol_type = other DNA
ctggatatctgtgggacaagngg

SEQ ID NO: 70 - DNA, 23 bp, synthetic construct
  source 1..23: mol_type = other DNA
agctggtacacggcagggtcngg

SEQ ID NO: 71 - DNA, 23 bp, synthetic construct
  source 1..23: mol_type = other DNA
tctgtgggacaagaggatcangg

SEQ ID NO: 72 - DNA, 23 bp, synthetic construct
  source 1..23: mol_type = other DNA
agagtctctcagctggtacangg

SEQ ID NO: 73 - DNA, 23 bp, synthetic construct
  source 1..23: mol_type = other DNA
atttgtttgagaatcaaaatngg

SEQ ID NO: 74 - DNA, 23 bp, synthetic construct
  source 1..23: mol_type = other DNA
taggcagacagacttgtcacngg

SEQ ID NO: 75 - DNA, 23 bp, synthetic construct
  source 1..23: mol_type = other DNA
gggacaagaggatcagggttngg

SEQ ID NO: 76 - DNA, 130 bp, synthetic construct
  source 1..130: mol_type = other DNA
cctaccagaaccagacagctctcagagcaaccctggctccaacccctcttccctttccagaggacctgaacaaggtgttcccacccgaggtcgctgtgtttgagccatcagaagcagagatctcccacac

SEQ ID NO: 77 - DNA, 130 bp, synthetic construct
  source 1..130: mol_type = other DNA
ggatggtcttggtctgtcgagagtctcgttgggaccgaggttggggagaagggaaaggtctcctggacttgttccacaagggtgggctccagcgacacaaactcggtagtcttcgtctctagagggtgtg

SEQ ID NO: 78 - protein, 23 aa, synthetic construct
DLNKVFPPEVAVFEPSEAEISHT

SEQ ID NO: 79 - DNA, 23 bp, synthetic construct
  source 1..23: mol_type = other DNA
gaacaaggtgttcccacccgngg

SEQ ID NO: 80 - protein, 23 aa, synthetic construct
CTTTCCAGAGGACCTGAACANGG

SEQ ID NO: 81 - DNA, 23 bp, synthetic construct
  source 1..23: mol_type = other DNA
cgggtgggaacaccttgttcngg

SEQ ID NO: 82 - DNA, 23 bp, synthetic construct
  source 1..23: mol_type = other DNA
ggtcctctggaaagggaagangg

SEQ ID NO: 83 - DNA, 23 bp, synthetic construct
  source 1..23: mol_type = other DNA
aacaccttgttcaggtcctcngg

SEQ ID NO: 84 - DNA, 23 bp, synthetic construct
  source 1..23: mol_type = other DNA
cttgttcaggtcctctggaangg

SEQ ID NO: 85 - DNA, 23 bp, synthetic construct
  source 1..23: mol_type = other DNA
gtcctctggaaagggaagagngg

SEQ ID NO: 86 - DNA, 23 bp, synthetic construct
  source 1..23: mol_type = other DNA
caaacacagcgacctcgggtngg

SEQ ID NO: 87 - DNA, 23 bp, synthetic construct
  source 1..23: mol_type = other DNA
tcaaacacagcgacctcgggngg

SEQ ID NO: 88 - DNA, 23 bp, synthetic construct
  source 1..23: mol_type = other DNA
ttgttcaggtcctctggaaangg

SEQ ID NO: 89 - DNA, 23 bp, synthetic construct
  source 1..23: mol_type = other DNA
ggctcaaacacagcgacctcngg

SEQ ID NO: 90 - DNA, 23 bp, synthetic construct
  source 1..23: mol_type = other DNA
aaaggccacactggtgtgccngg

SEQ ID NO: 91 - DNA, 23 bp, synthetic construct
  source 1..23: mol_type = other DNA
acactggtgtgcctggccacngg

SEQ ID NO: 92 - DNA, 23 bp, synthetic construct
  source 1..23: mol_type = other DNA
agagatctcccacacccaaangg

SEQ ID NO: 93 - DNA, 23 bp, synthetic construct
  source 1..23: mol_type = other DNA
aggtcctctggaaagggaagngg

SEQ ID NO: 94 - DNA, 23 bp, synthetic construct
  source 1..23: mol_type = other DNA
tggctcaaacacagcgacctngg

SEQ ID NO: 95 - DNA, 23 bp, synthetic construct
  source 1..23: mol_type = other DNA
ggcacaccagtgtggcctttngg

SEQ ID NO: 96 - DNA, 23 bp, synthetic construct
  source 1..23: mol_type = other DNA
tgggagatctctgcttctgangg

SEQ ID NO: 97 - DNA, 23 bp, synthetic construct
  source 1..23: mol_type = other DNA
cagtgtggccttttgggtgtngg

SEQ ID NO: 98 - DNA, 23 bp, synthetic construct
  source 1..23: mol_type = other DNA
tgtggccaggcacaccagtgngg

SEQ ID NO: 99 - DNA, 23 bp, synthetic construct
  source 1..23: mol_type = other DNA
ccagtgtggccttttgggtgngg

SEQ ID NO: 100 - DNA, 23 bp, synthetic construct
  source 1..23: mol_type = other DNA
ccacacccaaaaggccacacngg

SEQ ID NO: 101 - DNA, 23 bp, synthetic construct
  source 1..23: mol_type = other DNA
gcacaccagtgtggccttttngg

SEQ ID NO: 102 - DNA, 35 bp, synthetic construct
  source 1..35: mol_type = other DNA
gactctaaatccagtgacaagtctgtctgcctatt

SEQ ID NO: 103 - DNA, 35 bp, synthetic construct
  source 1..35: mol_type = other DNA
ctgagatttaggtcactgttcagacagacggataa

SEQ ID NO: 104 - DNA, 22 bp, synthetic construct
  source 1..22: mol_type = other DNA
taaatccagtgacaagtctgtc

SEQ ID NO: 105 - DNA, 52 bp, synthetic construct
  source 1..52: mol_type = other DNA
caccgattttgattctcaaacaaatgtgtcacaaagtaaggattctgatgtg

SEQ ID NO: 106 - DNA, 52 bp, synthetic construct
  source 1..52: mol_type = other DNA
gtggctaaaactaagagtttgtttacacagtgtttcattcctaagactacac

SEQ ID NO: 107 - DNA, 51 bp, synthetic construct
  source 1..51: mol_type = other DNA
accgattttgattctcaaacaaatgtgtcacaaagtaaggattctgatgtg

SEQ ID NO: 108 - DNA, 54 bp, synthetic construct
  source 1..54: mol_type = other DNA
ttcaccgattttgattctcaaacaaatgtgtcacaaagtaaggattctgatgtg

SEQ ID NO: 109 - DNA, 95 bp, synthetic construct
  source 1..95: mol_type = other DNA
ttccctttccagaggacctgaacaaggtgttcccacccgaggtcgctgtgtttgagccatcagaagcagagatctcccacacccaaaaggccaca

SEQ ID NO: 110 - DNA, 94 bp, synthetic construct
  source 1..94: mol_type = other DNA
aagggaaaggtctcctggacttgttccacaagggtgggctccagcgacacaaactcggtagtcttcgtctctagaggtgtgggttttccggtgt

SEQ ID NO: 111 - protein, 27 aa, synthetic construct
KSSDKSVCLFTDFDSQTNVSQSKDSDV

SEQ ID NO: 112 - protein, 27 aa, synthetic construct
DLNKVFPPEVAVFEPSEAEISHTQKAT

SEQ ID NO: 113 - DNA, 75 bp, synthetic construct
  source 1..75: mol_type = other DNA
ttccctttccagaggcccgaggtcgctgtgtttgagccatcagaagcagagatctcccacacccaaaaggccaca

SEQ ID NO: 114 - DNA, 94 bp, synthetic construct
  source 1..94: mol_type = other DNA
ttccctttccagaggacctgaacaaggtgttccgaccgaggtcgctgtgtttgagccatcagaagcagagatctcccacacccaaaaggccaca

SEQ ID NO: 115 - skipped

SEQ ID NO: 116 - DNA, 93 bp, synthetic construct
  source 1..93: mol_type = other DNA
ttccctttccagaggacctgaacaaggtgttcccccgaggtcgctgtgtttgagccatcagaagcagagatctcccacacccaaaaggccaca

SEQ ID NO: 117 - DNA, 130 bp, synthetic construct
  source 1..130: mol_type = other DNA
ctatctcctggcgataaccaattttcccaccatctttcctcttaggagacttgcctggtgaaaatcatcactggtcttttggagtttgaggtatacctagagtacctccagaacagatttgagagtagtg

SEQ ID NO: 118 - DNA, 74 bp, synthetic construct
  source 1..74: mol_type = other DNA
gatagaggaccgctattggttaaaagggtggtagaaaggagaatcctctgaacggaccacttttagtagtgacc

SEQ ID NO: 119 - DNA, 23 bp, synthetic construct
  source 1..23: mol_type = other DNA
cctggtgaaaatcatcactggtc

SEQ ID NO: 120 - DNA, 23 bp, synthetic construct
  source 1..23: mol_type = other DNA
ggaccacttttagtagtgaccag

SEQ ID NO: 121 - protein, 27 aa, synthetic construct
ETCLVKIITGLLFEVYLEYLQNRFESS

SEQ ID NO: 122 - DNA, 23 bp, synthetic construct
  source 1..23: mol_type = other DNA
tgcctggtgaaaatcatcacngg

SEQ ID NO: 123 - DNA, 23 bp, synthetic construct
  source 1..23: mol_type = other DNA
gaccagtgatgattttcaccngg

SEQ ID NO: 124 - DNA, 23 bp, synthetic construct
  source 1..23: mol_type = other DNA
gaaaatcatcactggtctttngg

SEQ ID NO: 125 - DNA, 23 bp, synthetic construct
  source 1..23: mol_type = other DNA
ttcctcttaggagacttgccngg

SEQ ID NO: 126 - DNA, 25 bp, synthetic construct
  source 1..25: mol_type = other DNA
ctcttaggagacttgcctggngaaa

SEQ ID NO: 127 - DNA, 24 bp, synthetic construct
  source 1..24: mol_type = other DNA
ttcaccaggcaagtctcctangag

SEQ ID NO: 128 - DNA, 24 bp, synthetic construct
  source 1..24: mol_type = other DNA
accaggcaagtctcctaagangaa

SEQ ID NO: 129 - DNA, 23 bp, synthetic construct
  source 1..23: mol_type = other DNA
ggcaagtctcctaagaggaanga

SEQ ID NO: 130 - DNA, 23 bp, synthetic construct
  source 1..23: mol_type = other DNA
caccaggcaagtctcctaagngg

SEQ ID NO: 131 - protein, 22 aa, synthetic construct
MSRSVALAVLALLSLSGLEAIQ

SEQ ID NO: 132 - protein, 66 aa, synthetic construct
IQNPDPAVYQLRDSKSSDKSVCLFTDFDSQTNVSQSKDSDVYITDKTVLDMRSMDFKSNSAVAWSN

SEQ ID NO: 133 - DNA, 23 bp, synthetic construct
  source 1..23: mol_type = other DNA
gagtagcgcgagcacagctangg

SEQ ID NO: 134 - DNA, 23 bp, synthetic construct
  source 1..23: mol_type = other DNA
ctcgcgctactctctctttcngg

(19) United States
(12) Patent Application Publication
(10) Pub. No.: US 2025/0242046 A1
(43) Pub. Date: Jul. 31, 2025

(54) ALLOGENEIC HYPOIMMUNE BIOMIMETIC NANOVESICLE FOR THE TREATMENT OF CANCER

(71) Applicant: Thomas MALCOLM, Andover, NJ (US)

(72) Inventor: Thomas MALCOLM, Andover, NJ (US)

(21) Appl. No.: 18/868,324

(22) PCT Filed: May 25, 2023
(86) PCT No.: PCT/US2023/023534

§ 371 Date: Nov. 22, 2024

Related U.S. Application Data
(60) Provisional application No. 63/345,664, filed on May 25, 2022.

(51) Int. Cl. A61K 47/69; A61K 9/19; A61K 40/31; A61K 40/42; A61K 45/06; A61K 47/68; B82Y 5/00; C07K 16/28; C07K 16/30

(52) U.S. Cl. CPC A61K 47/6901; A61K 9/19; A61K 40/31; A61K 40/4202; A61K 45/06; A61K 47/6849; C07K 16/2818; C07K 16/2827; C07K 16/303; B82Y 5/00; C07K 2317/622

(57) ABSTRACT

Disclosed herein are compositions comprising allogeneic, hypoimmunogenic chimeric antigen receptor (CAR)-targetable biomimetic nanovesicles (BioNVs) and methods of using the same for the treatment, prevention, and/or amelioration of cancer.

### CROSS-REFERENCE TO RELATED APPLICATIONS

This application claims the benefit of and priority to U.S. Provisional Application No. 63/345,664, filed May 25, 2022, the contents of which are herein incorporated by reference in its entirety.

### TECHNICAL FIELD

The present disclosure provides, in part, compositions and methods comprising allogeneic, hypoimmunogenic biomimetic nanovesicles and methods of using the same for the treatment or prevention of a cancer, e.g., in a mammalian subject, such as a human.

### BACKGROUND

Chimeric antigen receptor (CAR) whole cell therapies (T cells, NK cells, macrophages, Tumor Infiltrating Lymphocytes (TILs), etc.) have been used to target cells for the treatment of cancer. CAR whole cell therapy is a cancer therapy that generally requires the collection of a patient's own immune cells (T cells) to treat their cancer or the development of allogeneic cell lines from stem cells to treat broader patient populations at lower cost. Lymphocytes normally attack invasive microorganisms, but in CAR whole-cell therapy, the cells are engineered to target cancer cells.

First, the cells are separated from the patient's blood and genetically engineered to express CARs on their surface that allow the cells to bind a specific antigen. Alternatively, the cells are genetically engineered from stem cells (e.g., iPSCs, MSCs, or embryonic stem cells) to incorporate properties of allogenicity and stability from immune system (e.g., incorporation of molecular CD47 transmembrane tag to prevent clearance by macrophages), followed by the stable integration of the desired CAR. The CARs are non-naturally occurring fusion proteins containing fragments of synthetic antibodies. CARs can recognize target cancer biomarkers through several binding moieties: 1) an antibody fragment scFV region, or bifunctional or BiTE antibody format; 2) by a viral epitope recognition receptor (VERR) derived from oncolytic viral receptors; 3) by a camelid-derived variable heavy chain IgG fragment called a VHH single-domain nanobody (VHH nanobody); 4) by a cartilaginous fish-derived variable heavy chain IgG fragment called a Variable New Antigen Receptor (VNAR); 5) by an engineered T cell receptor (TCR); 6) by any single heavy chain IgG fragment from which a variable region can be engineered into a CAR structure to recognize any biomarker; 7) by affilins (artificial binding domain with antigen selectivity); or 8) by chimeric endocrine receptors (CERs). CAR constructs in whole cell therapies rely on engineered signaling and co-stimulatory domains inside the cell to function.

Once the CAR whole cells have been produced, they are expanded to produce large quantities that can then be infused back into the patient (autologous), or if from an engineered stem cell source, infused into a broad spectrum of patients (allogeneic). The CAR whole cells recognize specific tumor antigens on the cancer cells they are designed to kill the cancer cells that have those specific antigens.

CAR T-cell therapies have been approved for the treatment of acute lymphoblastic leukemia (ALL) in children and advanced lymphomas in adults. For example, CAR T cells that target CD-19 (tisagenlecleucel, KYMRIAH®, Novartis) have been approved to treat ALL. YESCARTA® (axicabtagene ciloleucel, Gilead/Kite Pharmaceuticals) is approved for the treatment of lymphomas. Studies have also been conducted to target CD-22 in cells that have lost CD-19 expression. Dual targeting of CD-19 and CD-123 in leukemia has also been studied. CAR T cells that target B cell maturation antigen (BCMA) have recently been approved as the treatment of multiple myeloma (MM) (idecabtagene vicleucel, ADECMA®, Bristol Myers Squibb). It is unclear currently whether CAR T cells can treat solid tumors due to the microenvironment that surrounds them.

Despite the recent advances, there are several drawbacks to CAR whole cell therapy. 1) It can cause cytokine release syndrome (CRS) that results in a systemic inflammatory response from immunotherapy, resulting in high fevers and low blood pressure. This can require additional treatments, for example, blocking IL-6 activity. It can also cause B cell death (B cell aplasia), requiring further treatment with immunoglobulins. Other side effects can include cerebral edema and neurotoxicity. 2) Autologous sourcing requires that the patients to be treated have an adequate number of T cells (or NK or macrophage) to harvest and engineer, which is sometimes not feasible. 3) Multiple rounds of treatment are often required and laborious reengineering can also be required, especially when tumor cells lose antigen expression. 4) Whole cell stability in the tumor microenvironment is poor and presents a major challenge, as the hypoxic and acidic environment can trigger apoptosis. 5) Once a cell enters the tumor environment, it may become ‘hyperactive’ with increased metabolism that leads to exhaustion. 6) T-cell responses can be shut down, for example, through the PD-1 pathway via the upregulation of PD-L1 on tumor cells.

The PD-1 signaling axis blocks key mechanisms of killing normal, healthy cells, and is especially important in the context of whole-cell therapy. There are potential side effects caused by off-targeting in whole cell therapies that occurs when KEYTRUDA® (pembrolizumab, Merck) or OPDIVO® (nivolumab, Bristol-Myers Squibb) block PD-1 on T cells, therefore exposing PD-L1-containing healthy cells to immune clearance, even though PD-L1 is upregulated on tumor cells. Medications that block PD-1 on T cells prevent its interaction with PD-L1 and PD-L2 on cancer cells (upregulated) or healthy cells, and allow the immune system to clear PD-L1 expressing cancer cells. However, off-target effects aimed at healthy cells can occur. KEYTRUDA® (pembrolizumab, Merck) also has other undesirable side effects, such as blurred vision, body aches and pains, confusion, constipation, abnormal stool or urine, depression, headache, nausea, among others.

PD-1 (programmed cell death protein 1) is a protein found on the surface of T cells that helps regulate the immune system's response to cells in the body by downregulating the immune system and promoting self-tolerance by suppressing T cell inflammatory activity. PD-1 on T cells binds PD-L1 and PD-L2 on normal, healthy cells. The PD-1-PD-L1 interaction deactivates the cell-mediated response against normal cells to prevent T cell-mediated killing of healthy cells. This action also prevents the immune system from targeting and killing cancer cells because cancer cells can express upregulated PD-L1. Because T-cell PD-1 binds cancer cells with PD-L1, this potentiates a no-kill signal to the T cell, which hides the cancer cell from the immune system.

Although the PD-1-blocking strategy has had some efficacy in various cancers, the standalone treatment for most solid tumor cancers is relatively ineffective. Several combination/co-therapeutic strategies have been developed to improve clinical outcomes regarding targeting this signaling pathway. Some of these strategies include the coupling/co-therapeutic approaches with anti-PD-1 monoclonal antibodies, potent small molecules, monoclonal antibodies, gene editing, combined with a multitude of delivery systems including AAVs and lipid nanoparticles (LNPs). Most recently, LNPs have been utilized to target PD-L1 on cancer cells while delivering an inhibitory/toxic nucleic, or to deliver an anti-PD-L1 nucleic acid (such as mRNA). Moderna, for example, has a checkpoint cancer vaccine (mRNA-4359), which expresses indoleamine 2,3-dioxygenase and PD-L1 antigens in order to stimulate effector T cells that target and kill immune and tumor cells expressing the target antigens. This latter approach may achieve improved efficacies, but the accessibility of the LNP to the solid tumor mass within the tumor microenvironment (TME) may be limited and delivery of the toxic or anti-PD-L1 nucleic acid may be further limited due to the lack of penetration into tumor cells.

There remains a need for cancer treatments that overcome the problems of addressing PD-L1/PD-1 signaling issues and overcomes the drawbacks of standard whole cell therapies in treating cancer, while taking advantage of the targeting and killing efficacy of whole cell therapies.

### SUMMARY

In embodiments, a method of treating or preventing a cancer comprising administering to a subject in need thereof (i) a therapeutically effective amount of a biomimetic nanovesicle (BioNV) comprising (a) a membrane-embedded chimeric antigen receptor (CAR) targeted against a cell surface marker, and (b) a PD-1, PD-L1, and/or PD-L2 inhibiting agent; or (ii) a therapeutically effective amount of a BioNV comprising a membrane-embedded CAR targeted against a cell surface marker, wherein the subject is undergoing treatment with a PD-1, PD-L1, and/or PD-L2 inhibiting agent.

In embodiments, a method of treating or preventing a cancer comprising administering to a subject in need thereof (i) a therapeutically effective amount of a biomimetic nanovesicle (BioNV) comprising (a) a membrane-embedded chimeric antigen receptor (CAR) targeted against a cell surface marker, and a conjugated and/or membrane-anchored PD-L1 and/or PD-L2 inhibiting agent; or (ii) a therapeutically effective amount of a BioNV comprising (a) a membrane-embedded bispecific CAR targeted against a first cell surface marker and a second cell surface marker, and a conjugated and/or membrane-anchored PD-L1 and/or PD-L2 inhibiting agent.

In embodiments, a method of treating or preventing a cancer comprising administering to a subject in need thereof (i) a therapeutically effective amount of a biomimetic nanovesicle (BioNV) comprising a bispecific chimeric antigen receptor (CAR) targeted against a first cell surface marker and either PD-L1 or PD-L2, and wherein the first cell surface marker is not PD-1, PD-L1, or PD-L2; or (ii) a therapeutically effective amount of a BioNV comprising a bispecific CAR targeted against a first cell surface marker and either PD-L1 or PD-L2, and wherein the first cell surface marker is not PD-1, PD-L1 or PD-L2, and a PD-1, PD-L1, and/or PD-L2 inhibiting agent; or (iii) a therapeutically effective amount of a BioNV comprising a bispecific CAR targeted against either PD-L1 or PD-L2 and a first cell surface marker, wherein the subject is undergoing treatment with a PD-1, PD-L1, and/or PD-L2 inhibiting agent, and wherein the first cell surface marker is not PD-1, PD-L1, or PD-L2.

In embodiments, a method of treating or preventing a cancer comprising administering to a subject in need thereof a therapeutically effective amount of a biomimetic nanovesicle (BioNV) comprising a bispecific chimeric antigen receptor (CAR) targeted against a first cell surface marker and a second cell surface marker, and a PD-1, PD-L1, and/or PD-L2 inhibiting agent.

In embodiments, a method of treating or preventing a cancer comprising administering to a subject in need thereof a therapeutically effective amount of a biomimetic nanovesicle (BioNV) comprising a bispecific chimeric antigen receptor (CAR) targeted against a first cell surface marker and a second cell surface marker, wherein the subject is undergoing treatment with a PD-1, PD-L1, and/or PD-L2 inhibiting agent.

In embodiments, the first cell surface marker and the second cell marker are selected from Table 1 and/or Table 2. In embodiments, the first cell surface marker selected from Table 1 and/or Table 2. In embodiments, the BioNV binds at least a first cell and at least a second cell to initiate an anti-cancer response. In embodiments, the first cell is a cancer cell, and the second cell is an immune cell. In embodiments, the PD-1 inhibiting agent is an antibody targeted against PD-1, optionally pembrolizumab, nivolumab, or cemiplimab. In embodiments, the PD-L1 and/or PD-L2 inhibiting agent is an antibody targeted against PD-L1 and/or PD-L2, optionally atezolizumab, avelumab, or durvalumab.

In embodiments, the BioNV originates from a modified cell. In embodiments, the modified cell is a stem cell, an induced pluripotent stem cell (iPSC), a reprogrammed pluripotent or multipotent cell, an embryonic stem cell, a mesenchymal stem cell, or a differentiated cell which originates from any modified cell thereof. In embodiments, the modified cell is an iPSC. In embodiments, the modified cell is a T cell, helper T cell, T-memory cell, or NK cell. In embodiments, the modified cell is a macrophage. In embodiments, the modified cell is a monocyte. In embodiments, the hypoimmunogenic cell can be any terminally differentiated cell, for example and without limitation, a muscle cell (satellite cell), adipocyte, osteocyte, cardiomyocyte, hepatocyte, blood cell (including erythrocyte, thrombocyte, and all immune cell types), glial cell (among other neuronal cell types), epithelial cell, epidermal cell, interstitial cell (e.g., respiratory interstitial cell), fibroblast (e.g., dermal fibroblast), endothelial cell (e.g., bronchial endothelial cell), oral cell, stromal cell, or germ cell. In embodiments, the hypoimmunogenic cell can be any function-specific cell type, for example and without limitation, exocrine secretory epithelial cell, hormone-secreting cell (e.g. enteroendocrine cell, thyroid cell, pancreatic islet cell, etc.), sensory transducer cell, autonomic neuronal cell, sensory organ cell (e.g., pillar cell, olfactory cell, Schwann cell, satellite glial cell, etc.), barrier cell (e.g., pneumocyte, duct cell, kidney cell, podocyte, etc.), extracellular matrix cell (e.g., tendon fibroblast, osteoblast, connective tissue cell, etc.), or contractile cell (e.g., skeletal muscle cell, cardiac muscle cell, myoepithelial cell, etc.).

In embodiments, the modified cell substantially lacks one or more MHC class I proteins, MHC class II proteins, T cell receptor (TCR) proteins, and/or cytokine release syndrome (CRS) proteins. In embodiments, the modified cell has reduced or ablated expression of a 32-macroglobulin (B2M) gene and/or reduced or ablated MHC class I protein expression and/or activity. In embodiments, the modified cell has reduced or ablated expression of a CIITA gene and/or reduced or ablated MHC class II protein expression and/or activity.

In embodiments, the modified cell has reduced or ablated expression of an HLA-A gene and/or reduced or ablated HLA-A protein expression and/or activity. In embodiments, the modified cell has reduced or ablated expression of an HLA-B gene and/or reduced or ablated HLA-B protein expression and/or activity. In embodiments, the modified cell has reduced or ablated expression of an HLA-C gene and/or reduced or ablated HLA-C protein expression and/or activity. In embodiments, the modified cell has reduced or ablated expression of an HLA-E or HLA-G gene and/or reduced or ablated HLA-E or HLA-G protein expression and/or activity. In embodiments, the modified cell has reduced or ablated expression of an HLA-F gene and/or reduced or ablated HLA-F protein expression and/or activity.

In embodiments, the modified cell has reduced or ablated expression of a T cell alpha constant (TRAC) gene and/or reduced or ablated TRAC protein expression and/or activity. In embodiments, the modified cell has reduced or ablated expression of a T cell beta constant (TRBC) gene and/or reduced or ablated TRBC protein expression and/or activity.

In embodiments, the modified cell has reduced or ablated expression of a PD-1 gene and/or reduced or ablated PD-1 protein expression and/or activity.

In embodiments, the modified cell has reduced or ablated expression of an IL-4 gene and/or reduced or ablated IL-4 protein expression and/or activity. In embodiments, the modified cell has reduced or ablated expression of an IL-6 gene and/or reduced or ablated IL-6 protein expression and/or activity. In embodiments, the modified cell has reduced or ablated expression of an IL-10 gene and/or reduced or ablated IL-10 protein expression and/or activity. In embodiments, the modified cell has reduced or ablated expression of an IL-16 gene and/or reduced or ablated IL-16 protein expression and/or activity.

In embodiments, the modified cell has reduced or ablated expression of a SerpinB9 gene and/or reduced or ablated SerpinB9 protein expression and/or activity.

In embodiments, the modified cell expresses or has increased expression of a CD34 gene and/or gene product. In embodiments, the modified cell expresses or has increased expression of a CCL2 gene and/or gene product. In embodiments, the modified cell expresses or has increased expression of a PD-L1 gene and/or gene product, and wherein the modified cell is not activated. In embodiments, the modified cell has reduced or ablated expression of a PD-L1 gene and/or gene product, and wherein the modified cell is activated. In embodiments, the modified cell overexpresses FasL. In embodiments, the modified cell overexpresses SerpinB9. In embodiments, the modified cell expresses or has increased expression of a H2-M3 gene and/or gene product.

In embodiments, the modified cell expresses or has increased expression of a CD47 gene and/or gene product. In embodiments, the modified cell expresses or has increased expression of a CD24 gene and/or gene product. In embodiments, the modified cell expresses or has increased expression of a chimeric CD24/CD47 gene and/or gene product.

In embodiments, the modified cell overexpresses CTLA-4. In embodiments, the modified cell expresses or has increased expression of a chimeric CD200 gene and/or gene product. In embodiments, the modified cell expresses or has increased expression of a chimeric CD24/CD200 gene and/or gene product or a chimeric CD47/CD200 gene and/or gene product.

In embodiments, the modified cell expresses or has increased expression of a chimeric MFG-E8 gene and/or gene product. In embodiments, the modified cell expresses or has increased expression of an NCAM gene and/or gene product. In embodiments, the modified cell expresses or has increased expression of a chimeric α-phagocytic integrin gene and/or gene product. In embodiments, the modified cell expresses or has increased expression of an antibody or antibody format molecule which targets an IL-6 surface receptor (anti-IL-6R).

In embodiments, the modified cell expresses or has increased expression of a FasL gene and/or gene product. In embodiments, the modified cell wherein does not overexpress a FasL gene and/or gene product.

In embodiments, the modified cell has reduced or ablated expression and/or activity of 3 or more immunogenic proteins, 4 or more immunogenic proteins, 5 or more immunogenic proteins, 6 or more immunogenic proteins, 7 or more immunogenic proteins, 8 or more immunogenic proteins, 9 or more immunogenic proteins, 10 or more immunogenic proteins, 11 or more immunogenic proteins, or 12 or more immunogenic proteins. In embodiments, the modified cell expresses or has increased expression of 3 or more immunoprotective proteins, 4 or more immunoprotective proteins, 5 or more immunoprotective proteins, 6 or more immunoprotective proteins, 7 or more immunoprotective proteins, 8 or more immunoprotective proteins, 9 or more immunoprotective proteins, or 10 or more immunoprotective proteins.

In embodiments, the modified cell is allogeneic. In embodiments, the modified cell does not cause an immune reaction in patients to which it or a BioNV derived therefrom is administered.

In embodiments, the modified cell is differentiated prior to BioNV formation.

In embodiments, the modified cell expresses the CAR under the control of a regulatable expression element. In embodiments, the CAR is activated prior to BioNV formation. In embodiments, the CAR is activated via its target, through another receptor, and/or a virus.

In embodiments, the BioNV is formed from a modified cell, or a differentiated cell thereof, by sonication, adaptive focused acoustics technology, French press, extrusion, serial extrusion, cell lysis by detergent, and/or electroporation. In embodiments, the BioNV is formed from the modified cell, or the differentiated cell thereof, by serial extrusion.

In embodiments, the BioNV is about 10 nm to about 1200 nm in size. In embodiments, the BioNV is about 10 nm to about 100 nm in size. In embodiments, the BioNV is about 100 nm to about 200 nm in size. In embodiments, the BioNV is about 200 nm to about 500 nm in size. In embodiments, wherein the BioNV is about 500 nm to about 1200 nm in size.

In embodiments, the BioNV substantially lacks protein and/or activity of HLA-A, HLA-B, HLA-C, HLA-F, CIITA, IL-6, TRAC, TRBC, and one of either HLA-E or HLA-G.

In embodiments, the BioNV substantially lacks protein and/or activity of HLA-A, HLA-B, HLA-C, HLA-F, CIITA, IL-6, TRAC, TRBC, SerpinB9, and one of either HLA-E or HLA-G.

In embodiments, the BioNV substantially lacks protein and/or activity of HLA-A, HLA-B, HLA-C, HLA-F, CIITA, IL-6, TRAC, TRBC, SerpinB9, CD200, one of either HLA-E or HLA-G, and one or more of IL-4, IL-10, and IL-16.

In embodiments, the BioNV has a membrane-embedded α-phagocytic integrin, CCL2, H2-M3, FasL, MFG-E8, anti-IL-6R antibody or antibody format, and PD-L1 (in BioNVs derived from non-activated cell sources) and/or CTLA-4, and one of either CD24, CD47, CD200, chimeric CD24/CD47, chimeric CD24/CD200, and chimeric CD47/CD200, or two of either CD24, CD47, and CD200.

In embodiments, the BioNV has a membrane-embedded α-phagocytic integrin, CCL2, H2-M3, FasL, MFG-E8, anti-IL-6R antibody or antibody format, SerpinB9, and PD-L1 (in BioNVs derived from non-activated cell sources) and/or CTLA-4, and one of either CD24, CD47, CD200, chimeric CD24/CD47, chimeric CD24/CD200, and chimeric CD47/CD200, or two of either CD24, CD47, and CD200.

In embodiments, the BioNV has a membrane-embedded CD200 protein and substantially lacks protein of either CD24 or CD47. In embodiments, the BioNV substantially lacks protein and/or activity of SerpinB9 and CD200.

In embodiments, the CAR comprises an antibody or antibody format selected from one or more of a monoclonal antibody, polyclonal antibody, antibody fragment, V_{NAR}, V_{H}H, Fab, Fab′, Fab′-SH, F(ab′)2, Fv, single chain Fv (scFv), diabody, nanobody, linear antibody, bispecific antibody, multi-specific antibody, chimeric antibody, humanized antibody, human antibody, or fusion protein comprising the antigen-binding portion of an antibody. In embodiments, the antibody format is the scFv. In embodiments, the CAR comprises a transmembrane domain derived from CD28, CD3ζ, CD4, CD8α, ICOS, or fragment and/or combination thereof. In embodiments, the CAR comprises an intracellular domain further comprising an intracellular signaling domain of a CD3ζ-chain and/or one or more co-stimulatory molecules, optionally selected from CD28, 4-1BB, ICOS, CD27, and OX40. In embodiments, the CAR is targeted against a cancer-specific antigen.

In embodiments, the BioNV, or the modified cell derived therefrom, comprises a nucleic acid encoding green fluorescence protein (GFP) and/or a GFP protein. In embodiments, the nucleic acid encoding GFP is operably linked to a promoter from one or more of IL-2, perforin, granzyme, alarmin, TNF, INF, and/or a combination thereof.

In embodiments, the BioNV encapsulates a payload. In embodiments, the payload is one or more of a gene editor, cytotoxic protein, biologic, nucleic acid, fusion protein, fluorescent protein, tracing dye, radionuclide, and/or small molecule. In embodiments, the payload is a therapeutic payload for a cancer type that the CAR is targeted against. In embodiments, the payload comprises an alkylating agent. In embodiments, the payload comprises an anthracycline. In embodiments, the payload comprises an antimetabolite. In embodiments, the payload comprises an anti-tumor antibiotic. In embodiments, the payload comprises an anti-tumor antibody or antibody format. In embodiments, the payload comprises a corticosteroid. In embodiments, the payload comprises a plant alkaloid. In embodiments, the payload comprises a topoisomerase inhibitor. In embodiments, the payload comprises a checkpoint inhibitor.

In embodiments, the payload is selected from Abecma, Abemaciclib, Abiraterone Acetate, Abraxane, ABVD, ABVE, ABVE-PC, AC, Acalabrutinib, AC-T, Actemra, Adcetris, ADE, Ado-Trastuzumab Emtansine, Adriamycin, Afatinib Dimaleate, Afinitor, Akynzeo, Aldara, Aldesleukin, Alecensa, Alectinib, Alemtuzumab, Alimta, Aliqopa, Alkeran for Injection, Alkeran Tablets, Aloxi, Alpelisib, Alunbrig, Ameluz, Amifostine, Aminolevulinic Acid Hydrochloride, Amivantamab-vmjw, Anastrozole, Apalutamide, Aprepitant, Aranesp, Aredia, Arimidex, Aromasin, Arranon, Arsenic Trioxide, Arzerra, Asciminib Hydrochloride, Asparaginase Erwinia Chrysanthemi, Asparlas, Atezolizumab, Avapritinib, Avastin, Avelumab, Axicabtagene Ciloleucel, Axitinib, Ayvakit, Azacitidine, Azedra, Balversa, Bavencio, BEACOPP, Belantamab Mafodotin-blmf, Beleodaq, Belinostat, Belzutifan, Bendamustine Hydrochloride, Bendeka, BEP, Besponsa, Besremi, Bevacizumab, Bexarotene, Bicalutamide, BiCNU, Binimetinib, Blenrep, Bleomycin Sulfate, Blinatumomab, Blincyto, Bortezomib, Bosulif, Bosutinib, Braftovi, Brentuximab Vedotin, Brexucabtagene Autoleucel, Breyanzi, Brigatinib, Brukinsa, BuMel, Busulfan, Busulfex, Cabazitaxel, Cablivi, Cabometyx, Cabozantinib-S-Malate, CAF, Calaspargase Pegol-mknl, Calquence, Campath, Camptosar, Capecitabine, Caplacizumab-yhdp, Capmatinib Hydrochloride, CAPOX, Carac, Carboplatin, CARBOPLATIN-TAXOL, Carfilzomib, Carmustine, Carmustine Implant, Casodex, CEM, Cemiplimab-rwlc, Ceritinib, Cerubidine, Cervarix (Recombinant HPV Bivalent Vaccine), Cetuximab, CEV, Chlorambucil, CHLORAMBUCIL-PREDNISONE, CHOP, Cisplatin, Cladribine, Clofarabine, Clolar, CMF, Cobimetinib Fumarate, Cometriq, Copanlisib Hydrochloride, COPDAC, Copiktra, COPP, COPP-ABV, Cosmegen, Cotellic, Crizotinib, CVP, Cyclophosphamide, Cyramza, Cytarabine, Dabrafenib Mesylate, Dacarbazine, Dacogen, Dacomitinib, Dactinomycin, Danyelza, Daratumumab, Daratumumab and Hyaluronidase-fihj, Darbepoetin Alfa, Darolutamide, Darzalex, Darzalex Faspro, Dasatinib, Daunorubicin Hydrochloride, Daunorubicin Hydrochloride and Cytarabine Liposome, Daurismo, Decitabine, Decitabine and Cedazuridine, Defibrotide Sodium, Defitelio, Degarelix, Denileukin Diftitox, Denosumab, Dexamethasone, Dexrazoxane Hydrochloride, Dinutuximab, Docetaxel, Dostarlimab-gxly, Doxil, Doxorubicin Hydrochloride, Doxorubicin Hydrochloride Liposome, Durvalumab, Duvelisib, Efudex, Eligard, Elitek, Ellence, Elotuzumab, Eloxatin, Eltrombopag Olamine, Elzonris, Emapalumab-Izsg, Emend, Empliciti, Enasidenib Mesylate, Encorafenib, Enfortumab Vedotin-ejfv, Enhertu, Entrectinib, Enzalutamide, Epirubicin Hydrochloride, EPOCH, Epoetin Alfa, Epogen, Erbitux, Erdafitinib, Eribulin Mesylate, Erivedge, Erleada, Erlotinib Hydrochloride, Erwinaze, Ethyol, Etopophos, Etoposide, Etoposide Phosphate, Everolimus, Evista, Evomela, Exemestane, Exkivity, 5-FU, 5-FU, Fam-Trastuzumab Deruxtecan-nxki, Fareston, Faslodex, FEC, Fedratinib Hydrochloride, Femara, Filgrastim, Firmagon, Fludarabine Phosphate, Fluoroplex, Fluorouracil Injection, Fluorouracil-Topical, Flutamide, FOLFIRI, FOLFIRI-BEVACIZUMAB, FOLFIRI-CETUXIMAB, FOLFIRINOX, FOLFOX, Folotyn, Fostamatinib Disodium, Fotivda, Fulphila, FU-LV, Fulvestrant, Fyarro, Gamifant, Gardasil (Recombinant HPV Quadrivalent Vaccine), Gardasil 9 (Recombinant HPV Nonavalent Vaccine), Gavreto, Gazyva, Gefitinib, Gemcitabine Hydrochloride, GEMCITABINE-CISPLATIN, GEMCITABINE-OXALIPLATIN, Gemtuzumab Ozogamicin, Gemzar, Gilotrif, Gilteritinib Fumarate, Glasdegib Maleate, Gleevec, Gliadel Wafer, Glucarpidase, Goserelin Acetate, Granisetron, Granisetron Hydrochloride, Granix, Halaven, Hemangeol, Herceptin Hylecta, Herceptin, HPV Bivalent Vaccine, Recombinant, HPV Nonavalent Vaccine, Recombinant, HPV Quadrivalent Vaccine, Recombinant, Hycamtin, Hydrea, Hydroxyurea, Hyper-CVAD, Ibrance, Ibritumomab Tiuxetan, Ibrutinib, ICE, Iclusig, Idamycin PFS, Idarubicin Hydrochloride, Idecabtagene Vicleucel, Idelalisib, Idhifa, Ifex, Ifosfamide, Interleukin-2 (recombinant), Imatinib Mesylate, Imbruvica, Imfinzi, Imiquimod, Imlygic, Infigratinib Phosphate, Infugem, Inlyta, Inotuzumab Ozogamicin, Inqovi, Inrebic, Interferon Alfa-2b, Intron A, Iobenguane I131, Ipilimumab, Iressa, Irinotecan Hydrochloride, Isatuximab-irfc, Istodax, Ivosidenib, Ixabepilone, Ixazomib Citrate, Ixempra, Jakafi, JEB, Jelmyto, Jemperli, Jevtana, Kadcyla, Kepivance, Keytruda, Kimmtrak, Kisqali, Koselugo, Kymriah, Kyprolis, Lanreotide Acetate, Lapatinib Ditosylate, Larotrectinib Sulfate, Lenalidomide, Lenvatinib Mesylate, Lenvima, Letrozole, Leucovorin Calcium, Leukeran, Leuprolide Acetate, Levulan Kerastik, Libtayo, Lisocabtagene Maraleucel, Lomustine, Loncastuximab Tesirine-lpyl, Lonsurf, Lorbrena, Lorlatinib, Lumakras, Lumoxiti, Lupron Depot, Lurbinectedin, Luspatercept-aamt, Lutathera, Lutetium (Lu 177-Dotatate), Lynparza, Margenza, Margetuximab-cmkb, Marqibo, Matulane, Mechlorethamine Hydrochloride, Megestrol Acetate, Mekinist, Mektovi, Melphalan, Melphalan Hydrochloride, Mercaptopurine, Mesna, Mesnex, Methotrexate Sodium, Methylnaltrexone Bromide, Midostaurin, Mitomycin, Mitoxantrone Hydrochloride, Mobocertinib Succinate, Mogamulizumab-kpkc, Monjuvi, MOPP, Moxetumomab Pasudotox-tdfk, Mozobil, MVAC, Mvasi, Myleran, Mylotarg, Nanoparticle Paclitaxel, Naxitamab-gqgk, Necitumumab, Nelarabine, Neratinib Maleate, Nerlynx, Netupitant, Neulasta, Neupogen, Nexavar, Nilandron, Nilotinib, Nilutamide, Ninlaro, Niraparib Tosylate Monohydrate, Nivestym, Nivolumab, Nplate, Nubeqa, Nyvepria, Obinutuzumab, Odomzo, OEPA, Ofatumumab, OFF, Olaparib, Omacetaxine Mepesuccinate, Oncaspar, Ondansetron Hydrochloride, Onivyde, Ontak, Onureg, Opdivo, OPPA, Orgovyx, Osimertinib Mesylate, Oxaliplatin, Paclitaxel, Paclitaxel Albumin-stabilized Nanoparticle Formulation, PAD, Padcev, Palbociclib, Palifermin, Palonosetron Hydrochloride, Pamidronate Disodium, Panitumumab, Paraplatin, Pazopanib Hydrochloride, PCV, PEB, Pegaspargase, Pegfilgrastim, Peginterferon Alfa-2b, PEG-Intron, Pemazyre, Pembrolizumab, Pemetrexed Disodium, Pemigatinib, Perjeta, Pertuzumab, Pexidartinib Hydrochloride, Phesgo, Piqray, Plerixafor, Polatuzumab Vedotin-piiq, Polivy, Pomalidomide, Pomalyst, Ponatinib Hydrochloride, Portrazza, Poteligeo, Pralatrexate, Pralsetinib, Prednisone, Procarbazine Hydrochloride, Procrit, Proleukin, Prolia, Promacta, Propranolol Hydrochloride, Provenge, Purinethol, Purixan, Qinlock, Radium 223 Dichloride, Raloxifene Hydrochloride, Ramucirumab, Rasburicase, Ravulizumab-cwvz, Reblozyl, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor, Relugolix, R-EPOCH, Retacrit, Retevmo, Revlimid, Ribociclib, R-ICE, Ripretinib, Rituxan, Rituxan Hycela, Rituximab, Rolapitant Hydrochloride, Romidepsin, Romiplostim, Ropeginterferon Alfa-2b-njft, Rozlytrek, Rubidomycin, Rubraca, Rucaparib Camsylate, Ruxolitinib Phosphate, Rybrevant, Rydapt, Rylaze, Sacituzumab Govitecan-hziy, Sancuso, Sarclisa, Sclerosol Intrapleural Aerosol, Selinexor, Selpercatinib, Selumetinib Sulfate, Scemblix, Siltuximab, Sipuleucel-T, Sirolimus Protein-Bound Particles, Soltamox, Somatuline Depot, Sonidegib, Sorafenib Tosylate, Sotorasib, Sprycel, STANFORD V, Sterile Talc Powder, Steritalc, Stivarga, Sunitinib Malate, Sustol, Sutent, Sylatron, Sylvant, Synribo, Tabloid, Tabrecta, TAC, Tafasitamab-cxix, Tafinlar, Tagraxofusp-erzs, Tagrisso, Talazoparib Tosylate, Talimogene Laherparepvec, Talzenna, Tamoxifen Citrate, Tarceva, Targretin, Tasigna, Tavalisse, Taxotere, Tazemetostat Hydrobromide, Tazverik, Tebentafusp-tebn, Tecartus, Tecentriq, Temodar, Temozolomide, Temsirolimus, Tepadina, Tepmetko, Tepotinib Hydrochloride, Thalidomide, Thalomid, Thioguanine, Thiotepa, Tibsovo, Tisagenlecleucel, Tisotumab Vedotin-tftv, Tivdak, Tivozanib Hydrochloride, Tocilizumab, Tolak, Topotecan Hydrochloride, Toremifene, Torisel, Totect, TPF, Trabectedin, Trametinib Dimethyl Sulfoxide, Trastuzumab, Trastuzumab and Hyaluronidase-oysk, Treanda, Trexall, Trifluridine and Tipiracil Hydrochloride, Trisenox, Trodelvy, Truseltiq, Truxima, Tucatinib, Tukysa, Turalio, Tykerb, Ukoniq, Ultomiris, Umbralisib Tosylate, Undencyca, Unituxin, Uridine Triacetate, VAC, Valrubicin, Valstar, Vandetanib, VAMP, Varubi, Vectibix, VelP, Velcade, Vemurafenib, Venclexta, Venetoclax, Verzenio, Vidaza, Vinblastine Sulfate, Vincristine Sulfate, Vincristine Sulfate Liposome, Vinorelbine Tartrate, VIP, Vismodegib, Vistogard, Vitrakvi, Vizimpro, Voraxaze, Vorinostat, Votrient, Vyxeos, Welireg, Xalkori, Xatmep, Xeloda, XELIRI, XELOX, Xgeva, Xofigo, Xospata, Xpovio, Xtandi, Yervoy, Yescarta, Yondelis, Yonsa, Zaltrap, Zanubrutinib, Zarxio, Zejula, Zelboraf, Zepzelca, Zevalin, Ziextenzo, Zinecard, Zirabev (Bevcizumab), Ziv-Aflibercept, Zofran, Zoladex, Zoledronic Acid, Zolinza, Zometa, Zyclara, Zydelig, Zykadia, Zynlonta, and Zytiga.

In embodiments, the payload is one or more of pidilizumab, BMS-936559, tremelimumab, AGEN1884, and/or RG2077.

In embodiments, the nucleic acid molecule encodes one or more of a CRISPR/Cas component, guide RNA (gRNA), tracer RNA (tracrRNA), micro RNA (miRNA), RNA inference (RNAi), small interference RNA (siRNA), duplex RNA, Piwi-interacting RNA (piRNA), small nuclear RNA (snRNA), small nucleolar RNA (snoRNA), antisense oligonucleotide (ASO), locked nucleic acid (LNA), splice switching oligonucleotide (SSO), tRNA, complementary messenger RNA, repeat associated small interfering RNA (rasiRNA), and/or small non-coding RNA. In embodiments, the gene editing payload is one or more of a TALEN, ZFN, RNase P RNA, C2c1, C2c2, C2c3, Cas9, Cpf1, TevCas9, Archaea Cas9, CasY.1, CasY.2, CasY.3, CasY.4, CasY.5, CasY.6, CasX Cas omega, transposase, and/or any ortholog or homolog thereof.

In embodiments, the BioNV encapsulates one or more perforin molecules. In embodiments, the BioNV encapsulates one or more granzyme molecules. In embodiments, the granzyme molecules are selected from granzyme A, B, H, K, and M. In embodiments, the BioNV encapsulates one or more perforin molecules and/or one or more granzyme molecules derived from a cell from which the BioNV is derived. In embodiments, the BioNV encapsulates one or more perforin molecules and one and/or more granzyme molecules exogenously added to the BioNV.

In embodiments, methods of treatment further comprise co-administering a whole cell therapy. In embodiments, methods of treatment further comprise administering an additional therapeutic agent.

In embodiments, the BioNV is stored at about −80° C. or is suitable for storage at about −80° C. In embodiments, the BioNV is lyophilized.

In embodiments, the PD-1 inhibiting agent is an antibody targeted against PD-1 or pembrolizumab, nivolumab, or cemiplimab. In embodiments, the PD-L1 or PD-L2 inhibiting agent is an antibody targeted against PD-L1 or PD-L2, optionally atezolizumab, avelumab, or durvalumab, or the antigen-binding domain thereof, and is conjugated to the surface of the BioNV. In embodiments, the PD-1 inhibiting agent is an antibody targeted against PD-1, or pembrolizumab, nivolumab, or cemiplimab. In embodiments, the PD-L1 or PD-L2 inhibiting agent is an antibody targeted against PD-L1 or PD-L2, optionally atezolizumab, avelumab, or durvalumab, or the antigen-binding domain thereof, and is membrane-anchored via a transmembrane domain fusion.

In embodiments, the cancer is a carcinoma. In embodiments, the cancer is a sarcoma. In embodiments, the cancer is a myeloma. In embodiments, the cancer is a leukemia. In embodiments, the cancer is a lymphoma. In embodiments, the cancer is a mixed type cancer. In embodiments, the cancer is metastatic.

In embodiments, the cancer is selected from Acute Biphenotypic Leukemia, Acute Eosinophilic Leukemia, Acute Lymphoblastic Leukemia, Acute Lymphocytic Leukemia, Acute Myeloid Dendritic Cell Leukemia, Acute Myeloid Leukemia, Adenocarcinoma of the Lung, Adrenal Gland Tumors, Adrenocortical Carcinoma, AIDS-related Cancers, AIDS-related Lymphoma, Alveolar Soft Part and Cardiac Sarcoma, Amyloidosis, Anal Cancer, Anaplastic Large Cell Lymphoma, Angioimmunoblastic T-cell Lymphoma, Appendix Cancer, Astrocytoma, Ataxia-telangiectasia, Attenuated Familial Adenomatous Polyposis, B-cell Prolymphocytic Leukemia, Basal Cell Carcinoma, Beckwith-Wiedemann Syndrome, Bile Duct Cancer, Birt-Hogg-Dubé Syndrome, Bladder Cancer, Bone Cancer, Brain and Nervous System Cancer, Brain Stem Glioma, Brainstem Glioma, Brain Tumors, Breast Cancer, Bronchial Adenomas/Carcinoids, Burkitt's Lymphoma, Carcinoid Tumor, Carcinoid Tumors, Carney Complex, Central Nervous System Tumors, Cerebellar Astrocytoma, Cerebral Astrocytoma, Cervical Cancer, Childhood Desmoplastic Ganglioglioma, Cholangiocarcinoma, Chondrosarcoma, Chronic Lymphocytic Leukemia, Chronic Myelogenous Leukemia, Chronic Myeloid Leukemia, Chronic T-cell Lymphocytic Leukemia, Colon Cancer, Colorectal Cancer, Cowden Syndrome, Craniopharyngioma, Cutaneous T-cell Lymphoma, Dermatofibrosarcoma Protuberans, Desmoplastic Small Round Cell Tumor, Diffuse Gastric Cancer, Diffuse Large B-cell Lymphoma, Endocrine System Cancer, Endocrine Tumors, Endometrial Cancer, Eosinophilic Leukemia, Ependymoma, Epithelioid Hemangioendothelioma (EHE), Esophageal Cancer, Ewing Sarcoma, Extragonadal Germ Cell Tumor, Extrahepatic Bile Duct Cancer, Eye Cancer, Eyelid Cancer, Fallopian Tube Cancer, Familial Adenomatous Polyposis, Familial Malignant Melanoma, Familial Clear Cell Renal Cell Carcinoma (RCC), Follicular Lymphoma, Gallbladder Cancer, Gardner Syndrome, Gastric (stomach) Cancer, Gastrointestinal Cancer, Gastrointestinal Carcinoid Tumor, Gastrointestinal Stromal Tumor, Gastrointestinal Stromal Tumor (GIST), Genitourinary and Gynecologic Cancer, Germ Cell Tumor, Gestational Trophoblastic Disease, Gestational Trophoblastic Tumor, Glioblastoma, Glioma, Hairy Cell Leukemia, Head and Neck Cancer, Hematopoietic Cancer, Hepatocellular Cancer, Hepatosplenic T-cell Lymphoma, HIV-related Cancer, Hodgkin's Lymphoma, Hypopharyngeal Cancer, Inflammatory Breast Cancer, Intravascular Large B-cell Lymphoma, Invasive Cribriform Carcinoma, Invasive Lobular Carcinoma, Islet Cell Carcinoma (endocrine Pancreas), Islet Cell Tumors, Juvenile Polyposis Syndrome, Kaposi Sarcoma, Keratoacanthoma, Kidney Cancer, Lacrimal Gland Tumor, Large Granular Lymphocytic Leukemia, Laryngeal and Hypopharyngeal Cancer, Leiomyomatosis and Renal Cell Cancer, Leiomyosarcoma, Li-Fraumeni Syndrome (LFS), Liposarcoma, Liver Cancer, Lung Cancer, Lymphomas of Primary Cutaneous Origin, Lymphomatoid Granulomatosis, Lymphoplasmacytic Lymphoma, Lynch Syndrome, Malignant Fibrous Histiocytoma of Bone, Mantle Cell Lymphoma, Marginal Zone B-cell Lymphoma, Mast Cell Leukemia, Mastocytosis, Mediastinal Large B Cell Lymphoma, Medullary Carcinoma, Medulloblastoma, Melanoma, Meningioma, Merkel Cell Carcinoma, Mesothelioma, Mixed Polyposis Syndrome, Mucosa-associated Lymphoid Tissue Lymphoma, Muir-Torre Syndrome (MTS), Multiple Endocrine Neoplasia Syndrome, Multiple Endocrine Neoplasia Type 1, Multiple Endocrine Neoplasia Type 2, Multiple Myeloma, Multiple Myeloma/Plasma Cell Neoplasm, Mycosis Fungoides, Myelodysplastic Syndromes, MYH-associated Polyposis, Myxosarcoma, Nasal Cavity and Paranasal Sinus Cancer, Nephroblastoma, Nasopharyngeal Cancer, Nasopharyngeal Carcinoma, Neuroblastoma, Neuroendocrine Tumors, Neurofibromatosis Type 1, Neurofibromatosis Type 2, Nevoid Basal Cell Carcinoma Syndrome, Nodal Marginal Zone B Cell Lymphoma, Non-Hodgkin Lymphoma, Non-small Cell Lung Cancer, Non-small Cell Lung Carcinoma, Oligodendroglioma, Optic Nerve Glioma, Oral and/or Oropharyngeal Cancer, Osteosarcoma, Ovarian Cancer, Ovarian Epithelial Cancer, Ovarian Germ Cell Tumor, Pancreatic Cancer, Papillary Renal Cell Carcinoma, Paranasal Sinus and Nasal Cavity Cancer, Parathyroid Cancer, Pelvic Cancer, Penile Cancer, Peutz-Jeghers Syndrome, Pharyngeal Cancer, Pheochromocytoma, Phyllodes Tumor, Pilocytic Astrocytoma, Pineal Astrocytoma, Pituitary Adenoma, Pituitary Gland Tumors, Plasmablastic Lymphoma, Pleuropulmonary Blastoma, Precursor B Lymphoblastic Leukemia, Primary Central Nervous System Lymphoma, Primary Cutaneous Follicular Lymphoma, Primary Cutaneous Immunocytoma, Primary Effusion Lymphoma, Primitive Neuroectodermal Tumor, Prostate Cancer, Rectal Cancer, Renal Cancer, Renal Cell Carcinoma, Retinoblastoma, Rhabdomyosarcoma, Salivary Gland Cancer, Sarcoma, Sarcomas of Primary Cutaneous Origin, Sebaceous Carcinoma, Sezary Syndrome, Skin Adnexal Tumors, Skin Cancer, Small Bowel Cancer, Small Cell Lung Cancer, Small Intestine Cancer, Splenic Marginal Zone Lymphoma, Squamous-cell Carcinoma of the Lung, Squamous Cell Carcinoma, Squamous Cell Skin Cancer, Stomach Cancer, Surface Epithelial-Stromal Tumor, T-cell Prolymphocytic Leukemia, Testicular Cancer, Thoracic and Respiratory Cancer, Thymoma, Thymic Carcinoma, Thyroid Cancer, Transitional Cell Cancer, Transitional Cell Canceradenoid Cystic Carcinoma, Tuberous Sclerosis Syndrome, Tubular Carcinoma, Turcot Syndrome, Unknown Primary Cancer, Unsorted Cancer, Ureter Cancer, Urethral Cancer, Uterine Cancer, Uterine Sarcoma, Uveal Melanoma, Vaginal Cancer, Visual Pathway and Hypothalamic Glioma, Von Hippel-Lindau (VHL) Syndrome, Vulvar Cancer, Wilms Tumor, and Xeroderma Pigmentosum.

In embodiments, an allogeneic, biomimetic nanovesicle (BioNV) comprising (a) a membrane-embedded chimeric antigen receptor (CAR) targeted against a cell surface marker, and (b) a conjugated and/or membrane-anchored targeting agent targeted against PD-L1 or PD-L2, wherein the agent targeted against PD-L1 or PD-L2 is atezolizumab, avelumab, or durvalumab, and wherein the CAR is not targeted against PD-1, PD-L1, or PD-L2 is provided.

In embodiments, an allogeneic, biomimetic nanovesicle (BioNV) comprising (a) a first membrane-embedded chimeric antigen receptor (CAR) targeted against a cell surface, wherein the first membrane-embedded CAR is not targeted against PD-L1 or PD-L2, and (b) at least a second membrane-embedded CAR targeted against PD-L1 or PD-L2 is provided.

In embodiments, an allogeneic, biomimetic nanovesicle (BioNV) comprising a bispecific, membrane-embedded chimeric antigen receptor (CAR) targeted against (a) a cell surface marker of a cancer cell; and (b) PD-L1 or PD-L2, and wherein the cell surface marker of the cancer cell is not PD-1, PD-L1, or PD-L2 is provided.

### DESCRIPTION OF THE DRAWINGS

FIG. 1A-1C depict non-limiting diagrammatic representations of T cells binding to normal cells. FIG. 1A depicts PD-1 on T cells (left) interacting with PD-L1/2 expressed on healthy cells (right) to prevent an immune response. FIG. 1B depicts blocking PD-1 via pembrolizumab or nivolumab on T cells, preventing its interaction with PD-L1/2 on cancer cells (upregulated) and healthy cells. Immune reactions can then be mounted, and cancer cells can be cleared; however, off-target effects on healthy cells may occur. FIG. 1C depicts PD-L1 on cancer cells is overexpressed in comparison to healthy cells. The over-expression of PD-1 on cells protects them form immune attack, contributing to T-cell exhaustion.

FIG. 2A-2G depict non-limiting diagrammatic representations of various BioNVs with tumor targeted CARs and anti-PD-L1/2 integral membrane antibodies and/or bispecific CARs. FIG. 2A depicts a BioNV with tumor targeted CARs and anti-PD-L1/2 integral membrane antibodies to target tumor cells. FIG. 2B depicts a BioNV with tumor targeted anti-PD-L1 specific CARs or anti-PD-L1/2 bispecific CARs. FIG. 2C depicts a BioNV with tumor targeted anti-PD-L1 specific CARs or anti-PD-L1/2 bispecific CARs with combination treatment with pembrolizumab or nivolumab. FIG. 2D depicts a BioNV with tumor targeted CARs in combination treatment with pembrolizumab or nivolumab. FIG. 2E depicts a BioNV with tumor targeted bispecific CARs in combination treatment with pembrolizumab or nivolumab. FIG. 2F depicts a BioNV with tumor targeted CARs in combination treatment with pembrolizumab or nivolumab. FIG. 2G depicts a BioNV with tumor targeted bispecific CARs in combination treatment with pembrolizumab or nivolumab.

FIG. 3 depicts a non-limiting diagrammatic representation of serial extrusion to produce BioNVs.

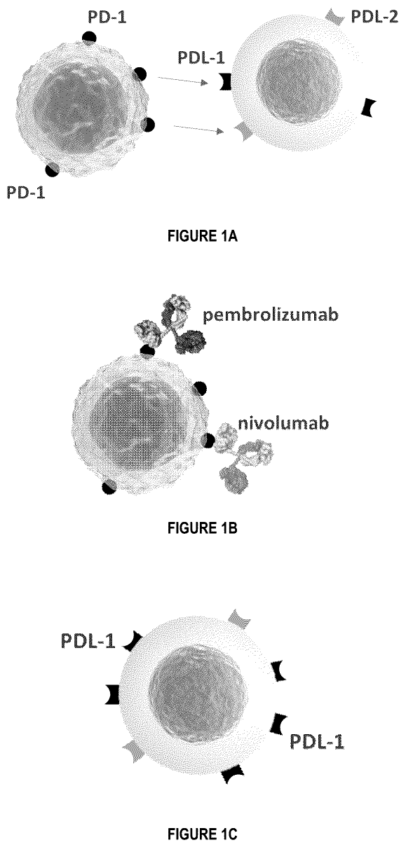

### DETAILED DESCRIPTION

The present disclosure relates to, in part, allogeneic, hypoimmunogenic cell-derived biomimetic nanovesicles (BioNVs) that find use in treating cancer with one or more surface-oriented chimeric antigen receptors (CARs) which recognize a single target, or multiple targets (i.e., bispecific), through a binding moiety for a desired biomarker/ligand. In embodiments, the designed BioNV is on the order of 20-1200 nm in size, far smaller in size than a traditional cell-based CAR-T/NK cell therapy. The BioNV can originate from a cell such as a stem cell, iPSC, reprogrammed pluripotent or multipotent cell, embryonic stem cell, mesenchymal stem cell, or a differentiated cell from any stem cell. The BioNV can also originate from a T cell, NK cell, macrophage, monocyte, etc. The plasma membrane-derived BioNVs retain the hypoimmunogenic properties of the modified cell from which they are derived. The modified cell can undergo genetic engineering focused on reducing or ablating (e.g., knock-out) of immunogenic cell surface markers or immunogenic molecules (e.g., MHC class I/II, HLA, T cell receptor (TCR), cytokine release syndrome (CRS), etc.) and/or expression or overexpression of immunoprotective cell surface markers (e.g., CD47, CD34, CD24, CD200, α-phagocytic, etc.). The binding moiety of the CAR can comprise all variations of an antibody construct, including for example, Fab, Fab′, Fab′-SH, F(ab′)2, scFv, diabody, nanobody, linear antibody, bispecific antibody, multi-specific antibody, chimeric antibody, humanized antibody, human antibody, and fusion proteins comprising the antigen-binding portion of an antibody, V_{H}H nanobodies, V_{NARS}, among other antibody formats, allowing BioNVs to target of any cancer cell of interest (targeting any type of cell surface biomarker). The BioNV can also encapsulate and deliver any cytotoxic protein, small molecule, biologic, nucleic acid, gene editing therapeutic payload, etc., of choice to the intended cellular targets to treat cancer. The BioNVs can include membrane-anchored and/or conjugated anti-cancer antibodies (e.g., pembrolizumab or nivolumab).

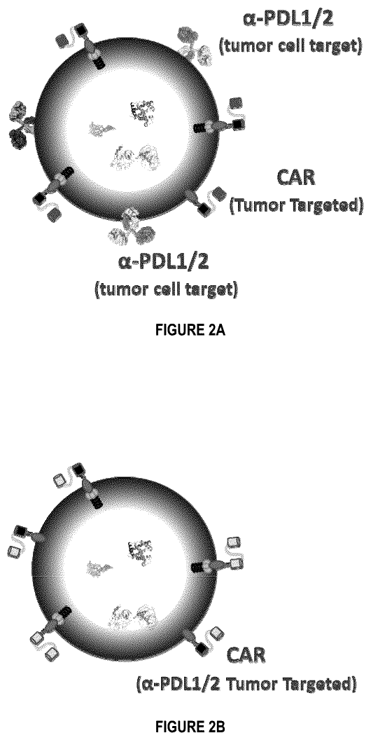

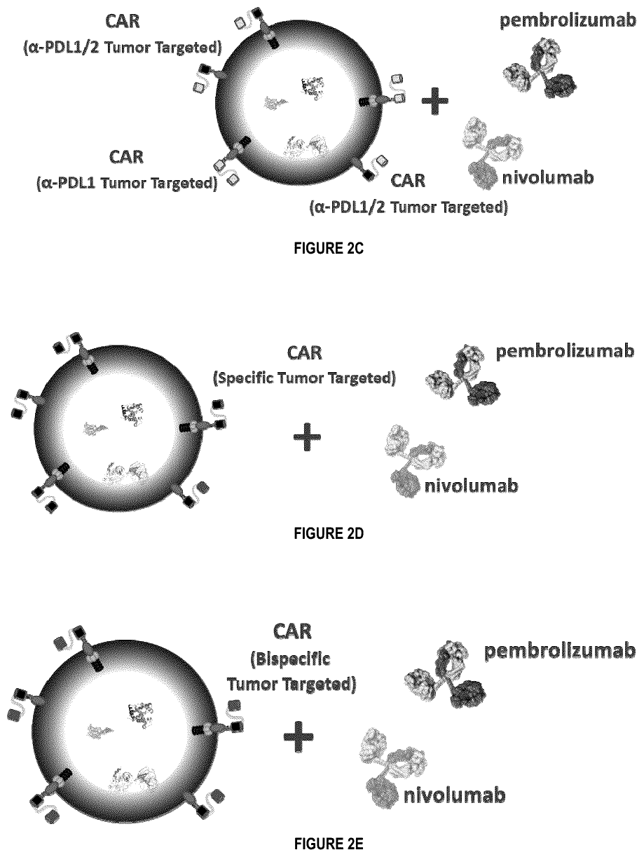

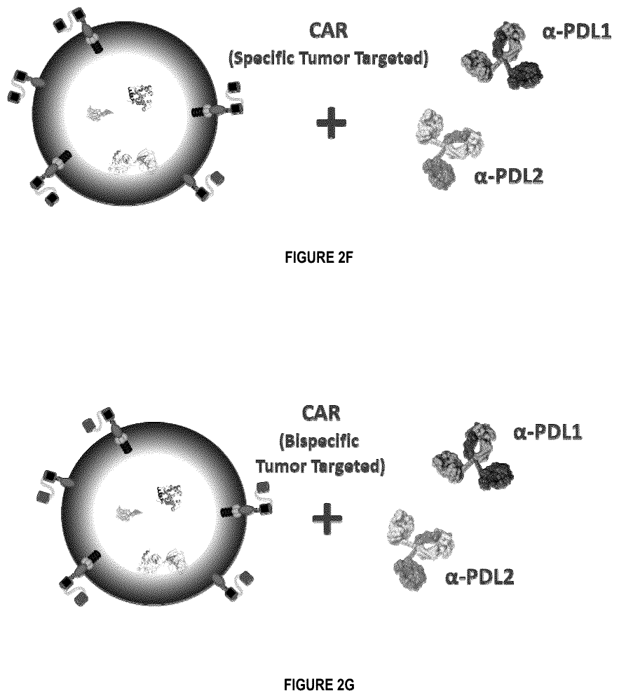

Moreover, the present disclosure relates to, in part, methods of treating and/or preventing cancer by administering compositions of a BioNV with a mono-specific and/or bispecific CAR (e.g., biomarker and anti-PD-L1 or anti-PD-L2), as depicted in FIG. 2B. In embodiments, the method of treatment involves targeting the cancer cell and providing a more specific block of PD-L1. In embodiments, the BioNV can be administered with pembrolizumab or nivolumab, as depicted in FIG. 2C-2G. In embodiments, the BioNV can include a tumor targeted anti-PD-L1-specific or anti-PD-L1/2-bispecific CAR (FIG. 2B-2C). In embodiments, the methods of treatment make use of a BioNV with a mono-specific and/or bispecific CAR and a conjugated and/or membrane-anchored antibody (e.g., pembrolizumab or nivolumab) (FIG. 2A). In embodiments, the present methods employ a construct or combination therapy as depicted in FIG. 2A, FIG. 2B, FIG. 2C, FIG. 2D, FIG. 2E, FIG. 2F, and/or FIG. 2G.

In embodiments, it is undesirable to have anti-PD-1 molecules on the BioNV because this could divert the CAR from the cancer cell to a T cell and/or potentially harm or kill the T cell. However, in embodiments, anti-PD-1 can be included as either a whole protein construct or as a minimal PD-L1/PD-L2 interactive domain of PD-1. In embodiments, this is engineered with a transmembrane domain so as to integrate into the BioNV lipid layer. In embodiments, the CAR (either mono- or bispecific) can be rapidly changed to target mutants as they arise. Biomarker variability is likely due to DNA repair mechanisms and drug selection and/or immunological selection pressures within the host. In embodiments, the methods of treatment use BioNVs that originate from a cytotoxic cell, allowing the BioNV to deliver anti-cancer molecules (e.g., cytokines, perforins, granzymes, etc.) to the tumor in a more controlled manner than a whole cell would. In embodiments, these molecules can be released from the BioNV and exhibit vastly improved penetration into the TME compared to traditional LNPs or whole cells. In embodiments, the scFV region of the CAR targets either PD-L1 or PD-L2, while the other scFV region (on the same CAR) recognizes a specific marker that is unique to the cancer, allowing for targeting both the cell for perforin/granzyme release to kill the cell and blocking PD-L1/PD-L2 from the PD-1 receptor on the host T cell, thereby preventing the cancer cell from immunological evasion. In embodiments, co-administration of pembrolizumab or nivolumab acts to bind to the PD-1 on the T-cell, further enhancing the effect.

Methods of Treating, Preventing, and/or Ameliorating Cancer

In aspects, the present disclosure includes methods of treating, preventing, and/or ameliorating a cancer comprising administering to a subject in need thereof, (i) a therapeutically effective amount of a biomimetic nanovesicle (BioNV) comprising a membrane-embedded chimeric antigen receptor (CAR) targeted against a cell surface marker and (b) a PD-1, PD-L1 and/or PD-L2 inhibiting agent; or (ii) a therapeutically effective amount of a BioNV comprising a membrane-embedded CAR targeted against a cell surface marker, wherein the subject is undergoing treatment with a PD-1, PD-L1, and/or PD-L2 inhibiting agent.

In aspects, the present disclosure includes methods of treating, preventing, and/or ameliorating a cancer comprising administering to a subject in need thereof (i) a therapeutically effective amount of a BioNV comprising (a) a membrane-embedded CAR targeted against a cell surface marker, and (b) a conjugated and/or membrane-anchored PD-L1 and/or PD-L2 inhibiting agent, or (ii) a therapeutically effective amount of a BioNV comprising (a) a membrane-embedded bispecific CAR targeted against a first cell surface marker and a second cell surface marker, and (b) a conjugated and/or membrane-anchored PD-L1 and/or PD-L2 inhibiting agent.

In aspects, the present disclosure includes methods of treating, preventing, and/or ameliorating a cancer comprising administering to a subject in need thereof (i) a therapeutically effective amount of a BioNV comprising a bispecific CAR targeted against a first cell surface marker and either PD-L1 or PD-L2, and wherein the first cell surface marker is not PD-1, PD-L1, or PD-L2; or (ii) a therapeutically effective amount of a BioNV comprising (a) a bispecific CAR targeted against a first cell surface marker and either PD-L1 or PD-L2, and wherein the first cell surface marker is not PD-1, PD-L1, or PD-L2, and (b) a PD-1, PD-L1, and/or PD-L2 inhibiting agent; or (iii) a therapeutically effective amount of a BioNV comprising a bispecific CAR targeted against either PD-L1 or PD-L2 and a first cell surface marker, wherein the subject is undergoing treatment with a PD-1, PD-L1, and/or PD-L2 inhibiting agent, and wherein the first cell surface marker is not PD-1, PD-L1, or PD-L2.

In aspects, the present disclosure includes methods of treating, preventing, and/or ameliorating a cancer comprising administering to a subject in need thereof a therapeutically effective amount of a BioNV comprising (a) a bispecific CAR targeted against a first cell surface marker and a second cell surface marker, and (b) a PD-1, PD-L1, and/or PD-L2 inhibiting agent.

In aspects, the present disclosure includes methods of treating, preventing, and/or ameliorating a cancer comprising administering to a subject in need thereof a therapeutically effective amount of a BioNV comprising a bispecific CAR targeted against a first cell surface marker and a second cell surface marker, wherein the subject is undergoing treatment with a PD-1, PD-L1, and/or PD-L2 inhibiting agent.

In embodiments, the CAR (mono- or bispecific) is targeted against a first cell surface marker and/or a second cell marker selected from Tables 1 and/or 2. In embodiments, the bispecific CAR is targeted against a first cell surface marker selected from Tables 1 or 2 and either PD-L1 or PD-L2.

TABLE 1
Illustrative checkpoint biomarkers and
ligands of checkpoint biomarkers.

| Biomarker | Illustrative Function |
|---|---|
| CTLA-4 | Immune checkpoint |
| PD-L1 | Suppress adaptive immune system |
| PD-L2 | Immune checkpoint receptor ligand |
| B7-H3 | Immune checkpoint |
| B7-H4 | Immune co-inhibitory molecule |
| BTLA | Immune regulatory receptor |
| HVEM | Receptor binding TNF receptor associated factor |
| | molecules |
| TIM3 | Immune response regulator |
| GAL9 | Induces T-cell death |
| LAG3 | Immune checkpoint |
| VISTA | Immune regulatory receptor |
| KIR | Regulate immune tolerance |
| 2B4 | Regulate NK cells |
| CD160 | Checkpoint inhibitor |
| CGEN-15049 | Immune checkpoint |
| CHK1 | Initiate DNA damage response |
| CHK2 | Regulates cell division |
| A2aR | Signals adenosine |
| B-7 family ligands | Regulate immune response |

TABLE 2
Illustrative cancer cell biomarkers.

| Biomarker | Illustrative Function |
|---|---|
| AFP (alpha-feroprotein) | Hepatocellular carcinoma (HCC) |
| Mesothelin | mesothelioma |
| ER (estrogen receptor) | Breast cancer |
| PR (progesterone | Breast cancer |
| receptor) | |
| HER-2/neu | Breast cancer |
| EGFR (epidermal growth | non-small-cell lung cancer |
| factor receptor) | |
| KRAS | non-small-cell lung cancer, colorectal cancer |
| UGT1A1 | colorectal cancer |
| c-KIT | Blood, gastric, skin cancers |
| CD30 | lymphomas |
| PDGFR (platelet derived | Gastrointestinal cancer |
| growth factor receptor) | |
| TEM8 (tumor endothelial | lung, breast, and colorectal cancer |
| marker 8) | |
| EIIIB | Various solid tumors |
| CA-125 | Ovarian cancer |
| CD2 | Adhesion between T cells and other cell types |
| | and T-cell activation |
| CD9 | Renal cell carcinoma |
| CD11 b | Various tumors |
| CD18 | Various tumors |
| CD19 | lymphomas |
| CD20 | lymphomas |
| CD21 | lymphomas |
| CD22 | endometrial cancer, intestinal cancer, skin |
| | cancer, leukemia |
| CD24 | Regulation of B-cell proliferation and |
| | differentiation |
| CD30 | lymphomas |
| CD31 | Angiosarcoma, Cell adhesion. Plays a key |
| | role in leukocyte trafficking across |
| | endothelium |
| CD32a | Dormant T cells harboring viruses |
| CD38 | Cell adhesion and signal transduction |
| CD41 | Ovarian cancer |
| CD42b | lymphomas |
| CD43 | Breast, colon, gastric, lung cancers |
| CD44 | Cell adhesion and migration |
| CD48 | Cell adhesion and T-cell costimulation |
| CD51 | Colorectal cancer |
| CD54 | Colorectal cancer, Cell adhesion, lymphocyte |
| | activation, and migration |
| CD61 | Breast, ovarian cancer |
| CD62P | Platelet activation |
| CD63 | Lung cancer |
| CD66a | Neutrophil activation, homophilic and |
| | heterophilic adhesion |
| CD66b | Cell adhesion and neutrophil activation |
| CD66c | Cell adhesion and neutrophil activation |
| CD66e | Homophilic and heterophilic adhesion. May |
| | play a role in the metastasis of cancer cells |
| CD74 | MHC class II antigen processing |
| CD87 | Cell chemotaxis and adhesion |
| CD96 | Involved in adhesive interactions of activated |
| | T cells and NK cells |
| CD98 | Cell activation and cell aggregation |
| CD105 | Angiogenesis. Regulatory component of |
| | TGF-beta receptor complex. Modulates |
| | cellular response to TGF beta 1 |
| CD106 | Leukocyte adhesion. Transmigration and |
| | costimulation of T cells. |
| CD107a | Degranulation marker |
| CD111 | Entry mediator for several herpes viruses |
| | (HSV1, HSV2, PRV, BHV1). Believed to be |
| | involved in cell adhesion mechanisms |
| CD112 | Intracellular adhesion, component of adherins |
| | junctions. Receptor for herpes simplex virus |
| CD117 | Crucial for development of gonadal and |
| | pigment stem cells |
| CD126 | Receptor for IL-6 |
| CD130 | Transmits activities of IL-6, IL-11, LIF |
| | and CNF |
| CD138 | Cell proliferation, cell migration, and |
| | cell-matrix interactions |
| CD140b | Cell proliferation, differentiation and survival |
| CD143 | Converts angiotensin I to angiotensin II by |
| | release of terminal His-Leu, which results |
| | in an increase of the vasoconstrictor activity |
| | of angiotensin. Cell adhesion. |
| CD144 | Cell adhesion. |
| CD146 | Cell adhesion. Involved in heterophilic cell |
| | to cell interactions |
| CD147 | Cell adhesion and T-cell activation |
| CD166 | Mediates cell adhesion by binding to CD6 for |
| | intrathymic T-cell development |
| CD171 | Multidomain cell adhesion molecule required |
| | for normal neurohistogenesis |
| CD202b | Signaling, migration and adhesion |
| CD208 | Dendritic cell maturation marker, involved in |
| | cell proliferation |
| CD221 | Receptor for IGF-I and IGF-II. Mediates |
| | mitogenic and anti-apoptotic signals |
| CD227 | Cell adhesion and signaling |
| CD239 | Laminin alpha-5 receptor |
| CD309 | Activation of this receptor by VEGF plays a |
| | role in angiogenesis, development, wound |
| | healing, cell proliferation, migration and |
| | differentiation |
| CD321 | Plays a role in epithelial tight junction |
| | formation, regulation of leukocyte |
| | erythrocytes. migration, epithelial |
| | barrier maintenance and platelet activation |
| CD324 | Calcium-dependent cell adhesion protein |
| CD326 | A homotypic calcium-independent T-cell |
| | adhesion molecule |
| CD333 | Cell-surface receptor for fibroblast growth |
| | factors |
| CD340 | Binds tightly to other ligand-bound EGF |
| | receptor family members to form a |
| | heterodimer, stabilizing ligand binding |
| | and enhancing kinase-mediated activation |
| | of downstream signaling pathways |
| CLEC-2 | Hepatocellular and gastric cancers |
| CD147 | Bladder cancer |
| GPVI | Contributes to thrombosis in cancer |

In embodiments, two or more biomarkers (or two or more epitopes of a single biomarker) can be targeted by a BioNV with a bispecific CAR, or multiple monospecific CARs. In embodiments, some biomarkers are found in multiple types of cancers and BioNVs can be targeted against multiple cancer types. In embodiments, mutant biomarkers can be selected if resistance arises to the primary biomarker target on a cancer cell, for example, as biomarkers on cancer cells have a tendency to mutate or become repressed. In embodiments, the CAR construct (all variations thereof) can be altered to match the mutation. In embodiments, the mutation present in the cancer is predictable, so the constructs can be made ahead of time, for example, SCN4A mutations can be used as predictors for check point inhibitor treatment regimens (Lin et al., “Potential Predictive Value of SCN4A Mutation Status for Immune Checkpoint Inhibitors in Melanoma.” Biomedicine & Pharmacotherapy. 2020). In embodiments, secondary biomarkers can be targeted in a follow-on treatment, for example, studies have also been conducted to target CD-22 in cells that have lost CD-19 expression.

In embodiments, the BioNV binds at least a first cell and at least a second cell to initiate an anti-cancer response. In embodiments the first cell is a cancer cell, and the second cell is an immune cell. In embodiments, the first cell and the second cell can both be of the same or similar cell type (e.g., both tumor cells).

In embodiments, the PD-1 inhibiting agent is an antibody targeted against PD-1, or pembrolizumab, nivolumab, or cemiplimab. In embodiments, the PD-L1 and/or PD-L2 inhibiting agent is atezolizumab, avelumab, or durvalumab.

In embodiments, the BioNV originates from a modified cell such as, without limitation, a stem cell, an induced pluripotent stem cell (iPSC), a reprogrammed pluripotent or multipotent cell, an embryonic stem cell, a mesenchymal stem cell, or a differentiated cell which originates from any modified cell thereof. In embodiments, the modified cell is an iPSC. In embodiments, the modified cell is a T cell, helper T cell, T-memory cell, or NK cell. In embodiments, the modified cell is a macrophage. In embodiments, the modified cell is a monocyte.

In embodiments, the allogeneic and hypoimmunogenic properties of the modified cell (e.g., derived from iPSCs) are created by knocking-out, silencing, inactivating, blocking or otherwise negating the expression, transcriptional efficiencies, and/or activity of one or more immunogenic molecules. In embodiments, the modified cell substantially lacks one or more MHC class I proteins, MHC class II proteins, HLA proteins, TCR proteins, and/or CRS proteins.

In embodiments, the reducing or ablating the expression and/or activity of one or more immunogenic proteins includes a β2-macroglobulin (B2M) gene disruption and/or a disruption that reduces or ablates MHC class I protein expression and/or activity, such as in the case for CD8+ T cell lineages. In embodiments, the reducing or ablating the expression and/or activity of one or more immunogenic proteins includes a CIITA gene disruption and/or a disruption that reduces or ablates MHC class II protein expression and/or activity, such as in the case of CD4+ T cell lineages. Without wishing to be bound by theory, these proteins contribute to the human leukocyte antigen (HLA) immunogenicity that requires HLA allele matching in the donor-recipient for treatment by cell-based therapies. In embodiments, allogeneic and/or hypoimmunogenic properties are achieved by reducing or ablating the expression and/or activity of the genes encoding the T cell receptor (TCR) proteins including, for example, the α and β chains (as in the case of αβ T cells) or the γ and δ chains (as in the case of γδ T cells) forming the ligand-binding site and the signaling modules CD3δ, CD3γ, CD3ε, and CD3ζ. In embodiments, this is performed to reduce extraneous T cell receptor types other than those of the CAR cassette and further improve the homogeneity of the CAR of interest and reduce off-target effects in BioNV formation.

In embodiments, the modified cell comprises a β2-macroglobulin (B2M) gene disruption, or a gene disruption that disrupts MHC class I expression. In embodiments, knocking-out the B2M genes reduces the number of potential doses to be administered due to the risk of preventing long term acceptance of the BioNVs by the recipient, such as what has been observed in the whole cell-based approaches described above. To overcome this issue, in embodiments, the HLA-E or HLA-G gene remains intact, allowing the immune system to adapt to the resulting BioNV. In embodiments, the HLA-A, HLA-B, HLA-C, HLA-F, HLA-E or HLA-G (but not both) are knocked out sequentially.

In embodiments, the modified cell has reduced or ablated expression of an HLA-A gene and/or reduced or ablated HLA-A protein expression and/or activity. In embodiments, the modified cell has reduced or ablated expression of an HLA-B gene and/or reduced or ablated HLA-B protein expression and/or activity. In embodiments, the modified cell has reduced or ablated expression of an HLA-C gene and/or reduced or ablated HLA-C protein expression and/or activity. In embodiments, wherein the modified cell has reduced or ablated expression of an HLA-E or HLA-G gene and/or reduced or ablated HLA-E or HLA-G protein expression and/or activity. In embodiments, the modified cell has reduced or ablated expression of an HLA-F gene and/or reduced or ablated HLA-F protein expression and/or activity.

In embodiments, the modified cell has reduced or ablated expression of a CIITA gene and/or reduced or ablated MHC class II protein expression and/or activity. In embodiments, allogeneic iPSCs have their MHC Class I and MHC Class II complexes disrupted by knocking out critical proteins involved in their expression, for example, β2-macroglobulin (B2M) which is a serum protein found in association with the MHC class I heavy chain on the surface of nearly all nucleated cells that is involved in the presentation of peptide antigens to the immune system. In embodiments, the allogeneic iPSCs have their CIITA gene disrupted, which is the Master Control Transcription Factor that regulates the expression of all MHC II genes. In embodiments, allogeneic iPSCs have their CIITA gene disrupted, which is the master control transcription factor that regulates the expression of all MHC II genes so that a resulting differentiated cell line (e.g., DCs, mononuclear phagocytes, endothelial cells, thymic epithelial cells, B cells, etc.) does not express or has reduced expression of MHC class II proteins.

In embodiments, the modified cell has reduced or ablated expression of a T cell alpha constant (TRAC) gene and/or reduced or ablated TRAC protein expression and/or activity. In embodiments, the modified cell has reduced or ablated expression of a T cell beta constant (TRBC) gene and/or reduced or ablated TRBC protein expression and/or activity.

In embodiments, the modified cell has reduced or ablated expression of a PD-1 gene and/or reduced or ablated PD-1 protein expression and/or activity.

CRS is a major concern with whole cell therapies, where despite engineered hypoimmunogenicity, effector functions and other consequences of interaction with cells post-infusion can result in the release of biomolecules that result in a systemic inflammatory syndrome characterized by fever, multiple organ dysfunction, etc. In embodiments, the modified cell is engineered to disrupt one or more proteins that contribute to CRS. In embodiments, the modified cell has reduced or ablated expression and/or activity (e.g., knock-out or silencing) of CRS-related cytokines.

In embodiments, the modified cell has reduced or ablated expression of an IL-4 gene and/or reduced or ablated IL-4 protein expression and/or activity. In embodiments, the modified cell has reduced or ablated expression of an IL-6 gene and/or reduced or ablated IL-6 protein expression and/or activity. In embodiments, IL-6 knock-out prevents undesirable IL-6 packaging into the BioNV and reduces the BioNV's contribution to a localized (and concentrated due to biomarker targeting) and/or potentially systemic CRS events. In embodiments, the modified cell has reduced or ablated expression of an IL-10 gene and/or reduced or ablated IL-10 protein expression and/or activity. In embodiments, the modified cell has reduced or ablated expression of an IL-16 gene and/or reduced or ablated IL-16 protein expression and/or activity. In embodiments, the reduction or ablation of interleukins decreases the likelihood of CRS.

Serine proteinase inhibitor B9 (SerpinB9) is a member of the serine protease inhibitor superfamily. SerpinB9 has been reported to protect cells from the immune-killing effects of granzyme B. In embodiments, such as where the BioNV is not designed to delivery granzyme, the modified cell that the BioNV is derived from expresses or has increased expression of SerpinB9. In embodiments, such as where the BioNV is designed to deliver granzyme, the modified cell that the BioNV is derived from has SerpinB9 knocked-out and/or silenced. In embodiments, the modified cell has reduced or ablated expression and/or activity of a SerpinB9 gene and/or reduced or ablated SerpinB9 protein expression and/or activity.

In embodiments, the overexpression of SerpinB9 sequesters the function of granzyme B which is related to immunostimulatory responses, such as apoptosis of a targeted and/or diseased cell. In embodiments, granzyme B is inhibited in activated lymphocytes, NK cells, macrophages, and follicular DCs, among other cell types. In embodiments, e.g., where a BioNV that is intended to deliver a non-granzyme payload, for example a gene editing payload, the modified cell can express and/or have increased expression of SerpinB9.

In embodiments, methods of treating, preventing, and/or ameliorating cancer use BioNVs which originate from cells modified to be hypoimmunogenic due to expression or increased expression of one or more immunoprotective proteins. In embodiments, the modified cell expresses or has increased expression of a CD34 gene and/or gene product. In embodiments, the modified cell expresses or has increased expression of a CCL2 gene and/or gene product. In embodiments, the modified cell expresses or has increased expression of a PD-L1 gene and/or gene product, wherein the modified cell is not activated, e.g., and where the resultant BioNV does not encapsulate perforin and/or granzyme. In embodiments, the modified cell has reduced or ablated expression of a PD-L1 gene and/or gene product, and wherein the modified cell is activated, e.g., and where the resultant BioNV encapsulates perforin and/or granzyme. In embodiments, the modified cell expresses or has increased expression of a H2-M3 gene and/or gene product.

In embodiments, the modified cell expresses or has increased expression of a CD47 gene and/or gene product. Exosomes and cell-derived vesicles (CDVs) are readily cleared from the body by macrophages through phagocytosis. Phagocytosis greatly impacts the therapeutic value and efficacy of CDVs. To prevent macrophage depletion of BioNVs but without wishing to be bound by theory, in embodiments, the BioNV is CD47 tagged on the surface. A CD47tg (tag) provides a “do not eat me” signal which, in embodiments, increases the half-life and serum stability of the BioNV in the subject. In embodiments, the molecular CD47 isoform 2 (the isoform that interacts with the SIRPα receptor on a macrophage) is engineered into the modified cell (e.g., iPSC cell line). Without the CD47tg, the BioNV half-life would be diminished due to phagocytosis inhibition, resulting in the need for higher and/or more frequent doses. In embodiments, prevention of the potential inhibitory phenotypes of CD47 expression across cells is done via interference with the inhibitory mechanism of action of the series of microRNAs on the 3′UTR of the CD47 gene by deleting this region in stable constructs or by eliminating/inhibiting the expression of the microRNAs. In embodiments, this can resolve inhibitory issues caused by the microRNAs across differentiated cell subsets.

In embodiments, the modified cell overexpresses CD24. CD24 is a sialoglycoprotein expressed on mature granulocytes and B-cells and is also an anti-phagocytic protein. CD24 prevents phagocytosis through interactions with Siglec-G/10 on macrophages. In embodiments, the modified cell expresses or has increased expression of a CD24 gene and/or gene product. In embodiments, the modified cell expresses or has increased expression of a chimeric CD24/CD47 gene and/or gene product. In embodiments, the modified cell expresses chimeric CD24/CD47 with a tethered transmembrane domain. In embodiments, the domains of CD47 isoform 2 and CD24 can be either separately expressed or tethered to form a bilobed, chimeric protein. In embodiments, the modified cells are iPSCs are from fibroblasts, not ABO cells.

In embodiments, the modified cell expresses or has increased expression of a chimeric CD200 gene and/or gene product. In embodiments, CD200 tags minimize phagocytosis by macrophages and prevent the activation of granulocytes. In embodiments, the modified cell does not express CD200 when it is not desirable to prevent granulocytes, for example in the TME, as activation of granulocytes would complement the mechanism of action of a BioNV designed to release granzymes and perforins. However, in embodiments, if a CD47 or CD24 tag is used, or a CD24/CD47 chimeric, bilobed protein tag (each prevents phagocytosis) in combination with overexpressed H2-M3 (which dampens the NK response) is used, stability can be achieved without CD200, while allowing adequate BioNV clearance. In embodiments, where granzymes and perforins are not a therapeutic biomolecule of choice, CD200 can be expressed to prevent the activation of granulocytes, while eliminating a CD47 tag or a CD24 tag, but not both tags. In embodiments, the modified cell expresses or has increased expression of a chimeric CD24/CD200 gene and/or gene product or a chimeric CD47/CD200 gene and/or gene product.

In embodiments, the modified cell (or differentiated cell therefrom) does not express all three of CD47, CD24, and CD200. In embodiments, the modified cell that the BioNV is derived from is engineered such that BioNVs are stabilized, but not to a degree where the BioNVs are resistant to being cleared from the body. A BioNV that is too stable could eventually trigger a humoral response, resulting in limiting the number of doses or treatments that can be administered.

In embodiments, the modified cell expresses or has increased expression of a chimeric CTLA-4 gene and/or gene product. In embodiments, the modified cell expresses or has increased expression of a chimeric MFG-E8 gene and/or gene product. In embodiments, the modified cell expresses or has increased expression of an NCAM gene and/or gene product. In embodiments, the modified cell expresses or has increased expression of a chimeric α-phagocytic integrin gene and/or gene product.

In embodiments, the modified cell expresses or has increased expression of an antibody or antibody format molecule which targets an IL-6 surface receptor (anti-IL-6R). In embodiments, an iPSC cell line for generating anti-cancer BioNVs has an anti-IL-6R antibody engineered into the cell line. In embodiments, BioNVs carry α-IL-6R, thereby blocking the signaling pathway on localized immune cells in the tumor environment from becoming activated.

In embodiments, the modified cell expresses or has increased expression of a FasL gene and/or gene product. In embodiments, the modified cell does not overexpress a FasL gene and/or gene product. In embodiments, the overexpression of FasL in the modified cell is avoided because an enrichment of naturally expressed levels of FasL is observed in the membranes of BioNVs after processing, e.g., via serial extrusion. Too high of concentrations of FasL can be counter-productive and prevent the recruitment of T-cells to the solid tumor and/or cause premature T-cell death.

In embodiments, the modified cells can express one or more fusion proteins of one or more portions of any immunoprotective protein herein. For example, in embodiments, constructs can be made where the appropriate portion of a ligand of choice is tethered to a transmembrane domain so that the entire protein (e.g., intracellular signaling domains) is not necessary. In embodiments, constructs can be made where the biologically relevant portion of two or more proteins are tethered together and/or to a transmembrane domain.

In embodiments, the modified cell has reduced or ablated expression and/or activity of one or more immunogenic proteins, such as proteins that result in an immune response in the subject, donor-recipient mismatch, HLA alloimmunity, inflammation, CRS, and the like, such as MHC class I proteins, MHC class II proteins, HLA proteins, TCR proteins, CRS proteins, etc. In embodiments, the modified cell has reduced or ablated expression and/or activity of 3 or more immunogenic proteins, 4 or more immunogenic proteins, 5 or more immunogenic proteins, 6 or more immunogenic proteins, 7 or more immunogenic proteins, 8 or more immunogenic proteins, 9 or more immunogenic proteins, 10 or more immunogenic proteins, 11 or more immunogenic proteins, or 12 or more immunogenic proteins.

In embodiments, the modified cell has expression or increased expression and/or activity of one or more immunoprotective proteins, such as proteins that result prevent or reduce an immune response in the subject, prevent or reduce premature clearance of the BioNV in the subject, prevent or reduce phagocytosis, confer barrier-crossing functionality, and the like, such as CD47, CD24, CD200, CD34, CCL2, H2-M3, MFG-E8, PD-L1 (for non-activated cells), CTLA-4, etc. In embodiments, the modified cell expresses or has increased expression of 3 or more immunoprotective proteins, 4 or more immunoprotective proteins, 5 or more immunoprotective proteins, 6 or more immunoprotective proteins, 7 or more immunoprotective proteins, 8 or more immunoprotective proteins, 9 or more immunoprotective proteins, or 10 or more immunoprotective proteins.

In embodiments, “increased expression,” and “increased expression and/or activity,” as used herein refers to an increase in expression and/or activity in the hypoimmunogenic cell and/or results BioNV in comparison to a native, or wild-type cognate cell. For example, in embodiments, the increased expression and/or activity of one or more biomolecules described herein can confer the hypoimmunogenic properties of an iPSC relative to an iPSC which does not have the same expression pattern or expression level of the protein. In embodiments, the “increased expression,” is due to a genetic amendment, such as a knock-in.

In embodiments, the modified cell is allogeneic. In embodiments, the modified cell does not cause an immune reaction in patients to which it or a BioNV derived therefrom is administered.

In embodiments, the modified cell undergoes specific processing before manufacture of the BioNVs, such as activation of the modified cell (e.g., via TCR/CD3, CD28, etc. to increase expression of granzyme, perforin, etc.), differentiation of the modified cell (e.g., from iPSCs into T cells, NK cells, macrophages, etc.), and/or activation of the CAR. In embodiments, the modified cell is differentiated prior to BioNV formation. In embodiments, the modified cell expresses the CAR by a regulatable expression element. In embodiments, the CAR is activated prior to BioNV formation. In embodiments, the CAR is activated via its target, through another receptor, and/or a virus. In embodiments, the modified cell is expanded after engineering; any small-scale expansion or large-scale feeder system expansion methods known in the art can be used.

In embodiments, the type of cancers to be targeted drives the differentiation into a given cell line. In embodiments, BioNVs can be derived from activated macrophage/monocytes if brain cancers are being targeted. In embodiments, BioNVs carry over some phenotype properties from the cells from which they are derived (their parent cells). For example, in embodiments, macrophage/monocytes readily cross the BBB and this property (which is likely membrane receptor driven) can be transferred to the BioNV. In embodiments, BioNVs can be generated from Tumor Infiltrating Lymphocytes (TILs) for dense tumors.

In embodiments, the BioNV is formed from the modified cell, or the differentiated cell thereof (e.g., after activation), by sonication, adaptive focused acoustics technology, French press, extrusion, serial extrusion, cell lysis by detergent, enzymatic rupture of cells (e.g., trypsinization), and/or electroporation. In embodiments, the BioNV is formed from the modified cell, or the differentiated cell thereof, by serial extrusion.

BioNVs, in embodiments, are about 10 nm to 1200 nm in size. In embodiments, BioNVs are about 10 nm in size, about 20 nm in size, about 30 nm in size, about 40 nm in size, about 50 nm in size, about 60 nm in size, about 70 nm in size, about 80 nm in size, about 90 nm in size, about 100 nm in size, about 120 nm in size, about 140 nm in size, about 160 nm in size, about 180 nm in size, about 200 nm in size, about 300 nm in size, about 400 nm in size, about 500 nm in size, about 600 nm in size, about 700 nm in size, about 800 nm in size, about 900 nm in size, about 1000 nm in size, about 1100 nm in size, or about 1200 nm in size. In embodiments, BioNVs range in size from about 10 nm to 100 nm in size, about 100 nm to 200 nm in size, about 200 nm to 500 nm in size, or about 500 nm to 1200 nm in size.

In embodiments, in methods of treating, preventing, and/or ameliorating cancer as described herein, the size of the BioNV is matched to the cancer type, tumor type, progression of disease (e.g., metastasis, spread, pathology, etc.), tissue type, etc. In embodiments, methods of treating very dense/gummy tumor types, such as retinoblastoma, utilize BioNVs of low size (e.g., less than about 500 nm in size, less than about 400 nm in size, less than about 300 nm in size, less than about 200 nm in size, less than about 100 nm in size, or less than about 50 nm in size) for higher penetration. In embodiments, methods of treating loose tumor types, such as lung cancers, utilize larger BioNVs (e.g., greater than about 500 nm in size, greater than about 600 nm in size, greater than about 700 nm in size, greater than about 800 nm in size, greater than about 900 nm in size, or greater than about 1000 nm in size).

In embodiments, the BioNV substantially lacks protein and/or activity of HLA-A, HLA-B, HLA-C, HLA-F, CIITA, IL-6, TRAC, TRBC, and one of either HLA-E or HLA-G.

In embodiments, the BioNV substantially lacks protein and/or activity of HLA-A, HLA-B, HLA-C, HLA-F, CIITA, IL-6, TRAC, TRBC, SerpinB9, and one of either HLA-E or HLA-G.

In embodiments, the BioNV substantially lacks protein and/or activity of HLA-A, HLA-B, HLA-C, HLA-F, CIITA, IL-6, TRAC, TRBC, SerpinB9, CD200, one of either HLA-E or HLA-G, and one or more of IL-4, IL-10, and IL-16.

In embodiments, the BioNV has a membrane-embedded α-phagocytic integrin, CCL2, H2-M3, FasL, MFG-E8, anti-IL-6R antibody or antibody format, and PD-L1 (in BioNVs from non-activated cells) and/or CTLA-4, and one of either CD24, CD47, CD200, chimeric CD24/CD47, chimeric CD24/CD200, and chimeric CD47/CD200, or two of either CD24, CD47, and CD200.

In embodiments, the BioNV has a membrane-embedded α-phagocytic integrin, CCL2, H2-M3, FasL, MFG-E8, anti-IL-6R antibody or antibody format, SerpinB9, and PD-L1 (in BioNVs from non-activated cells) and/or CTLA-4, and one of either CD24, CD47, CD200, chimeric CD24/CD47, chimeric CD24/CD200, and chimeric CD47/CD200, or two of either CD24, CD47, and CD200.

In embodiments, the BioNV has a membrane-embedded CD200 protein and substantially lacks protein of either CD24 or CD47. In embodiments, the BioNV substantially lacks protein and/or activity of SerpinB9 and CD200.

In embodiments, the BioNV CAR comprises an antibody or antibody format selected from one or more of a monoclonal antibody, polyclonal antibody, antibody fragment, VNAR, VHH, Fab, Fab′, Fab′-SH, F(ab′)2, Fv, single chain Fv (scFv), diabody, nanobody, linear antibody, bispecific antibody, multi-specific antibody, chimeric antibody, humanized antibody, human antibody, or fusion protein comprising the antigen-binding portion of an antibody. In embodiments, the antibody format is a scFV. In embodiments, the CAR comprises a viral ligand.

In embodiments, the CAR includes a transmembrane domain derived from CD28, CD3ζ, CD4, CD8α, ICOS, or fragment and/or combination thereof. In embodiments, the CAR includes an intracellular domain further comprising an intracellular signaling domain of a CD3ζ-chain and/or one or more co-stimulatory molecules, optionally selected from CD28, 4-1BB, ICOS, CD27, and OX40. In embodiments, the CAR lacks an intracellular domain and/or co-stimulatory domain.

In embodiments, CAR targeting is against a cancer-specific antigen, for example and without limitation, CAR targeting AFP (alpha-ferroprotein) in methods of treating hepatocellular carcinoma (HCC); CAR targeting mesothelin in methods of treating mesothelioma; CAR targeting ER (estrogen receptor), PR (progesterone receptor), or HER-2/neu in methods of treating breast cancer; CAR targeting EGFR (epidermal growth factor receptor) in methods of treating non-small-cell lung cancer; CAR targeting KRAS in methods of treating non-small-cell lung cancer and/or colorectal cancer; CAR targeting UGT1A1 in methods of treating colorectal cancer; CAR targeting c-KIT in methods of treating blood, gastric, and/or skin cancers; CAR targeting CD30 in methods of treating lymphomas; CAR targeting PDGFR (platelet derived growth factor receptor) in methods of treating gastrointestinal cancer; CAR targeting TEM8 (tumor endothelial marker 8) in methods of treating lung, breast and colorectal cancer; CAR targeting EIIIB in methods of treating various solid tumors; CAR targeting CA-125 in methods of treating ovarian cancer; CAR targeting CD9 in methods of treating renal cell carcinoma; CAR targeting CD11b and/or CD18 in methods of treating various tumors; CAR targeting CD19, CD20, and/or CD21 in methods of treating lymphomas; CAR targeting CD22 in methods of treating endometrial cancer, intestinal cancer, skin cancer, and/or leukemia; CAR targeting CD24 in methods of treating B-cell cancers; CAR targeting CD30 in methods of treating lymphomas; CAR targeting CD32a in methods of treating cancer driven by dormant T cells harboring viruses; CAR targeting CD41 in methods of treating ovarian cancer; CAR targeting CD42b in methods of treating lymphomas; CAR targeting CD43 in methods of treating breast, colon, gastric, and/or lung cancers; CAR targeting CD51 and/or CD54 in methods of treating colorectal cancer; CAR targeting CD61 in methods of treating breast and/or ovarian cancer; CAR targeting CD63 in methods of treating lung cancer; CAR targeting CD66a/b/c/e in methods of treating the metastasis of cancer; CAR targeting CLEC-2 in methods of treating hepatocellular and/or gastric cancers; CAR targeting CD147 in methods of treating bladder cancer; CAR targeting GPVI in methods of treating thrombosis due to cancer. In embodiments, the CAR is targeted against a cancer-specific antigen of one or more targets in Table 1 and/or Table 2.

In embodiments, the BioNV encapsulates a payload; e.g., “lumen-loading”, or the ability of the BioNV to have a payload loaded into the lumen (space in the biomimetic nanovesicle). In embodiments, the payload is one or more of a gene editor, cytotoxic protein, biologic, nucleic acid, fusion protein, fluorescent protein, tracing dye, radionuclide, and/or small molecule. In embodiments, the payload is a therapeutic payload for a cancer type that the CAR is targeted against. In embodiments, the payload comprises one or more of an alkylating agent, an anthracycline, an antimetabolite, an anti-tumor antibiotic, an anti-tumor antibody or antibody format, a corticosteroid, a plant alkaloid, a topoisomerase inhibitor, and/or a checkpoint inhibitor.

In embodiments, the payload is selected from one or more of Abecma, Abemaciclib, Abiraterone Acetate, Abraxane, ABVD, ABVE, ABVE-PC, AC, Acalabrutinib, AC-T, Actemra, Adcetris, ADE, Ado-Trastuzumab Emtansine, Adriamycin, Afatinib Dimaleate, Afinitor, Akynzeo, Aldara, Aldesleukin, Alecensa, Alectinib, Alemtuzumab, Alimta, Aliqopa, Alkeran for Injection, Alkeran Tablets, Aloxi, Alpelisib, Alunbrig, Ameluz, Amifostine, Aminolevulinic Acid Hydrochloride, Amivantamab-vmjw, Anastrozole, Apalutamide, Aprepitant, Aranesp, Aredia, Arimidex, Aromasin, Arranon, Arsenic Trioxide, Arzerra, Asciminib Hydrochloride, Asparaginase Erwinia Chrysanthemi, Asparlas, Atezolizumab, Avapritinib, Avastin, Avelumab, Axicabtagene Ciloleucel, Axitinib, Ayvakit, Azacitidine, Azedra, Balversa, Bavencio, BEACOPP, Belantamab Mafodotin-blmf, Beleodaq, Belinostat, Belzutifan, Bendamustine Hydrochloride, Bendeka, BEP, Besponsa, Besremi, Bevacizumab, Bexarotene, Bicalutamide, BICNU, Binimetinib, Blenrep, Bleomycin Sulfate, Blinatumomab, Blincyto, Bortezomib, Bosulif, Bosutinib, Braftovi, Brentuximab Vedotin, Brexucabtagene Autoleucel, Breyanzi, Brigatinib, Brukinsa, BuMel, Busulfan, Busulfex, Cabazitaxel, Cablivi, Cabometyx, Cabozantinib-S-Malate, CAF, Calaspargase Pegol-mknl, Calquence, Campath, Camptosar, Capecitabine, Caplacizumab-yhdp, Capmatinib Hydrochloride, CAPOX, Carac, Carboplatin, CARBOPLATIN-TAXOL, Carfilzomib, Carmustine, Carmustine Implant, Casodex, CEM, Cemiplimab-rwlc, Ceritinib, Cerubidine, Cervarix (Recombinant HPV Bivalent Vaccine), Cetuximab, CEV, Chlorambucil, CHLORAMBUCIL-PREDNISONE, CHOP, Cisplatin, Cladribine, Clofarabine, Clolar, CMF, Cobimetinib Fumarate, Cometriq, Copanlisib Hydrochloride, COPDAC, Copiktra, COPP, COPP-ABV, Cosmegen, Cotellic, Crizotinib, CVP, Cyclophosphamide, Cyramza, Cytarabine, Dabrafenib Mesylate, Dacarbazine, Dacogen, Dacomitinib, Dactinomycin, Danyelza, Daratumumab, Daratumumab and Hyaluronidase-fihj, Darbepoetin Alfa, Darolutamide, Darzalex, Darzalex Faspro, Dasatinib, Daunorubicin Hydrochloride, Daunorubicin Hydrochloride and Cytarabine Liposome, Daurismo, Decitabine, Decitabine and Cedazuridine, Defibrotide Sodium, Defitelio, Degarelix, Denileukin Diftitox, Denosumab, Dexamethasone, Dexrazoxane Hydrochloride, Dinutuximab, Docetaxel, Dostarlimab-gxly, Doxil, Doxorubicin Hydrochloride, Doxorubicin Hydrochloride Liposome, Durvalumab, Duvelisib, Efudex, Eligard, Elitek, Ellence, Elotuzumab, Eloxatin, Eltrombopag Olamine, Elzonris, Emapalumab-Izsg, Emend, Empliciti, Enasidenib Mesylate, Encorafenib, Enfortumab Vedotin-ejfv, Enhertu, Entrectinib, Enzalutamide, Epirubicin Hydrochloride, EPOCH, Epoetin Alfa, Epogen, Erbitux, Erdafitinib, Eribulin Mesylate, Erivedge, Erleada, Erlotinib Hydrochloride, Erwinaze, Ethyol, Etopophos, Etoposide, Etoposide Phosphate, Everolimus, Evista, Evomela, Exemestane, Exkivity, 5-FU, 5-FU, Fam-Trastuzumab Deruxtecan-nxki, Fareston, Faslodex, FEC, Fedratinib Hydrochloride, Femara, Filgrastim, Firmagon, Fludarabine Phosphate, Fluoroplex, Fluorouracil Injection, Fluorouracil-Topical, Flutamide, FOLFIRI, FOLFIRI-BEVACIZUMAB, FOLFIRI-CETUXIMAB, FOLFIRINOX, FOLFOX, Folotyn, Fostamatinib Disodium, Fotivda, Fulphila, FU-LV, Fulvestrant, Fyarro, Gamifant, Gardasil (Recombinant HPV Quadrivalent Vaccine), Gardasil 9 (Recombinant HPV Nonavalent Vaccine), Gavreto, Gazyva, Gefitinib, Gemcitabine Hydrochloride, GEMCITABINE-CISPLATIN, GEMCITABINE-OXALIPLATIN, Gemtuzumab Ozogamicin, Gemzar, Gilotrif, Gilteritinib Fumarate, Glasdegib Maleate, Gleevec, Gliadel Wafer, Glucarpidase, Goserelin Acetate, Granisetron, Granisetron Hydrochloride, Granix, Halaven, Hemangeol, Herceptin Hylecta, Herceptin, HPV Bivalent Vaccine, Recombinant, HPV Nonavalent Vaccine, Recombinant, HPV Quadrivalent Vaccine, Recombinant, Hycamtin, Hydrea, Hydroxyurea, Hyper-CVAD, Ibrance, Ibritumomab Tiuxetan, Ibrutinib, ICE, Iclusig, Idamycin PFS, Idarubicin Hydrochloride, Idecabtagene Vicleucel, Idelalisib, Idhifa, Ifex, Ifosfamide, Interleukin-2 (recombinant), Imatinib Mesylate, Imbruvica, Imfinzi, Imiquimod, Imlygic, Infigratinib Phosphate, Infugem, Inlyta, Inotuzumab Ozogamicin, Inqovi, Inrebic, Interferon Alfa-2b, Intron A, Iobenguane I131, Ipilimumab, Iressa, Irinotecan Hydrochloride, Isatuximab-irfc, Istodax, Ivosidenib, Ixabepilone, Ixazomib Citrate, Ixempra, Jakafi, JEB, Jelmyto, Jemperli, Jevtana, Kadcyla, Kepivance, Keytruda, Kimmtrak, Kisqali, Koselugo, Kymriah, Kyprolis, Lanreotide Acetate, Lapatinib Ditosylate, Larotrectinib Sulfate, Lenalidomide, Lenvatinib Mesylate, Lenvima, Letrozole, Leucovorin Calcium, Leukeran, Leuprolide Acetate, Levulan Kerastik, Libtayo, Lisocabtagene Maraleucel, Lomustine, Loncastuximab Tesirine-lpyl, Lonsurf, Lorbrena, Lorlatinib, Lumakras, Lumoxiti, Lupron Depot, Lurbinectedin, Luspatercept-aamt, Lutathera, Lutetium (Lu 177-Dotatate), Lynparza, Margenza, Margetuximab-cmkb, Marqibo, Matulane, Mechlorethamine Hydrochloride, Megestrol Acetate, Mekinist, Mektovi, Melphalan, Melphalan Hydrochloride, Mercaptopurine, Mesna, Mesnex, Methotrexate Sodium, Methylnaltrexone Bromide, Midostaurin, Mitomycin, Mitoxantrone Hydrochloride, Mobocertinib Succinate, Mogamulizumab-kpkc, Monjuvi, MOPP, Moxetumomab Pasudotox-tdfk, Mozobil, MVAC, Mvasi, Myleran, Mylotarg, Nanoparticle Paclitaxel, Naxitamab-gqgk, Necitumumab, Nelarabine, Neratinib Maleate, Nerlynx, Netupitant, Neulasta, Neupogen, Nexavar, Nilandron, Nilotinib, Nilutamide, Ninlaro, Niraparib Tosylate Monohydrate, Nivestym, Nivolumab, Nplate, Nubeqa, Nyvepria, Obinutuzumab, Odomzo, OEPA, Ofatumumab, OFF, Olaparib, Omacetaxine Mepesuccinate, Oncaspar, Ondansetron Hydrochloride, Onivyde, Ontak, Onureg, Opdivo, OPPA, Orgovyx, Osimertinib Mesylate, Oxaliplatin, Paclitaxel, Paclitaxel Albumin-stabilized Nanoparticle Formulation, PAD, Padcev, Palbociclib, Palifermin, Palonosetron Hydrochloride, Pamidronate Disodium, Panitumumab, Paraplatin, Pazopanib Hydrochloride, PCV, PEB, Pegaspargase, Pegfilgrastim, Peginterferon Alfa-2b, PEG-Intron, Pemazyre, Pembrolizumab, Pemetrexed Disodium, Pemigatinib, Perjeta, Pertuzumab, Pexidartinib Hydrochloride, Phesgo, Piqray, Plerixafor, Polatuzumab Vedotin-piiq, Polivy, Pomalidomide, Pomalyst, Ponatinib Hydrochloride, Portrazza, Poteligeo, Pralatrexate, Pralsetinib, Prednisone, Procarbazine Hydrochloride, Procrit, Proleukin, Prolia, Promacta, Propranolol Hydrochloride, Provenge, Purinethol, Purixan, Qinlock, Radium 223 Dichloride, Raloxifene Hydrochloride, Ramucirumab, Rasburicase, Ravulizumab-cwvz, Reblozyl, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor, Relugolix, R-EPOCH, Retacrit, Retevmo, Revlimid, Ribociclib, R-ICE, Ripretinib, Rituxan, Rituxan Hycela, Rituximab, Rolapitant Hydrochloride, Romidepsin, Romiplostim, Ropeginterferon Alfa-2b-njft, Rozlytrek, Rubidomycin, Rubraca, Rucaparib Camsylate, Ruxolitinib Phosphate, Rybrevant, Rydapt, Rylaze, Sacituzumab Govitecan-hziy, Sancuso, Sarclisa, Sclerosol Intrapleural Aerosol, Selinexor, Selpercatinib, Selumetinib Sulfate, Scemblix, Siltuximab, Sipuleucel-T, Sirolimus Protein-Bound Particles, Soltamox, Somatuline Depot, Sonidegib, Sorafenib Tosylate, Sotorasib, Sprycel, STANFORD V, Sterile Talc Powder, Steritalc, Stivarga, Sunitinib Malate, Sustol, Sutent, Sylatron, Sylvant, Synribo, Tabloid, Tabrecta, TAC, Tafasitamab-cxix, Tafinlar, Tagraxofusp-erzs, Tagrisso, Talazoparib Tosylate, Talimogene Laherparepvec, Talzenna, Tamoxifen Citrate, Tarceva, Targretin, Tasigna, Tavalisse, Taxotere, Tazemetostat Hydrobromide, Tazverik, Tebentafusp-tebn, Tecartus, Tecentriq, Temodar, Temozolomide, Temsirolimus, Tepadina, Tepmetko, Tepotinib Hydrochloride, Thalidomide, Thalomid, Thioguanine, Thiotepa, Tibsovo, Tisagenlecleucel, Tisotumab Vedotin-tftv, Tivdak, Tivozanib Hydrochloride, Tocilizumab, Tolak, Topotecan Hydrochloride, Toremifene, Torisel, Totect, TPF, Trabectedin, Trametinib Dimethyl Sulfoxide, Trastuzumab, Trastuzumab and Hyaluronidase-oysk, Treanda, Trexall, Trifluridine and Tipiracil Hydrochloride, Trisenox, Trodelvy, Truseltiq, Truxima, Tucatinib, Tukysa, Turalio, Tykerb, Ukoniq, Ultomiris, Umbralisib Tosylate, Undencyca, Unituxin, Uridine Triacetate, VAC, Valrubicin, Valstar, Vandetanib, VAMP, Varubi, Vectibix, VelP, Velcade, Vemurafenib, Venclexta, Venetoclax, Verzenio, Vidaza, Vinblastine Sulfate, Vincristine Sulfate, Vincristine Sulfate Liposome, Vinorelbine Tartrate, VIP, Vismodegib, Vistogard, Vitrakvi, Vizimpro, Voraxaze, Vorinostat, Votrient, Vyxeos, Welireg, Xalkori, Xatmep, Xeloda, XELIRI, XELOX, Xgeva, Xofigo, Xospata, Xpovio, Xtandi, Yervoy, Yescarta, Yondelis, Yonsa, Zaltrap, Zanubrutinib, Zarxio, Zejula, Zelboraf, Zepzelca, Zevalin, Ziextenzo, Zinecard, Zirabev (Bevcizumab), Ziv-Aflibercept, Zofran, Zoladex, Zoledronic Acid, Zolinza, Zometa, Zyclara, Zydelig, Zykadia, Zynlonta, and Zytiga.

In embodiments, the payload is one or more of pidilizumab, BMS-936559, tremelimumab, AGEN1884, and/or RG2077.

In embodiments, the nucleic acid molecule encodes one or more of a CRISPR/Cas component, guide RNA (gRNA), tracer RNA (tracrRNA), micro RNA (miRNA), RNA inference (RNAi), small interference RNA (siRNA), duplex RNA, Piwi-interacting RNA (piRNA), small nuclear RNA (snRNA), small nucleolar RNA (snoRNA), antisense oligonucleotide (ASO), locked nucleic acid (LNA), splice switching oligonucleotide (SSO), tRNA, ribosomal RNA (rRNA), short hairpin (shRNA), complementary messenger RNA, repeat associated small interfering RNA (rasiRNA), and/or small non-coding RNA.

In embodiments, the gene editing payload includes gene editing nucleic acids and/or proteins, such as for example, TALENs, ZFNs, RNase P RNA, C2c1, C2c2, C2c3, Cas9, Cpf1, TevCas9, Archaea Cas9, CasY.1, CasY.2, CasY.3, CasY.4, CasY.5, CasY.6, CasX Cas omega, transposase, and/or any ortholog or homolog of any of these editors. In embodiments, the gene editors can also include gRNA, which, as used herein, refers to guide RNA. In embodiments, the gRNA can be a sequence complimentary to a coding or a non-coding sequence and can be tailored to the particular sequence to be targeted. In embodiments, the gRNA sequence can be a sense or anti-sense sequence. In embodiments, when a gene editor composition is administered herein, preferably without limitation, including two or more gRNAs; however, a single gRNA can also be used.

In embodiments, BioNVs deliver a gene editing payload comprising a transactivating response region (TAR) loop system. In embodiments, the BioNV encapsulates a plasmid which expresses a gene editor and contains a TAR loop sequence between the 5′ end of the promoter and the gene editor/guide cassette and acts as a barrier, blocking transcription. In embodiments, transcription will only trigger in cells that are infected and contain the HIV Tat protein. In embodiments, the Tat protein binds to the TAR Loop, relaxes it, and frees the promoter for transcription, thereby expressing the editor and its guides.

In embodiments, the BioNV encapsulates one or more perforin molecules. In embodiments, the BioNV encapsulates one or more granzyme molecules. In embodiments, the granzyme molecules are selected from granzyme A, B, H, K, and M. In embodiments, the BioNV encapsulates one or more perforin molecules and/or one or more granzyme molecules derived from a cell from which the BioNV is derived. In embodiments, the BioNV encapsulates one or more perforin molecules and one and/or more granzyme molecules exogenously added to the BioNV. During the cell processing stages (e.g., serial extrusion), in embodiments, the chambers between the extrusion filters can be filled with a concentration gradient of extracellular, purified perforin, granzyme, etc., that can become encapsulated in the BioNVs during the extrusion. In embodiments, BioNVs that express PD-L1 are derived from non-activated cells and the BioNVs substantially lack perforin and/or granzyme.

In embodiments, methods of treating, preventing, and/or ameliorating cancer include co-administering a whole cell therapy (e.g., T cell, NK cell, TIL, macrophage therapy). In embodiments, supplementing a whole cell therapy with BioNVs can be used to decrease the effective dosage of the whole cell therapy needed, reducing CRS, teratoma potential, off-target effects, and the like.

In embodiments, methods of treating or preventing cancer herein include administering an additional therapeutic agent. In embodiments, the additional therapeutic agent can be any additional anti-cancer agent, analgesic, and/or non-steroidal inflammatory agent (NSAID).

In embodiments, BioNVs can be frozen at −80° C., or is suitable for storage at about −80° C., and/or lyophilized (e.g., for reconstitution in buffer). In embodiments, BioNVs can be stable at about ambient temperature, at about-20° C., at about 4° C., at about 25° C., or at about 37° C. for at least about 1 hour, at least about 2 hours, at least about 4 hours, at least about 6 hours, at least about 12 hours, at least about 24 hours, at least about 2 days, at least about one week, or at least about one month or longer.

In embodiments, the PD-1 inhibiting agent is an antibody targeted against PD-1, or pembrolizumab, nivolumab, or cemiplimab. In embodiments, the PD-L1 or PD-L2 inhibiting agent is an antibody targeted against PD-L1 or PD-L2, optionally atezolizumab, avelumab, or durvalumab, or the antigen-binding domain thereof, and is conjugated to the surface of the BioNV. In embodiments, the PD-1 inhibiting agent is an antibody targeted against PD-1, or pembrolizumab, nivolumab, or cemiplimab. In embodiments, the PD-L1 or PD-L2 inhibiting agent is an antibody targeted against PD-L1 or PD-L2, optionally atezolizumab, avelumab, or durvalumab, or the antigen-binding domain thereof, and is membrane-anchored via a transmembrane domain fusion. In embodiments, antibodies can be membrane-anchored via any transmembrane domain that is suitable for a CAR. In embodiments, “conjugated” can refer to chemical association of the antibody to the lipid layer of the BioNV, for example via covalent conjugation via a linker; linkers for antibodies and antibody-drug conjugates are well known in the art.

In embodiments, methods of treatment include BioNVs with an integrated antibody or antibody format (e.g., pembrolizumab or nivolumab). In embodiments, “integrated” can refer to integrating the antibody into the lipid layer of the BioNV. In embodiments, the antibody can be expressed on the surface of the modified cell from which the BioNVs are derived. In embodiments, the antibodies are engineered to have a transmembrane domain to allow the antibody or antibody format to be surface-exposed on the cell prior to BioNV formation.

In embodiments, the cancer is one or more of a carcinoma, a sarcoma, a myeloma, a leukemia, a lymphoma, a mixed type cancer, and/or a metastatic cancer.

In embodiments, the cancer is selected from Acute Biphenotypic Leukemia, Acute Eosinophilic Leukemia, Acute Lymphoblastic Leukemia, Acute Lymphocytic Leukemia, Acute Myeloid Dendritic Cell Leukemia, Acute Myeloid Leukemia, Adenocarcinoma of the Lung, Adrenal Gland Tumors, Adrenocortical Carcinoma, AIDS-related Cancers, AIDS-related Lymphoma, Alveolar Soft Part and Cardiac Sarcoma, Amyloidosis, Anal Cancer, Anaplastic Large Cell Lymphoma, Angioimmunoblastic T-cell Lymphoma, Appendix Cancer, Astrocytoma, Ataxia-telangiectasia, Attenuated Familial Adenomatous Polyposis, B-cell Prolymphocytic Leukemia, Basal Cell Carcinoma, Beckwith-Wiedemann Syndrome, Bile Duct Cancer, Birt-Hogg-Dubé Syndrome, Bladder Cancer, Bone Cancer, Brain and Nervous System Cancer, Brain Stem Glioma, Brainstem Glioma, Brain Tumors, Breast Cancer, Bronchial Adenomas/Carcinoids, Burkitt's Lymphoma, Carcinoid Tumor, Carcinoid Tumors, Carney Complex, Central Nervous System Tumors, Cerebellar Astrocytoma, Cerebral Astrocytoma, Cervical Cancer, Childhood Desmoplastic Ganglioglioma, Cholangiocarcinoma, Chondrosarcoma, Chronic Lymphocytic Leukemia, Chronic Myelogenous Leukemia, Chronic Myeloid Leukemia, Chronic T-cell Lymphocytic Leukemia, Colon Cancer, Colorectal Cancer, Cowden Syndrome, Craniopharyngioma, Cutaneous T-cell Lymphoma, Dermatofibrosarcoma Protuberans, Desmoplastic Small Round Cell Tumor, Diffuse Gastric Cancer, Diffuse Large B-cell Lymphoma, Endocrine System Cancer, Endocrine Tumors, Endometrial Cancer, Eosinophilic Leukemia, Ependymoma, Epithelioid Hemangioendothelioma (EHE), Esophageal Cancer, Ewing Sarcoma, Extragonadal Germ Cell Tumor, Extrahepatic Bile Duct Cancer, Eye Cancer, Eyelid Cancer, Fallopian Tube Cancer, Familial Adenomatous Polyposis, Familial Malignant Melanoma, Familial Clear Cell Renal Cell Carcinoma (RCC), Follicular Lymphoma, Gallbladder Cancer, Gardner Syndrome, Gastric (stomach) Cancer, Gastrointestinal Cancer, Gastrointestinal Carcinoid Tumor, Gastrointestinal Stromal Tumor, Gastrointestinal Stromal Tumor (GIST), Genitourinary and Gynecologic Cancer, Germ Cell Tumor, Gestational Trophoblastic Disease, Gestational Trophoblastic Tumor, Glioblastoma, Glioma, Hairy Cell Leukemia, Head and Neck Cancer, Hematopoietic Cancer, Hepatocellular Cancer, Hepatosplenic T-cell Lymphoma, HIV-related Cancer, Hodgkin's Lymphoma, Hypopharyngeal Cancer, Inflammatory Breast Cancer, Intravascular Large B-cell Lymphoma, Invasive Cribriform Carcinoma, Invasive Lobular Carcinoma, Islet Cell Carcinoma (endocrine Pancreas), Islet Cell Tumors, Juvenile Polyposis Syndrome, Kaposi Sarcoma, Keratoacanthoma, Kidney Cancer, Lacrimal Gland Tumor, Large Granular Lymphocytic Leukemia, Laryngeal and Hypopharyngeal Cancer, Leiomyomatosis and Renal Cell Cancer, Leiomyosarcoma, Li-Fraumeni Syndrome (LFS), Liposarcoma, Liver Cancer, Lung Cancer, Lymphomas of Primary Cutaneous Origin, Lymphomatoid Granulomatosis, Lymphoplasmacytic Lymphoma, Lynch Syndrome, Malignant Fibrous Histiocytoma of Bone, Mantle Cell Lymphoma, Marginal Zone B-cell Lymphoma, Mast Cell Leukemia, Mastocytosis, Mediastinal Large B Cell Lymphoma, Medullary Carcinoma, Medulloblastoma, Melanoma, Meningioma, Merkel Cell Carcinoma, Mesothelioma, Mixed Polyposis Syndrome, Mucosa-associated Lymphoid Tissue Lymphoma, Muir-Torre Syndrome (MTS), Multiple Endocrine Neoplasia Syndrome, Multiple Endocrine Neoplasia Type 1, Multiple Endocrine Neoplasia Type 2, Multiple Myeloma, Multiple Myeloma/Plasma Cell Neoplasm, Mycosis Fungoides, Myelodysplastic Syndromes, MYH-associated Polyposis, Myxosarcoma, Nasal Cavity and Paranasal Sinus Cancer, Nephroblastoma, Nasopharyngeal Cancer, Nasopharyngeal Carcinoma, Neuroblastoma, Neuroendocrine Tumors, Neurofibromatosis Type 1, Neurofibromatosis Type 2, Nevoid Basal Cell Carcinoma Syndrome, Nodal Marginal Zone B Cell Lymphoma, Non-Hodgkin Lymphoma, Non-small Cell Lung Cancer, Non-small Cell Lung Carcinoma, Oligodendroglioma, Optic Nerve Glioma, Oral and/or Oropharyngeal Cancer, Osteosarcoma, Ovarian Cancer, Ovarian Epithelial Cancer, Ovarian Germ Cell Tumor, Pancreatic Cancer, Papillary Renal Cell Carcinoma, Paranasal Sinus and Nasal Cavity Cancer, Parathyroid Cancer, Pelvic Cancer, Penile Cancer, Peutz-Jeghers Syndrome, Pharyngeal Cancer, Pheochromocytoma, Phyllodes Tumor, Pilocytic Astrocytoma, Pineal Astrocytoma, Pituitary Adenoma, Pituitary Gland Tumors, Plasmablastic Lymphoma, Pleuropulmonary Blastoma, Precursor B Lymphoblastic Leukemia, Primary Central Nervous System Lymphoma, Primary Cutaneous Follicular Lymphoma, Primary Cutaneous Immunocytoma, Primary Effusion Lymphoma, Primitive Neuroectodermal Tumor, Prostate Cancer, Rectal Cancer, Renal Cancer, Renal Cell Carcinoma, Retinoblastoma, Rhabdomyosarcoma, Salivary Gland Cancer, Sarcoma, Sarcomas of Primary Cutaneous Origin, Sebaceous Carcinoma, Sezary Syndrome, Skin Adnexal Tumors, Skin Cancer, Small Bowel Cancer, Small Cell Lung Cancer, Small Intestine Cancer, Splenic Marginal Zone Lymphoma, Squamous-cell Carcinoma of the Lung, Squamous Cell Carcinoma, Squamous Cell Skin Cancer, Stomach Cancer, Surface Epithelial-Stromal Tumor, T-cell Prolymphocytic Leukemia, Testicular Cancer, Thoracic and Respiratory Cancer, Thymoma, Thymic Carcinoma, Thyroid Cancer, Transitional Cell Cancer, Transitional Cell Canceradenoid Cystic Carcinoma, Tuberous Sclerosis Syndrome, Tubular Carcinoma, Turcot Syndrome, Unknown Primary Cancer, Unsorted Cancer, Ureter Cancer, Urethral Cancer, Uterine Cancer, Uterine Sarcoma, Uveal Melanoma, Vaginal Cancer, Visual Pathway and Hypothalamic Glioma, Von Hippel-Lindau (VHL) Syndrome, Vulvar Cancer, Wilms Tumor, and Xeroderma Pigmentosum.

In embodiments, the BioNVs can target cancer cells associated with adenoid cystic carcinoma, adrenal gland tumors, amyloidosis, anal cancer, appendix cancer, astrocytoma, ataxia-telangiectasia, attenuated familial adenomatous polyposis, Beckwith-Wiedemann Syndrome, bile duct cancer, Birt-Hogg-Dube Syndrome, bladder cancer, bone cancer, brain stem glioma, brain tumors, breast cancer, carcinoid tumors, Carney complex, central nervous system tumors, cervical cancer, colorectal cancer, Cowden syndrome, craniopharyngioma, desmoplastic infantile ganglioglioma, endocrine tumors, ependymoma, esophageal cancer, Ewing sarcoma, eye cancer, eyelid cancer, fallopian tube cancer, familial adenomatous polyposis, familial malignant melanoma, familial non-VHL clear cell renal cell carcinoma, gallbladder cancer, Gardner Syndrome, gastrointestinal stromal tumor, germ cell tumor, gestational trophoblastic disease, head and neck cancer, diffuse gastric cancer, leiomyomatosis and renal cell cancer, mixed polyposis syndrome, pancreatitis, papillary renal cell carcinoma, HIV and AIDS-related cancer, islet cell tumors, juvenile polyposis syndrome, kidney cancer, lacrimal gland tumor, laryngeal and hypopharyngeal cancer, acute lymphoblastic leukemia, acute lymphocytic leukemia, acute myeloid leukemia, B-cell prolymphocytic leukemia, hairy cell leukemia, chronic lymphocytic leukemia, chronic myeloid leukemia, chronic T-cell lymphocytic leukemia, eosinophilic leukemia, Li-Fraumeni Syndrome, liver cancer, lung cancer, Hodgkin lymphoma, Non-Hodgkin lymphoma, Lynch Syndrome, mastocytosis, medulloblastoma, melanoma, meningioma, mesothelioma, Muir-Torre Syndrome, multiple endocrine neoplasia type 1, multiple endocrine neoplasia type 2, multiple myeloma, myelodysplastic syndromes, MYH-associated polyposis, nasal cavity and paranasal sinus cancer, nasopharyngeal cancer, neuroblastoma, neuroendocrine tumors, neurofibromatosis type 1, neurofibromatosis type 2, nevoid basal cell carcinoma syndrome, oral and oropharyngeal cancer, osteosarcoma, ovarian cancer, pancreatic cancer, parathyroid cancer, penile cancer, Peutz-Jeghers Syndrome, pituitary gland tumors, pleuropulmonary blastoma, prostate cancer, retinoblastoma, rhabdomyosarcoma, salivary gland cancer, sarcoma, alveolar soft part and cardiac sarcoma, Kaposi sarcoma, skin cancer, small bowel cancer, stomach cancer, testicular cancer, thymoma, thyroid cancer, tuberous sclerosis syndrome, Turcot Syndrome, unknown primary, uterine cancer, vaginal cancer, Von Hippel-Lindau Syndrome, Wilms tumors, or Xeroderma pigmentosum.

In embodiments, treating, preventing, and/or ameliorating a cancer includes one or more of clinical remission, reduction in tumor volume, diminished angiogenesis, reduced size and number of metastasis, increased tumor infiltration, reduced tumor volume, or amelioration of a cancer-associated clinical symptomology, within about 2 weeks, within about 4 weeks, within about 6 weeks, within about 12 weeks, within about 18 weeks, within about 24 weeks, within about 6 months, within about 1 year, or within about 2 or more years from administration of the composition and methods with such compositions.

### Dosing and Administration

The dosage of any BioNVs disclosed herein as well as the dosing schedule can depend on various parameters and factors, including, but not limited to, the specific BioNVs, the cancer being treated, the severity of the condition, whether the condition is to be treated or prevented, the subject's age, weight, and general health, and the administering physician's discretion. Additionally, pharmacogenomic (the effect of genotype on the pharmacokinetic, pharmacodynamic or efficacy profile of a therapeutic) information about a particular subject may affect dosage used. Furthermore, the exact individual dosages can be adjusted somewhat depending on a variety of factors, including the specific combination of the agents being administered, the time of administration, the route of administration, the nature of the formulation, the rate of excretion, the disease being treated, the severity of the disorder, and the anatomical location of the disorder. Some variations in the dosage can be expected.

In embodiments, delivery of BioNVs can be like that of a vesicle, in particular a liposome (see Langer, 1990, Science 249:1527-1533; Treat et al., in Liposomes in Therapy of Infectious Disease and Cancer, Lopez-Berestein and Fidler (eds.), Liss, New York, pp. 353-365 (1989).

Methods of treating and/or preventing cancer using BioNVs described herein, in embodiments, include dosage ranges in concentration of number of BioNVs per kilogram (kg) subject body weight. In embodiments, suitable dosage ranges for methods described herein can include from about 10³ BioNVs/kg to about 10¹² BioNVs/kg. In embodiments, the BioNVs are present in the composition at a concentration of about 10³ BioNVs/mL to about 10¹⁴ BioNVs/mL. Alternatively, in embodiments, BioNV compositions are present in compositions as weight/volume in the range of about 5 ng/ml to about 500 mg/mL. In embodiments, the BioNV dosages are based on the size of the BioNVs used for the treatment, for example, BioNVs at 1000 nm are provided in approximately 5-fold to 10-fold fewer amounts than 100 nm BioNVs for a comparable dose.

In embodiments, BioNVs disclosed herein are administered by a controlled-release or a sustained-release means or by delivery of a device that is well known to those of ordinary skill in the art. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,556, each of which is incorporated herein by reference in its entirety. Such dosage forms can be useful for providing controlled or sustained-release of one or more active ingredients using, for example, hydropropylmethyl cellulose, other polymer matrices, gels, permeable membranes, osmotic systems, multilayer coatings, microparticles, microspheres, or a combination thereof, to provide the desired release profile in varying proportions. Controlled- or sustained-release of an active ingredient can be stimulated by various conditions, including but not limited to, changes in pH, changes in temperature, stimulation by an appropriate wavelength of light, concentration or availability of enzymes, concentration or availability of water, or other physiological conditions or compounds.

In embodiments, polymeric materials are used (see Medical Applications of Controlled Release, Langer and Wise (eds.), CRC Pres., Boca Raton, Florida (1974); Controlled Drug Bioavailability, Drug Product Design and Performance, Smolen and Ball (eds.), Wiley, New York (1984); Ranger and Peppas, 1983, J. Macromol. Sci. Rev. Macromol. Chem. 23:61; Levy et al., 1985, Science 228:190; During et al., 1989, Ann. Neurol. 25:351; Howard et al., 1989, J. Neurosurg. 71:105).

In embodiments, a controlled-release system is placed in proximity of the target area to be treated, thus requiring only a fraction of the systemic dose (see, e.g., Goodson, in Medical Applications of Controlled Release, supra, vol. 2, pp. 115-138 (1984). Other controlled-release systems discussed in the review by Langer, 1990, Science 249:1527-1533 may be used.

In embodiments, the methods using BioNVs include applying BioNVs to a surface of a device (e.g., a catheter) or contained within a pump, patch, or other drug delivery device. The excipient or carrier can be selected based on the mode and route of administration. Suitable pharmaceutical carriers, as well as pharmaceutical necessities for use in pharmaceutical formulations, are described in Remington's Pharmaceutical Sciences (E. W. Martin), a well-known reference text in this field, and in the USP/NF (United States Pharmacopeia and the National Formulary).

Pembrolizumab is a humanized anti-PD-1 monoclonal antibody that is used in treating cancers such as melanoma, lung cancer, head and neck cancer, Hodgkin lymphoma, stomach cancer, cervical cancer, and breast cancer. It binds PD-1 on the cancer cell, blocking the interaction with the receptors of the lymphocytes. In embodiments, this allows the immune system to target the cancer cells which can no longer evade the cytotoxic response, while preventing the immune system from attacking healthy cells. In embodiments, pembrolizumab can be administered at doses of 200 mg to 400 mg or 2 mg/kg.

Nivolumab is a humanized anti-PD-1 monoclonal antibody that is used in treating cancers such as melanoma, lung cancer, malignant pleural mesothelioma, renal cell carcinoma, colon cancer, esophageal squamous cell carcinoma, liver cancer, gastric cancer, and esophageal or gastroesophageal junction cancer. In embodiments, nivolumab works in the same manner as pembrolizumab. In embodiments, nivolumab can be administered at doses of 240 mg to 480 mg or 3 mg/kg.

In embodiments, BioNVs can be administered at dosages that are congruent to dosages of pembrolizumab and/or nivolumab. In embodiments, BioNVs can be administered at doses that are congruent to dosages of whole cells, for example, based on CAR concentration. In embodiments, the typical concentration range of CAR protein per microgram of T cells is between 0.20 ng-0.70 ng, whereas a single BioNV may have a total number of CARs that is 5 times to 10,000 times less than the whole cell, resulting in a conversion of BioNV mass to CAR concentration, where the CAR concentration can be assumed equivalent (such as the case in exosomes) or increased (such as the case in BioNVs) to the cell from which it originated (e.g., the T cell). In embodiments, the concentration and/or surface density of the targeting agent (e.g., CAR) is increased on the BioNV compared to the whole cell from which is it derived. In embodiments, the concentration and/or surface density of the targeting agent (e.g., CAR) is enriched by serial extrusion processing of the whole cell. In embodiments, the concentration and/or surface density of the targeting agent (e.g., CAR), among other cell surface molecules, on the BioNV is 2-fold to 100-fold increased relative to the whole cell.

The dosage regimen utilizing any BioNVs disclosed herein can be selected in accordance with a variety of factors including cancer type, species, age, weight, sex, and medical condition of the subject; the severity of the condition to be treated; the route of administration; the renal or hepatic function of the subject; the pharmacogenomic makeup of the individual; and the specific composition of the disclosure employed. Any BioNVs disclosed herein can be administered in a single daily dose, or the total daily dosage can be administered in divided doses of two, three, or four times daily. Furthermore, any BioNVs disclosed herein can be administered continuously rather than intermittently throughout the dosage regimen.

In embodiments, BioNVs are administered in consecutive doses about every hour, about every 2 hours, about every 6 hours, about every 12 hours, about every 24 hours, about every 2 days, about every 4 days, about every 7 days, about every 2 weeks, about every 4 weeks, or about every month.

In embodiments, a combined remission or clinical remission of the cancer, reduction in tumor volume, diminished angiogenesis, reduced size and number of metastasis, increased tumor infiltration, reduced primary tumor volume, or amelioration of clinical symptomology, is achieved within about 24 weeks, about 18 weeks, about 12 weeks, about 8 weeks, about 6 weeks, about 4 weeks, about 2 weeks, or about 1 week from administration of the composition and methods with such compositions.

### Compositions of BioNVs

In aspects, the present disclosure relates to compositions for the treatment and/or prevention of cancer comprising an allogeneic BioNV comprising a membrane-embedded CAR targeted against a cell surface marker, and a conjugated and/or membrane-anchored targeting agent targeted against PD-L1, or PD-L2, wherein the agent targeted against PD-L1 or PD-L2 is atezolizumab, avelumab, or durvalumab, and wherein the CAR is not targeted against PD-1, PD-L1, or PD-L2.

In aspects, the present disclosure relates to compositions for the treatment and/or prevention of cancer comprising a first membrane-embedded CAR targeted against a cell surface, and at least a second membrane-embedded CAR targeted against PD-L1 or PD-L2, wherein the first CAR is not targeted against PD-1, PD-L1, or PD-L2.

In aspects, the present disclosure relates to compositions for the treatment and/or prevention of cancer comprising a bispecific, membrane-embedded CAR targeted against a cell surface marker of a cancer cell, and PD-L1 or PD-L2, wherein the cell surface marker of the cancer cell is not PD-1, PD-L1, or PD-L2.

In embodiments, compositions include BioNVs. In embodiments, compositions include a BioNV and at least an anti-cancer therapeutic, as described herein. In embodiments, the BioNVs can adsorb therapeutic molecules onto the surface of the NV and/or encapsulate a therapeutic payload within an aqueous compartment of the NV. In embodiments, compositions include a BioNV and at least one checkpoint inhibitor encapsulated within the aqueous core. In embodiments, the composition comprises a therapeutically effective amount of the BioNVs. In embodiments, the BioNV and checkpoint inhibitor(s) can be combined in solution or can be in separate solutions to be co-administered. In embodiments, the composition comprises BioNVs loaded with cytotoxic molecules, such as perforin, granzyme, etc.

In embodiments, the composition is derived from iPSCs (among other cell types) which have been modified to reduce expression of immunogenic molecules and/or increase expression of immunoprotective molecules.

In embodiments, the composition is allogenic and/or hypoimmunogenic. For example, in embodiments, the composition does not result in an inflammatory reaction and/or an immune response upon administration. In embodiments, the BioNVs are allogeneic and/or hypoimmunogenic. In embodiments, upon administration to a subject, the composition, optionally the BioNVs therein, elicits less than about 50%, about 45%, about 40%, about 35%, about 30%, about 25%, about 24%, about 23%, about 22%, about 21%, about 20%, about 19%, about 18%, about 17%, about 16%, about 15%, about 14%, about 13%, about 12%, about 11%, about 10%, about 9%, about 8, about 7%, about 6%, about 5%, about 4%, about 3%, about 2%, or about 1% of an inflammatory or immune response measured as a function of cytokine, chemokine, or immunomodulatory enzyme concentration, such as IL-1, IL-2, IL-3, IL-4, IL-6, IL-7, IL-9, IL-10, IL-11, IL-12, IL-13, IL-14, IL-15, IL-16, IL-17, IL-20, IFN-α/β/γ, TNFα/β, IDO, HLA-G, HGF, PGE2, among others, or any combination thereof, in comparison, e.g. to a cognate whole cell therapy counterpart.

In embodiments, the BioNVs are present in the composition at a concentration of about 10³ BioNVs/mL to about 10¹⁴ BioNVs/mL. Alternatively, in embodiments, BioNV compositions are present in compositions as weight/volume in the range of about 5 ng/ml to about 500 mg/mL.

In embodiments, the composition is substantially free of one or more bacteria, virus, fungus, spore, mycoplasma, pyrogen, and in more particular embodiments, is substantially free of all the foregoing. In embodiments, the composition is substantially free of whole cells and intracellular cell components including organelles such as nuclei, mitochondria, Golgi, etc., and/or substantially free of non-CAR-expressing NVs and/or substantially free of ruptured, damaged NVs. In embodiments, the composition is substantially free of cellular chromatin, nucleosomes, genomic DNA, among other cellular genetic material and non-therapeutic nucleic acids.

### Pharmaceutical Compositions and Formulations of BioNVs

In aspects, the composition is a pharmaceutical composition. In embodiments, the pharmaceutical compositions of the present disclosure are formulated to provide a therapeutically effective amount of BioNVs as the active ingredient. In embodiments, the pharmaceutical compositions of the present disclosure are formulated to provide a therapeutically effective amount of one or more anticancer therapeutics as a payload within a BioNV as the active ingredient. Typically, the pharmaceutical compositions also comprise one or more pharmaceutically acceptable excipients, carriers, including inert solid diluents and fillers, diluents, including sterile aqueous solution and various organic solvents, permeation enhancers, solubilizers and adjuvants.

Pharmaceutical excipients can be liquids, such as water and oils, including those of petroleum, animal, vegetable, or synthetic origin, such as peanut oil, soybean oil, mineral oil, sesame oil and the like. The pharmaceutical excipients can be, for example, saline, gum acacia, gelatin, starch paste, talc, keratin, colloidal silica, urea, and the like. In addition, auxiliary, stabilizing, thickening, lubricating, and coloring agents can be used. Pharmaceutically acceptable excipients are generally sterile when administered to a subject. Water is a useful excipient when any agent disclosed herein is administered intravenously. Saline solutions and aqueous dextrose and glycerol solutions can also be employed as liquid excipients, specifically for injectable solutions. Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. Any composition disclosed herein, if desired, can also formulated with wetting or emulsifying agents, or pH buffering agents. Other examples of suitable pharmaceutical excipients are described in Remington's Pharmaceutical Sciences 1447-1676 (Alfonso R. Gennaro eds., 19th ed. 1995), incorporated herein by reference.

In embodiments, the composition comprises an excipient or carrier. In embodiments, the diluent is a pharmaceutically acceptable excipient or carrier.

In embodiments, the pharmaceutical composition comprises a pharmaceutically acceptable diluent. Non-limiting example of diluents include liquid diluents such as water, ethanol, propylene glycol, glycerin, and various combinations thereof, and inert solid diluents such as calcium carbonate, calcium phosphate or kaolin. In embodiments, the diluent comprises one or more of saline, phosphate buffered saline, Dulbecco's Modified Eagle Medium (DMEM), alpha modified Minimal Essential Medium (alpha MEM), Roswell Park Memorial Institute Media 1640 (RPMI Media 1640), HBSS, human albumin, Ringer's solution, and the like, or any combination thereof.

In embodiments, the active ingredient is typically mixed with an excipient, diluted by an excipient or enclosed within such a carrier in the form of, for example, a capsule, tablet, sachet, paper, or other container. When the excipient serves as a diluent, it can be a solid, semisolid, or liquid material (e.g., normal saline), which acts as a vehicle, carrier or medium for the active ingredient. In embodiments, the compositions can be in the form of tablets, pills, powders, lozenges, sachets, cachets, elixirs, suspensions, emulsions, solutions, syrups, aerosols (as a solid or in a liquid medium), lotions, creams, ointments, gels, soft and hard gelatin capsules, suppositories, sterile injectable solutions, and sterile packaged powders. As is known in the art, the type of diluent can vary depending upon the intended route of administration. In embodiments, the resulting compositions can include additional agents, such as preservatives, cryopreservatives (e.g., DMSO), and/or lyoprotectants (e.g., polyols, salts). In embodiments, the carrier can be, or can include a lipid-based or polymer-based colloid. In embodiments, the carrier material can be a colloid formulated as a liposome, a hydrogel, a microparticle, a nanoparticle, or a block copolymer micelle. In embodiments, the carrier material can form a capsule, and that material may be a polymer-based colloid.

In embodiments, the pharmaceutical compositions comprising the BioNVs include a solubilizing agent. In embodiments, the pharmaceutical compositions comprising the BioNVs include a cryoprotective agent or an agent to improve thermal stability, such as DMSO or glycerol. The pharmaceutical compositions, in embodiments, can be delivered with a suitable vehicle or delivery device as known in the art.

In embodiments, the composition comprises a scaffold. In embodiments, the scaffold comprises biomaterials. In a non-limiting example, the three-dimensional biomaterials include BioNVs embedded in an extracellular matrix attached to, or dispersed within, or trapped within the scaffold. In embodiments, the biomaterials are biodegradable and/or synthetic.

In embodiments, the scaffold comprises biodegradable biomaterials. Non-limiting examples of biodegradable biomaterials include fibrin, collagen, elastin, gelatin, vitronectin, fibronectin, laminin, reconstituted basement membrane matrix, starch, dextran, alginate, hyaluron, chitin, chitosan, agarose, sugars, hyaluronic acid, poly (lactic acid), poly (glycolic acid), polyethylene glycol, decellularized tissue, self-assembling peptides, polypeptides, glycosaminoglycans, derivatives and mixtures thereof. Other useful biodegradable polymers or polymer species include, but are not limited to, polydioxanone, polycarbonate, polyoxalate, poly (α-ester), polyanhydride, polyacetate, polycaprolactone, poly (ortho Esters), polyamino acids, polyamides, and mixtures and copolymers thereof, L-lactic acid and D-lactic acid stereopolymers, copolymers of bis (para-carboxyphenoxy) propanoic acid and sebacic acid, sebacic acid copolymers, caprolactone Copolymer, poly (lactic acid)/poly (glycolic acid)/polyethylene glycol copolymer, polyurethane and poly (lactic acid) copolymer, polyurethane and poly (lactic acid) copolymer, α-amino acid copolymer, α-amino acid and caproic acid copolymer, A-benzylglutamate and polyethylene glycol copolymers, succinate and poly (glycol) copolymers, polyphosphazenes, polyhydroxy-alkanoates and mixtures thereof. Binary and ternary systems are also contemplated. In embodiments, the scaffold comprises one or more of collagen, various proteoglycans, alginate-based substrates, and chitosan. In embodiments, the scaffold comprises one or more of a hydrogel, silk, Matrigel, acellular and/or decellarized scaffolds, poly-ε-caprolactone scaffolds, resorbable scaffolds, and nanofiber-hydrogel composite.

In embodiments, the scaffold comprises synthetic biomaterials. Non-limiting examples of synthetic biomaterials include lactone-based polyesters or copolyesters such as polylactide, polycaprolactonglycolide, polyorthoesters, polyanhydrides, polyaminoacids, polysaccharides, polyphosphazenes, poly (ether-ester) copolymers (e.g., PEO-PLLA); polydimethylsiloxane, poly (ethylene-vinylacetate), acrylate-based polymers or copolymers (e.g., polyhydroxyethyl methylmethacrylate, polyvinyl pyrrolidinone), fluorinated polymers such as polytetrafluoroethylene and cellulose esters.

In embodiments, the compositions can be prepared in any manner well known in the pharmaceutical arts, and can be administered by a variety of routes (e.g., subcutaneous, intravenous, etc.) depending upon whether local or systemic treatment is desired and upon the area to be treated. In embodiments, administration can be topical (including ophthalmic and to mucous membranes including intranasal, vaginal, and rectal delivery), pulmonary (e.g., by inhalation or insufflation of powders or aerosols, including by nebulizer; intratracheal, intranasal, epidermal and transdermal), ocular, oral, or parenteral. In embodiments, methods can include ocular delivery, topical administration (eye drops), subconjunctival, periocular or intravitreal injection or introduction by balloon catheter or ophthalmic inserts surgically placed in the conjunctival sac. In embodiments, parenteral administration includes intravenous, intra-arterial, subcutaneous, intraperitoneal or intramuscular injection or infusion; or intracranial, e.g., intrathecal or intraventricular administration. In embodiments, parenteral administration can be in the form of a single bolus dose, or may be, for example, by a continuous perfusion pump.

In embodiments, pharmaceutical compositions and formulations for topical administration may include transdermal patches, ointments, lotions, creams, gels, drops, suppositories, sprays, liquids, powders, and the like. In embodiments, methods of treating and/or preventing cancer include the use of pharmaceutical carriers, aqueous, powder or oily bases, thickeners, and the like.

In embodiments, the pharmaceutical compositions contain, as the active ingredient, nucleic acids and vectors described herein in combination with one or more pharmaceutically acceptable carriers. In embodiments, the terms “pharmaceutically acceptable” (or “pharmacologically acceptable”) refer to molecular entities and compositions that do not produce an adverse, allergic or other untoward reaction, when administered to an animal or a human, as appropriate. The methods and compositions disclosed herein can be applied to a wide range of species, e.g., humans, non-human primates (e.g., monkeys), horses or other livestock, dogs, cats, ferrets or other mammals kept as pets, rats, mice, or other laboratory animals. In embodiments, the term “pharmaceutically acceptable carrier,” includes any and all solvents, dispersion media, coatings, antibacterial, isotonic and absorption delaying agents, buffers, excipients, binders, lubricants, gels, surfactants and the like, that may be used as media for a pharmaceutically acceptable substance.

In embodiments, the compositions can be applied to a surface of a device (e.g., a catheter) or contained within a pump, patch, or other drug delivery device. In embodiments, the compositions can be administered alone, or in a mixture, in the presence of a pharmaceutically acceptable excipient or carrier (e.g., physiological saline). The excipient or carrier is selected based on the mode and route of administration. Suitable pharmaceutical carriers, as well as pharmaceutical necessities for use in pharmaceutical formulations, are described in Remington's Pharmaceutical Sciences (E. W. Martin), a well-known reference text in this field, and in the USP/NF (United States Pharmacopeia and the National Formulary).

In embodiments, the compositions, e.g., pharmaceutical compositions, disclosed herein are suspended in a saline buffer (including, without limitation, TBS, PBS, and the like).

The present technology includes the disclosed BioNVs in various formulations of pharmaceutical compositions. BioNVs disclosed herein, in embodiments, can take the form of solutions, suspensions, emulsion, drops, tablets, pills, pellets, capsules, capsules containing liquids, powders, sustained-release formulations, emulsions, aerosols, sprays, suspensions, or any other form suitable for use.

Pharmaceutical compositions comprising the BioNVs described herein may conveniently be presented in unit dosage forms and may be prepared by any of the methods well known in the art of pharmacy. Such methods generally include the step of bringing therapeutic agents into association with a carrier, which constitutes one or more accessory ingredients. Typically, the pharmaceutical compositions are prepared by uniformly and intimately bringing therapeutic agent into association with a liquid carrier, a finely divided solid carrier, or both, and then, if necessary, shaping the product into dosage forms of the desired formulation (e.g., wet or dry granulation, powder blends, etc., followed by tableting using conventional methods known in the art).

In embodiments, any BioNVs disclosed herein are formulated in accordance with routine procedures as a pharmaceutical composition adapted for a mode of administration disclosed herein.

### Additional Therapeutic Agents

In embodiments, the compositions or methods described herein further comprise a therapeutically effective amount of one or more additional therapeutic agents. In embodiments, the therapeutically effective amount of one or more additional therapeutic agents may be in solution with a BioNV, adsorbed onto the surface of the NV, or a payload encapsulated within a BioNV. In embodiments, the additional therapeutic agent is one or more of a checkpoint inhibitor, an analgesic, and/or an anti-infective agent.

In embodiments, the present compositions or methods contemplate other additional therapeutic agents, for example, an analgesic, to aid in treating inflammation or pain at the site of the administration, or an anti-infective agent to prevent infection of the site of treatment with the composition. Non-limiting examples of additional therapeutic agents include analgesics, such as nonsteroidal anti-inflammatory drugs, opiate agonists and salicylates; anti-infective agents, such as anthelmintics, antianaerobics, antibiotics, aminoglycoside antibiotics, antifungal antibiotics, cephalosporin antibiotics, macrolide antibiotics, miscellaneous B-lactam antibiotics, penicillin antibiotics, quinolone antibiotics, sulfonamide antibiotics, tetracycline antibiotics, antimycobacterials, antituberculosis antimycobacterials, antiprotozoals, antimalarial antiprotozoals, antiviral agents, anti-retroviral agents, scabicides, anti-inflammatory agents, corticosteroid anti-inflammatory agents, antipruritics/local anesthetics, topical anti-infectives, antifungal topical anti-infectives, antiviral topical anti-infectives; electrolytic and renal agents, such as acidifying agents, alkalinizing agents, diuretics, carbonic anhydrase inhibitor diuretics, loop diuretics, osmotic diuretics, potassium-sparing diuretics, thiazide diuretics, electrolyte replacements, and uricosuric agents; enzymes, such as pancreatic enzymes and thrombolytic enzymes; gastrointestinal agents, such as antidiarrheals, antiemetics, gastrointestinal anti-inflammatory agents, salicylate gastrointestinal anti-inflammatory agents, antacid anti-ulcer agents, gastric acid-pump inhibitor anti-ulcer agents, gastric mucosal anti-ulcer agents, H2-blocker anti-ulcer agents, cholelitholytic agents, digestants, emetics, laxatives and stool softeners, and prokinetic agents; general anesthetics, such as inhalation anesthetics, halogenated inhalation anesthetics, intravenous anesthetics, barbiturate intravenous anesthetics, benzodiazepine intravenous anesthetics, and opiate agonist intravenous anesthetics; hormones and hormone modifiers, such as abortifacients, adrenal agents, corticosteroid adrenal agents, androgens, anti-androgens; immunobiological agents, such as immunoglobulins, immunosuppressives, toxoids, and vaccines; local anesthetics, such as amide local anesthetics and ester local anesthetics; musculoskeletal agents, such as anti-gout anti-inflammatory agents, corticosteroid anti-inflammatory agents, gold compound anti-inflammatory agents, immunosuppressive anti-inflammatory agents, nonsteroidal anti-inflammatory drugs (NSAIDs), salicylate anti-inflammatory agents; minerals; vitamins, such as water soluble or fat soluble vitamins, vitamin A, vitamin B, vitamin C, vitamin D, vitamin E, and/or vitamin K; and radionuclides such as Yttrium-90, Iodine-131, Samarium-153, Lutetium-177, Astatine-211, Lead-212/bismuth-212, Radium-223, Actinium-225, and Thorium-227.

Additional non-limiting examples of useful therapeutic agents from the above categories include: (1) analgesics in general, such as lidocaine or derivatives thereof, and NSAID analgesics, including diclofenac, ibuprofen, ketoprofen, and naproxen; (2) opiate agonist analgesics, such as codeine, fentanyl, hydromorphone, and morphine; (3) salicylate analgesics, such as aspirin (ASA) (enteric coated ASA); (4) Hi-blocker antihistamines, such as clemastine and terfenadine; (5) anti-infective agents, such as mupirocin; (6) antianaerobic anti-infectives, such as chloramphenicol and clindamycin; (7) antifungal antibiotic anti-infectives, such as amphotericin b, clotrimazole, fluconazole, and ketoconazole; (8) macrolide antibiotic anti-infectives, such as azithromycin and erythromycin; (9) miscellaneous B-lactam antibiotic anti-infectives, such as aztreonam and imipenem; (10) penicillin antibiotic anti-infectives, such a s nafcillin, oxacillin, penicillin G, and penicillin V; (11) quinolone antibiotic anti-infectives, such as ciprofloxacin and norfloxacin; (12) tetracycline antibiotic anti-infectives, such as doxycycline, minocycline, and tetracycline; (13) antituberculosis antimycobacterial anti-infectives such as isoniazid (INH), and rifampin; (14) antiprotozoal anti-infectives, such as atovaquone and dapsone; (15) antimalarial antiprotozoal anti-infectives, such as chloroquine and pyrimethamine; (16) anti-retroviral anti-infectives, such as ritonavir and zidovudine; (17) antiviral anti-infective agents, such as acyclovir, ganciclovir, interferon alfa, remdesivir, and rimantadine; (18) antifungal topical anti-infectives, such as amphotericin B, clotrimazole, miconazole, and nystatin; (19) antiviral topical anti-infectives, such as acyclovir; (20) electrolytic and renal agents, such as lactulose; (21) loop diuretics, such as furosemide; (22) potassium-sparing diuretics, such as triamterene; (23) thiazide diuretics, such as hydrochlorothiazide (HCTZ); (24) uricosuric agents, such as probenecid; (25) enzymes such as RNase and DNase; (26) antiemetics, such as prochlorperazine; (27) salicylate gastrointestinal anti-inflammatory agents, such as sulfasalazine; (28) gastric acid-pump inhibitor anti-ulcer agents, such as omeprazole; (29) H2-blocker anti-ulcer agents, such as cimetidine, famotidine, nizatidine, and ranitidine; (30) digestants, such as pancrelipase; (31) prokinetic agents, such as erythromycin; (32) ester local anesthetics, such as benzocaine and procaine; (33) musculoskeletal corticosteroid anti-inflammatory agents, such as beclomethasone, betamethasone, cortisone, dexamethasone, hydrocortisone, and prednisone; (34) musculoskeletal anti-inflammatory immunosuppressives, such as azathioprine, cyclophosphamide, and methotrexate; (35) musculoskeletal nonsteroidal anti-inflammatory drugs (NSAIDs), such as diclofenac, ibuprofen, ketoprofen, ketorlac, and naproxen; (36) minerals, such as iron, calcium, and magnesium; (37) vitamin B compounds, such as cyanocobalamin (vitamin B12) and niacin (vitamin B3); (38) vitamin C compounds, such as ascorbic acid; and (39) vitamin D compounds, such as calcitriol.

### Anti-Cancer BioNVs

In aspects, the present disclosure includes BioNVs. BioNVs are approximately 20-1200 nm in size which contain outwardly facing, membrane-embedded CARs capable of binding a target molecule. Without wishing to be bound by theory, the biomimetic quality owes to the nanovesicle composition which originates from the plasma membrane of allogeneic, hypoimmunogenic modified cells. In embodiments, BioNVs comprise plasma membrane-derived lipid bilayers, fully encapsulating an aqueous core which can house a variety of cell-derived molecules, including perforins, granzymes, cytokines, etc. In embodiments, the aqueous core of the NVs can further enclose exogenous biologics, fluorescent proteins, tracing dyes, radionuclides, and small molecules, among other therapeutic agents.

To ensure proper directionality of CARs and to eliminate BioNVs lacking CARs, in embodiments, HPLC-based affinity chromatography techniques can be used to select and concentrate only the BioNVs with a sufficient surface concentration of solvent-exposed CARs. HPLC-based affinity chromatography techniques can be used to reduce the concentration of contaminating cell material and NVs which harbor immunogenic cell surface markers, either by positive or negative selection.

CAR constructs may comprise a variety of structural molecules. The structure-function of a prototypical CAR includes a fusion protein comprising an extracellular (or outwardly facing) binding moiety (e.g., scFv), connected by a hinge peptide (e.g., CH2/CH3 domains from an IgG Fc region, Gly-Gly-Ser peptide linkage, CD28 peptide, CD8α peptide, etc.) to a transmembrane domain (e.g., CD28, CD3ζ, CD4, CD8α, ICOS, etc.), followed by a variety of intracellular signaling domains (e.g. 4-1BB, CD3ζ, CD28, 4-1BB, ICOS, CD27, OX40, etc.). In embodiments, BioNVs lack the intracellular machinery of whole cells and therefore the CAR design does not necessitate any intracellular signaling molecules (primary CAR construct). In embodiments, the CAR construct includes an extracellular scFV binding moiety fused with an IgG CH2/CH3 linker to a CD28 transmembrane domain and substantially lacks any intracellular domains or functionality. In embodiments, the CAR constructs have the prototypical intracellular domains swapped or otherwise fused to anchor proteins, e.g., PLA2 domain from an AAV, fusion proteins, radionuclide-binding domains, cytoskeletal elements, small molecule transporting domains, etc., which may aid in the fusion to target cells and/or packaging and release of therapeutic payloads.

In embodiments, CAR antigen-binding molecules comprise a variety of binding moieties, including antibody-based or antibody format binding domains. In embodiments, BioNVs comprise antibody or antibody format binding moieties selected from one or more of a monoclonal antibody, polyclonal antibody, antibody fragment, Fab, Fab′, Fab′-SH, F(ab′)2, Fv, single chain Fv (scFv), diabody, nanobody, linear antibody, bispecific antibody, multi-specific antibody, chimeric antibody, humanized antibody, human antibody, and fusion protein comprising the antigen-binding portion of an antibody. In embodiments, the CAR construct includes binding moieties with a Bispecific T cell Engager (BiTE), variable heavy chain IgG fragment VHH, VNAR, or through an engineered T-Cell Receptor (TCR). In embodiments, the binding moiety can be a viral epitope recognition receptor (VERR) derived from one or more oncolytic viral receptors and/or a viral ligand.

In embodiments, BioNVs are formed by disrupting the cell membranes of engineered iPSCs. In embodiments, the hypo-iPSCs are characterized by a B2M−/−, CIITA−/−, CD47+/+, PD1−/− plasma membrane profile and may be used to generate the present BioNVs. Hypo-BioNVs can be generated from the parent iPSC cell line via sonication, adaptive focused acoustics technology, French press, extrusion, serial extrusion, cell lysis by detergent, and electroporation, among other methods. In embodiments, serial extrusion is the method used to generate Hypo-BioNVs. Serial extrusion of iPSCs can produce BioNVs that are HLA1/HLA2 negative (hypoimmunogenic) with tgCD47+, exhibiting PD1 resistance elimination.

In embodiments, BioNVs can be analyzed for homogeneity of size by dynamic light scattering (DLS), flow cytometry, mass photometry, among other methods of determining particle size. In embodiments, BioNVs can be filtered for a particle size, or range of sizes, to optimize renal clearance and other clinically-relevant NV properties. In embodiments, BioNVs are about 20 nm to 1200 nm in size. In embodiments, BioNVs are about 10 nm in size, about 20 nm in size, about 30 nm in size, about 40 nm in size, about 50 nm in size, about 60 nm in size, about 70 nm in size, about 80 nm in size, about 90 nm in size, about 100 nm in size, about 120 nm in size, about 140 nm in size, about 160 nm in size, about 180 nm in size, about 200 nm in size, about 300 nm in size, about 400 nm in size, about 500 nm in size, about 600 nm in size, about 700 nm in size, about 800 nm in size, about 900 nm in size, about 1000 nm in size, about 1100 nm in size, or about 1200 nm in size. BioNVs range in size from about 10 nm to 20 nm in size, about 20 nm to 30 nm in size, about 30 nm to 40 nm in size, about 40 nm to 50 nm in size, about 50 nm to 60 nm in size, about 60 nm to 70 nm in size, about 70 nm to 80 nm in size, about 80 nm to 90 nm in size, about 90 nm to 100 nm in size, about 10 nm to 100 nm in size, about 100 nm to 200 nm in size, about 200 nm to 400 nm in size, about 400 nm to 600 nm in size, about 600 nm to 800 nm in size, about 800 nm to 1000 nm in size, or about 1000 nm to 1200 nm in size.

iPSC-derived BioNVs, in embodiments, include NVs with an outer plasma membrane leaflet only, an inner plasma membrane leaflet only, and/or both leaflets of a plasma membrane lipid bilayer intact. iPSC-derived NVs, in embodiments, include additional lipid additives (e.g., phosphatidylethanolamine, phosphatidylcholine, phosphatidylinositols, ceramides, lecithin, etc.), non-ionic surfactants (e.g., sorbitan monostearate, octadecylamine, etc.), sterols (e.g., cholesterol, bile salt derivatives, etc.), polyols (e.g., maltodextrin, sorbitol, sucrose, mannitol, etc.) and proteins (e.g., serum albumin, etc.) added for improved physicochemical properties, such as thermal stability and therapeutic payload packaging/release. The amount of cholesterol and the length and saturation of the hydrocarbon chains of the phospholipids can affect the rigidity and the stability of the bilayer, and in turn the capability of the NVs to host and release drugs, biomolecules, and other therapeutic payloads. In embodiments, BioNVs also incorporate zwitterionic lipids and methods of using zwitterionic lipids, for example, as described in US Patent Publication No. US 20130216607, the contents of which are herein incorporated by reference in its entirety. Correspondingly, functionalization of the hydrophilic heads of the lipids with polymers or biomolecules can provide additional features to the vesicle surface, thus shaping their interaction with blood components, tissues, and the immune system in vivo.

In embodiments, CAR targets include a variety of cell surface markers, including markers associated with a specific cancer, where the BioNVs are cancer-specific.

In embodiments, BioNVs are configured to encapsulate a variety of therapeutic payloads. In embodiments, primary targeted BioNVs can be used to deliver small molecule therapeutic payloads. In embodiments, second generation (or 3^{rd} or 4^{th} gen) CAR-containing BioNVs derived from activated lymphocytes can contain cytokines and other cytotoxic peptides. In embodiments, BioNVs can be formatted to encapsulate and deliver plasmid DNA, for example, to express gene editing nucleases and gRNA in target cells. Alternatively, or additionally, in embodiments, BioNVs can encapsulate the nucleases and gRNA. In embodiments, targeted second generation (or 3^{rd} or 4^{th} gen) BioNVs can be designed to encapsulate and deliver additional therapeutic proteins or peptides of interest.

In embodiments, BioNVs deliver a gene editing payload comprising a transactivating response region (TAR) loop system. In embodiments, the BioNV encapsulates a plasmid which expresses a gene editor and contains a TAR loop sequence between the 5′ end of the promoter and the gene editor/guide cassette and acts as a barrier, blocking transcription. In embodiments, transcription will only trigger in cells that are infected and contain the HIV Tat protein. In embodiments, the Tat protein binds to the TAR Loop, relaxes it, and frees the promoter for transcription, thereby expressing the editor and its guides.

In embodiments, the BioNVs are CD34+, or derived from CD34+ cells, such as human CD34+ cord blood. CD34+ cord blood-derived cell lines may serve as a base cell line for BioNV development, production, and manufacturing for the delivery of gene editing therapeutics. CD34+ cord blood-derived hypo-immunogenic cell line has been experimentally confirmed for the low expression of HLA 1/2 and overexpression of CD47 (Deuse T. et al. Hypoimmunogenic derivatives of induced pluripotent stem cells evade immune rejection in fully immunocompetent allogeneic recipients. Nat Biotechnol. 2019 March; 37 (3): 252-258).

In embodiments, the BioNVs and/or compositions comprising BioNVs are administered in combination with one or more additional compounds. In embodiments, the BioNVs are pretreated with one or more additional compounds, for example prior to administration to a subject.

In embodiments, BioNVs are modular and allogeneic (off-the-shelf) due to the lack of immunogenicity from engineered iPSCs. In embodiments, the lack of whole cell signaling components allows BioNVs to be easily tunable for target specificity and resistance to immunosuppressive signals. In embodiments, BioNVs lack the genetic elements that contribute to runaway cytokine storms, minimizing patient risk of cytokine release syndrome (CRS). In embodiments, the amounts of active cytokine, perforin, granzymes, interferon, interleukins, etc., encapsulated within the BioNV are regulated during upstream (pre-BioNV derivation) cellular processes. In embodiments, BioNVs are derived from cells capable of crossing biological barriers and/or viral receptors known for facilitation crossing.

Without wishing to be bound by theory, BioNVs generated from iPSC engineered allogeneic base cell lines represent immune invisible BioNVs which have the potential for multi-dosing. BioNV antibody-mediated neutralization is minimized, and immune cell-mediated clearance is evaded (T cell and macrophage). In embodiments, BioNVs do not contain viable genetic material from the cells they were derived to cause CRS or teratoma. In embodiments, increased expression of certain cytokines encapsulated within a BioNV can recruit natural T cells. In embodiments, BioNVs can be derived from modified cell types with or without barrier penetrating ligands to further control activity post-infusion.

### Methods of BioNV Formation

In embodiments, the modified cell is a hypoimmunogenic cell derived from iPSCs that have been engineered to have reduced or ablated expression and/or activity of immunogenic proteins and/or express or have increase expression of immunoprotective proteins. In embodiments, iPSCs are reverted from a somatic state using microRNA technology in lieu of small molecule trans-activators. The use of microRNA provides a tighter differentiation system and that results in higher quality iPSCs. Without wishing to be bound by theory, these high quality iPSCs are less prone to expression dampening (of post-engineered proteins, such as CD47) and genetic drift, and possess higher culture splitting qualities/quantities (the cultures can be divided more times than other methods before cellular integrity issues occur).

In embodiments, BioNVs derived from iPSC-derived hypoimmunogenic cells which retain the functionality from the hypoimmunogenic cell, for example and without limitation, the ability to cross the blood-brain barrier, such as is the case of macrophages/monocytes, or tissue-specific factors such as is the case in cardiomyocytes, hepatocytes, etc.

In embodiments, allogeneic iPSCs have their MHC class I and MHC class II complexes disrupted by knocking out critical proteins involved in their expression, for example, B2M which is a serum protein found in association with the MHC class I heavy chain on the surface of nearly all nucleated cells that is involved in the presentation of peptide antigens to the immune system.

In embodiments, once the B2M knock-out (KO), CIITA KO, IL-6 KO, and CD47tg knock-in (KI) is engineered into the iPSC, the TRAC and TRBC genes can be knocked out. In embodiments, only one gene for each is knocked out rather than both of on the separate alleles. In embodiments, the TRAC and TRBC genes can be knocked out as described herein. The purpose of knocking out the TRAC and TRBC genes is to eliminate the T-cell receptors. In embodiments, the modified cell is differentiated to a T-cell subset which lacks T-cell receptors to derive the BioNVs. Genetically modifying the cells to substantially lack TCRs reduces the chances for a competing ligand to the CAR construct that can target non-specifically to alternate tissues. Therefore, in embodiments, the TCR genes are knocked-out as a strategy to reduce off-target effects of the BioNVs. In embodiments, TRAC/TRBC knock-outs decrease the likelihood of CRS, as well as BioNV toxicity, generally.

In embodiments, the modified cell is expanded after engineering; any small-scale expansion or large-scale feeder system expansion methods known in the art can be used.

In embodiments, after the B2M KO, CIITA KO, IL-6 KO, CD47tg KI, and an IL-2 promoter-driven green fluorescence protein (GFP) (IL-2p GFP) reporter is constructed, the CAR constructs can be integrated/engineered into the cell. In embodiments, the CAR constructs can be knocked-in to the TRAC/TRBC genes, simultaneously knocking-out the remaining TRAC/TRBC genes, resulting in a cell that is CAR+ and TRAC/TRBC−/−. In embodiments, the CAR construct can be knocked-in to the TRAC/TRBC gene location on both loci simultaneously, resulting in a cell that is CAR+/+ and TRAC/TRBC−/−.

In embodiments, once the B2M KO, CIITA KO, IL-6 KO, CD47tg KI, IL-2p GFP KI, and CAR modified cells (e.g., iPSCs) are engineered, the Immunological Synapse (IS) quality is measured between the CAR recognition domains and the biomarker. In embodiments, the quality of the IS of BioNVs can be directly related to efficacy in whole cell therapies.

In embodiments, the BioNVs, or the hypoimmunogenic cell derived therefrom, comprises a nucleic acid encoding GFP (among other fluorescence proteins). In embodiments, once the B2M KO, CIITA KO, CD47tg KI, IL-6 KO, TRAC/TRBC single KOs are engineered into the iPSC, a GFP molecule is engineered into the modified cell line. In embodiments, this serves as the control cell line. In embodiments, the non-control cell line (the therapeutic cell line) does not have GFP. In embodiments, the nucleic acid encoding GFP is operably linked to a promoter from one or more of IL-2, perforin, granzyme, alarmin, TNF, INF, a combination thereof, and/or any other cell-specific gene or reporter gene. The IL-2 promoter is constitutively activated when lymphocytes are broadly/globally activated from various stimuli. In embodiments, a more focused activation/repression (regulation) is used. In embodiments, the IL-2p GFP reporter gene serves as an indicator for the degree of broad/global activation of the cell (as part of the BioNV derivation process). In embodiments, the GFP signal, coupled with immunoblot analysis of cytokine levels (such as perforins, granzymes, alarmins, TNFs, and INFs) allows efficient regulation of the degree of broad/global activation of a lymphocyte when exposed to activating antigens. In embodiments, GFP is used to compare the degrees of activation between manufacturing lots and ensure consistency for therapeutic development.

In embodiments, the hypoimmunogenic cells are CD34+, or derived from CD34+ cells, such as human CD34+ cord blood. In embodiments, CD34+ cord blood-derived cell lines may serve as a base cell line for BioNV development, production, and manufacturing for the delivery of gene editing therapeutics. In embodiments, a CD34+ cord blood-derived hypoimmunogenic cell line has been experimentally confirmed for the low expression of HLA 1/2 and overexpression of CD47 (Deuse et al. “Hypoimmunogenic derivatives of induced pluripotent stem cells evade immune rejection in fully immunocompetent allogeneic recipients.” Nat. Biotechnol. Vol. 37, No. 3, 2019: pp. 252-258. doi:10.1038/s41587-019-0016-3).

In embodiments, the hypoimmunogenic cell can be engineered using multiple hypoimmunogenic engineering techniques, for example, as described in Deuse et al., Han et al., Xu et al., and Harding et al., and also as described in published U.S. patent applications US20190376045, 20190376045, 20210308183, and 20210292715 to Deuse, US20210161971 to Nagy, US20180141992 to Strominger, and Published European patent application 3693384 to Poirot, each of which is incorporated by reference herein in their entirety (Han X, et al. “Generation of hypoimmunogenic human pluripotent stem cells.” PNAS. Vol. 116, No. 21 2019: pp. 10441-10446. doi: 10.1073/pnas. 1902566116.), (Xu H, et al. “Targeted Disruption of HLA Genes via CRISPR-Cas9 Generates iPSCs with Enhanced Immune Compatibility.” Cell Stem Cell. Vol. 24, No. 4, 2019: pp. 566-578. doi: 10.1016/j.stem.2019.02.005.), and (Harding et al., “Induction of long-term allogeneic cell acceptance and formation of immune privileged tissue in immunocompetent hosts.” BioRxiv 716571 [Preprint], Jul. 30, 2019. doi:10.1101/716571.).

In embodiments, BioNVs are derived from cells which have eliminated HLA genes that encode the MHC membrane glycoproteins that confer immune reactions associated with GVHD rejections. The HLA gene clusters can be divided into three categories: 1) the MHC Class I pathway, 2) the MHC Class II pathway, and 3) the MHC Class III pathway. Only the MHC Class I and II pathways express the protein complexes elicit an immune response in GVHD, whereas MHC Class III complexes are not involved in immunization activities.

The elimination of the MHC classes of protein complexes can trigger NK cells and macrophages into an active clearance mode where the cells are subsequently destroyed. To avoid this kill mechanism, in embodiments, the addition of a CD47 isoform 2 transmembrane molecular protein tag can be engineered into the cell membrane of the modified cell to avoid NK and macrophage-mediated kill responses, for example, as described in Willingham et al., Deuse et al., and Han et al. (Willingham S B, et al. “The CD47-signal regulatory protein alpha (SIRPa) interaction is a therapeutic target for human solid tumors.” PNAS. Vol. 109, No. 17, 2012: pp. 6662-7. doi: 10.1073/pnas.1121623109.). In embodiments, cells can be engineered to use additional mechanisms to prevent these responses such as those described in: 1) the CD24 transmembrane molecular protein tags (for example as performed in Zhao et al.) (Zhao W, et al. “Strategies for Genetically Engineering Hypoimmunogenic Universal Pluripotent Stem Cells.” iScience. Vol. 23, No. 6, 2020:101162. doi: 10.1016/j.isci.2020.101162.), 2) the membrane-bound surfactant protein-D (SP-D) (for example as performed in Jiaravuthisan et al.) (Jiaravuthisan P, et al. “A membrane-type surfactant protein D (SP-D) suppresses macrophage-mediated cytotoxicity in swine endothelial cells.” Transpl Immunol. Vol. 47, 2018: pp. 44-48. doi: 10.1016/j.trim.2018.02.003.), and 3) the molecular PD-L1 tag for prevention of T-cell responses. In embodiments, a BioNV derived from an ‘activated’ cell would encapsulate and/or release perforin and/or granzyme, resulting in targeted cell death. In embodiments, the activated cell would generate perforin and/or granzyme to be packaged into the BioNV. In embodiments, hypoimmunogenic cells that are to be activated would not express PD-L1 to avoid the resultant BioNV from being targeted to PD-1 on T-cells. In embodiments, this reduces the likelihood of releasing perforin and/or granzyme, resulting in unwanted T-cell death. In embodiments, PD-L1 is overexpressed in BioNVs derived from a cell that has not been activated and is not loaded with apoptotic cytokines. In embodiments, hypoimmunogenic cells that are to be activated have PD-L1 downregulated, knocked-out, or otherwise silenced. In embodiments hypoimmunogenic cells that are not to be activated have PD-L1 upregulated, i.e. for BioNVs used for gene editor delivery. In embodiments, CD47 can be utilized in genetically engineered iPSCs for immune tolerance to innate immune cells, for example, such as in Chhabra et al., Han et al., and Jaiswal et al. (Chhabra A, et al. “Hematopoietic stem cell transplantation in immunocompetent hosts without radiation or chemotherapy.” Sci Transl Med. Vol. 8, No. 351, 2016: 351ra105. doi: 10.1126/scitranslmed.aae0501.) and (Jaiswal S, et al. “CD47 is upregulated on circulating hematopoietic stem cells and leukemia cells to avoid phagocytosis.” Cell. Vol. 138, No. 2, 2009: pp. 271-85. doi: 10.1016/j.cell.2009.05.046.). In embodiments, cells can be modified as described in U.S. Pat. No. 8,562,997 to Jaiswal, et al., which is incorporated by reference herein in its entirety.

In embodiments, some approaches can be used which do not entirely knock-out all HLA genes, for example, as performed in Xu et al. and Han et al., which only knock-out the HLA genes that are highly associated with an immune response, leaving intact the HLA genes that dampen a macrophage or NK response (e.g., HLA-E, HLA-F, and HLA-G). In embodiments, this approach does not require the addition of a CD47 tag; the modified cell can be engineered to generate BioNVs with or without CD47.

In embodiments, methods improve upon the approaches of hypoimmunogenicity of Table 3.

TABLE 3
Three methods of modification of cells using the HLA knockout combined with a CD47 isoform
2 tag and a PD-L1 transmembrane tag (Zhao, et al. 2020) (Gornalusse G G, et al. “HLA-
E-expressing pluripotent stem cells escape allogeneic responses and lysis by NK cells.”
Nat Biotechnol. Vol. 35, No. 8, 2017: pp. 765-772. doi: 10.1038/nbt.3860.).

| Source | HLA-A | HLA-B | HLA-C | HLA-E | HLA-F | | HLA-G | HLA-II | CD47 | PD-L1 |
|---|---|---|---|---|---|---|---|---|---|---|
| Gornalusse, | Knockout | | | + | | Knockout | | | | |
| 2017 | | | | | | | | | | |
| Deuse, | | B2M Knockout | | | | | | CIITA KO | + | |
| 2019 | | | | | | | | | | |
| Xu, | Knockout | | | | | | | CIITA KO | | |
| 2019 | | | | | | | | | | |
| Han, | Knockout | | | | | | | CIITA KO | + | + |
| 2019 | | | | | | | | | | |

In embodiments, developing the allogeneic modified cell involves the removal of MHC Class I and MHC class II protein complexes through the disruption of certain HLA genes, or a B2M knockout, followed by knocking out the CIITA gene. In embodiments, the knockouts can be performed using CRISPR gene editing approaches, due to their rapid mechanism of action. In embodiments, the knockouts are performed using Zinc Finger Nucleases (ZFNs) and/or TALENS. In embodiments, Cre/Lox recombinase systems are used to generate the modified cell. In embodiments, RNA silencing (RNAi, shRNA, microRNA, CRISPR Cas13a-d, etc.) is used to generate the modified cell.

In embodiments, the methods of developing the allogeneic, hypoimmunogenic modified cell is distinct from the methods of creating allogenicity of Harding et al. In lieu of deleting the MHC class I/II genes and running the risk of preventing long-term acceptance by the recipient, the Harding et al. method includes an alternate approach based on immune escape mechanisms that occurs in nature. The method relies on the Harding et al. biomimicry based on the horizontally transmitted cancer, DFTD type 2, that is predominant in Tasmanian devils. In embodiments, developing the allogeneic modified cell can include expression or increased expression of the immunomodulatory proteins CCL21, PD-L1, FasL, SerpinB9, H2-M3, CD47, CD200, and/or MFG-E8 to protect cell derivatives from long-term immune rejection in mice (and humans), without the deletion of MHC class I/II proteins. In embodiments, the modified cell expresses one or more of the proteins shown in Table 4, including any splice variant and/or isoform of any of the indicated proteins (e.g., CD200 splice variants). In embodiments, this system can be used to interfere with the activity of APCs, macrophages, NK cells, and T-lymphocytes. In embodiments, the modified cell lines can also contain the safe-cell system developed by Liang et al., where cell division genes are linked to a suicide gene to prevent runaway teratomas leading to cancers (Liang Q, et al. “Linking a cell-division gene and a suicide gene to define and improve cell therapy safety.” Nature. Vol. 563, No. 7733, 2018: pp. 701-704. doi: 10.1038/s41586-018-0733-7.).

In embodiments, methods improve upon the approaches of hypoimmunogenicity of Table 4.

TABLE 4
Expression or increased expression of illustrative
proteins for creating allogeneic modified cells.

| | | Illustrative |
|---|---|---|
| | | Immunomodulatory |
| Gene | Type of protein | Function |
| Ccl2 | Chemokine | Recruitment of |
| | | activated DCs |
| PD-L1 | Check Point Inhibitor | Shuts Down Effector T-cell |
| (non-activated | | Responses |
| cells and/or | | |
| BioNVs) | | |
| FASL | Transmembrane Signal | Induces T-cell Apoptosis |
| H2-M3 | Antigen Presentation - | Inhibits NK cells |
| | MHC-like | |
| SERPINB9 | Inhibitory Chemokine | Blocks granzyme B |
| | (Serine Protease Inhibitor) | function |
| Cd47 | Transmembrane SIRP-αR | Blocks phagocytosis |
| | ligand | |
| Cd200 | Type 1 Membrane | Prevents macrophage and |
| | Glycoprotein | granulocyte activation |
| Mfg-e8 | Chemokine (attractant) | Macrophage recruitment to |
| | | anti-inflammatory |
| | | profile site |

In embodiments, hypoimmunogenic cells from which BioNVs are derived are engineered to have knock-outs of one or more of HLA-A, HLA-B, HLA-C, HLA-E, HLA-G, HLA-F, CIITA, IL-6, IL-4, IL-10, IL-16, TRAC, TRBC, SerpinB9, and/or any combination thereof; and knock-ins of one or more of CCL2, PD-L1 (in BioNVs derived from non-activated cell sources), CTLA-4, H2-M3, CD24, CD47 (minus the 3′ UTR region or an alternate 3′ UTR region that does not contain binding sites for the inhibitory microRNAs), MFG-E8, CD200, and/or any combination thereof.

In embodiments, BioNVs are generated from a hypoimmunogenic cell with one or more of the modifications of Table 5.

TABLE 5
Illustrative engineered cell expression profile for BioNV formation
for human use (Fife B T and Bluestone J A. “Control of peripheral
T-cell tolerance and autoimmunity via the CTLA-4 and PD-1
pathways.” Immunol Rev. Vol. 224, 2008: pp. 166-82. doi:
10.1111/j.1600-065X.2008.00662.x.) and (Rong Z, et al. “An
effective approach to prevent immune rejection of human ESC-
derived allografts.” Cell Stem Cell. Vol. 14, No. 1 2014:
pp. 121-30. doi: 10.1016/j.stem.2013.11.014.).

| | Knock-ins (inducible or constitutive |
|---|---|
| Knockouts | over-expression) |
| HLA-A | CCL2 |
| HLA-B | PD-L1 (non-activated cells and/or |
| | BioNVs) and/or CTLA-4 (Fife et al. |
| | and Rong et al.) |
| HLA-C | H2-M3 |
| HLA-E or HLA-G (but not both) | CD24 or CD47 or CD47 tethered to |
| | CD24 |
| HLA-F | MFG-E8 |
| CIITA | CD200 for non-immunomodulatory |
| IL-6 | functions only (such as liver |
| | cell regeneration). These cells |
| | will not contain a CD47 tag. |
| IL-4, IL-10, or IL-16 (optional) | SerpinB9 |
| | (where granzyme is not used) |
| TRAC | |
| TRBC | |
| SerpinB9 | |
| (where granzyme is used) | |

In embodiments, inactivation/activation of genes is controlled by inducible promoters throughout the differentiation, activation, and manufacturing process for BioNVs. In embodiments, disruption of MHC, TCR, and CRS genes produce allogeneic iPSCs which are −/− CRS and −/− TCR, leading them to have plasma membranes which exhibit hypoimmunogenic properties upon infusion into a subject. CRS genes implicated in the pathogenesis of CRS include IL-6, IL-10, IFN-γ, monocyte chemoattractant protein 1 (MCP-1), granulocyte-macrophage colony-stimulating factor (GM-CSF), among other cytokines, including tumor necrosis factor (TNF), IL-1, IL-2, IL-2-receptor-α, and IL-8. In embodiments, one or more of these genes is inactivated, e.g., in a cell from which the BioNVs are derived.

In embodiments, BioNVs are formed by disrupting the cell membranes of engineered iPSCs. In embodiments, the hypo-iPSCs are characterized by a B2M−/−, CIITA−/−, CD47+/+, PD1−/− plasma membrane profile and may be used to generate BioNVs. Hypoimmunogenic BioNVs can be generated from the parent iPSC cell line via sonication, adaptive focused acoustics technology, French press, extrusion, serial extrusion, cell lysis by detergent, and electroporation, among other methods. In embodiments, serial extrusion is the method used to generate hypoimmunogenic BioNVs. In embodiments, serial extrusion of iPSCs can produce BioNVs that are HLA1/HLA2 negative (hypoimmunogenic) with tgCD47+, exhibiting PD1 resistance elimination.

In embodiments, genetic engineering of iPSCs includes gene-editing techniques such as CRISPR-based gene editing systems, zinc finger nucleases (ZFNs), transcription activator-like effector nuclease (TALEN), meganucleases, among other gene editing methods, for the purpose of generating allogeneic/hypoimmunogenic iPSCs and/or for CAR cassette integration. In embodiments, the genetic engineering of iPSCs can refer to the decrease or ablation of transcription of any genetic element; likewise, genetic engineering of iPSCs can refer to the increase in the expression of or the knock-in of any genetic element, including both endogenous and exogenous genetic elements.

For example, in embodiments, stable cell integration (safe harbor genetic location) in iPSCs can be controlled by implementing a Tet-regulated CRISPRa+targeted 3× transcription factor targeted gRNA system. The CRISPR activation system for three upstream transcription factors can trigger a signal cascade event that enhances the production of CARs that have replaced endogenous antibody ORFs at designated locus (loci). This system can be ‘tunable’ by including a Tet-regulated promoter, allowing for the ability to vary the concentrations of CARs on the surface of the cell. Next, stable cell replacement of CDRs and heavy and light antibody regions with CAR cassettes can be achieved via Cpf-1 directed homology directed repair (HDR). Finally, the stably integrated CAR cassette can contain flanking gRNA binding sites which allow the scFV (among other antibody formats) or VERR/viral ligand to be repeatedly swapped or altered for rapid and consistent insertion of a desired sequence.

In embodiments, the allogeneic and hypoimmunogenic properties of the iPSCs can be further improved by inducing overexpression of immunoprotective molecules. For example, and without limitation, overexpression of CD47—among other cell surface integrins—can decrease the kinetics of macrophage depletion of BioNV products from the blood. In embodiments, additionally, allogeneic and hypoimmunogenic properties of iPSCs can be improved by expression of α- and β-phagocytic integrins. In embodiments, overexpression of similar immunogenically protective cell surface markers which signal to leukocytes can be performed as a strategy to increase the half-life of BioNVs post-infusion.

In embodiments, iPSCs are genetically engineered for CAR cassette integration. CAR cassette integration can include both integrative and non-integrative transgene insertion. Non-limiting examples of non-integrative transgene insertion include mRNA, non-integrative lentivirus, and endonuclease-targeted methods. Integrative CAR cassette insertion methods include stable retroviral vector insertion and transposase-based integration systems. Stable CAR cassette transduction can be achieved, for example, using retroviral vectors which can enable iPSCs to maintain the genetic element encoding the CAR throughout differentiation, expansion, and activation. In embodiments, clinical-grade, stable transduction of CAR cassettes into T cells has been achieved for brexucabtagene autoleucel (Tecartus®, Kite Pharma Inc.) and axicabtagene ciloleucel (Yescarta®, Kite Pharma Inc.) using GRV vectors, while tisagenlecleucel (Kymriah®, Novartis International AG) is transduced using a lentiviral vector (Labbé, R. P., et al. “Lentiviral Vectors for T Cell Engineering: Clinical Applications, Bioprocessing and Future Perspectives.” Viruses 13 (2021); 1528. doi: 10.3390/v13081528).

In embodiments, the concentration of the CAR on the surface of the iPSC base cell line, or any downstream differentiated cell (and the resulting BioNVs), can be regulated using a variety of transcription control elements, such as a tetracycline on/off promoter (or similar drug-regulated promoters) to drive the expression of a CRISPR activation/gRNA (CRISPRa) system. The CRISPRa system can then activate the antibody-regulating transcription factors, for example, Drm2, Fr5, and Bxp2, which regulate the expression of an engineered CAR cassette that has been integrated at the site of an antibody locus (where the antibody genes have been replaced). Additionally, a similar transcription control element can be provided to control overexpression of genes (e.g., CD47), drive genes controlling differentiation, etc., at defined manufacturing stages.

Subjects and/or Animals

In embodiments, the subject and/or animal is a mammal, e.g., a human, mouse, rat, guinea pig, dog, cat, horse, cow, pig, rabbit, sheep, or non-human primate. In embodiments, the subject and/or animal is a non-mammal, for example, a zebrafish. In embodiments, the subject and/or animal is a transgenic animal comprising a fluorescent cell, such as, for example, an RPE cell and/or an immune cell with GFP. In embodiments, the subject and/or animal is a human. In embodiments, the BioNVs originate from fluorescently-tagged cells and/or are packaged with fluorescently-tagged proteins or tags (with e.g., GFP). In embodiments, the human is a pediatric human, human adult, geriatric human, an infant or child. In other embodiments, the human is referred to as a patient.

In embodiments, the method of treatment includes administering to a human who has an age in a range of from about 0 months to about 6 months old, from about 6 months to about 12 months old, from about 12 months to about 18 months old, from about 18 months to about 36 months old, from about 1 year to about 5 years old, from about 5 years to about 10 years old, from about 10 years to about 15 years old, from about 15 years to about 20 years old, from about 20 years to about 25 years old, from about 25 years to about 30 years old, from about 30 years to about 35 years old, from about 35 years to about 40 years old, from about 40 years to about 45 years old, from about 45 years to about 50 years old, from about 50 years to about 55 years old, from about 55 years to about 60 years old, from about 60 years to about 65 years old, from about 65 years to about 70 years old, from about 70 years to about 75 years old, from about 75 years to about 80 years old, from about 80 years to about 85 years old, from about 85 years to about 90 years old, from about 90 years to about 95 years old or from about 95 years to about 100 years old.

In embodiments, the subject is a non-human animal, and therefore the disclosure pertains to veterinary use. In embodiments, the non-human animal is a household pet. In embodiments, the non-human animal is a livestock animal.

In embodiments, sera and/or immune cells and/or tumor cells are evaluated and/or effected. In embodiments, immune cells include cells of a subject's and/or animal's innate immune system. In embodiments, such cells include, but are not limited to NK cell, monocyte, DC, B cell, macrophage, CD4+ T cell, and CD8+ T cell. In various embodiments, the disclosure provides for detecting a presence, detecting an absence, or measuring an amount of tumor volume, tumor cells, metastasis, cDNA, or RNA in a sample originating from a subject.

### Kits

The disclosure, in embodiments, provides kits that can simplify the administration of any agent described herein. An exemplary kit of the disclosure comprises any agent described herein in unit dosage form. In embodiments, the unit dosage form is a container, such as a pre-filled syringe, which can be sterile, containing any agent described herein and a pharmaceutically acceptable carrier, diluent, excipient, or vehicle. In embodiments, the kit further comprises a label or printed instructions instructing the use of any agent described herein. In embodiments, the kit also includes a lid speculum, topical anesthetic, and a cleaning agent for the injection surface. In embodiments, the kit further comprises one or more additional agents described herein.

In aspects, the present disclosure includes a syringe comprising one or more compositions of the present disclosure. In embodiments, the syringe is prefilled with a volume of the composition. In embodiments, the syringe is prefilled in a volume of about 1 mL to about 10 mL. In embodiments, the syringe is prefilled in a volume of about 10 mL, about 9 mL, about 8 mL, about 7 mL, about 6 mL, about 5 mL, about 4 mL, about 3 mL, about 2 mL, about 1.9 mL, about 1.8 mL, about 1.7 mL, about 1.6 mL, about 1.5 mL, about 1.4 mL, about 1.3 mL, about 1.2 mL, about 1.1 mL, or about 1.0 mL or less of the composition.

In embodiments, the syringe comprises a composition having a shelf stability ranging from about 1 hour to about 1 week. In embodiments, the syringe comprises a composition having a shelf stability of at least about 12 hours, about 24 hours, about 36 hours, about 48 hours, or about 72 hours when stored at a temperature ranging from about −85° C. to about 25° C. In embodiments, the syringe comprises a composition having a shelf stability of at least about 12 hours, about 24 hours, about 36 hours, about 48 hours, or about 72 hours when stored at a temperature ranging from about 15° C. to about 25° C.

In embodiments, the storage temperature is about −80° C. In embodiments, the storage temperature is about −20° C. In embodiments, the storage temperature is about 4° C. In embodiments, the storage temperature is about 21° C. In embodiments, the kit includes lyophilized BioNVs.

In one embodiment, the kit comprises a container containing a composition comprising BioNVs of the present disclosure, a therapeutically effective amount of an additional therapeutic agent, such those described herein, and instructions for use.

### EXAMPLES

### Example 1: Generating BioNVs Via Serial Extrusion

Biomimetic Nanovesicles (BioNVs) can be produced from hypoimmunogenic cell lines as illustrated in the scheme depicted in FIG. 17. The following protocols outline experiments for mini-CAR BioNVs. The mini-CAR BioNVs can be derived from any cell type. The following outlines BioNVs derived from either activated or non-activated Natural Killer lymphocytes (NK cells). The BioNVs can be designed to target any antigen receptor, as described herein.

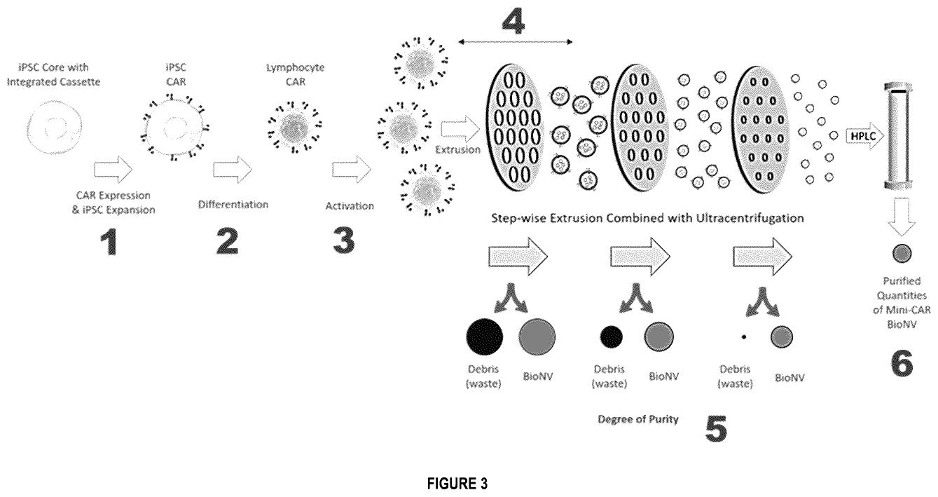

The level of CAR expression can be measured in the hypoimmunogenic cell line using a combination of flow cytometry and iodixanol density gradient (e.g., STEP 1 of FIG. 3).

The differentiation of the iPSC-expressing surface CAR into lymphocytes can be analyzed by lymphocyte marker identification including, for example CD4/CD8 (T-cells) or CD56/CD16 (Natural Killers cells), among other cell surface markers (e.g., STEP 2 of FIG. 3). The expression profile can be determined via flow cytometry, RT-PCR, and/or CRISPR-based analytics.

Next, the activation of the lymphocytes can be achieved using biomarker antigen-coated beads in low, pre-determined concentrations over the course of two weeks in two stages (e.g., STEP 3 of FIG. 3). This process can also analyze the quality of the Immunological Synapse (IS) between the CAR and the antigen-coated beads, using well-established protocols to measure i) the quantification of F-Actin accumulation at the site of synapse formation, ii) the distribution of pZeta at synapse, iii) the clustering of an antigen through the IS location, and/or iv) the polarization of lytic granules (LGs) that contain perforin and granzymes.

After the activation of the lymphocytes, the cells are expanded using established protocols (e.g., STEP 4 of FIG. 3). After expansion, the levels of perforin and granzyme (or other lumen payloads if applicable) are analyzed per cell population to ensure consistent concentration levels on a per-batch basis. This is accomplished using a series of qPCR, immunoblotting, flow cytometry, and/or mass spectrometry. The expansion step may not be necessary if a large enough cell population from Step 3 can be achieved.

Once the cells are activated to produce the desired therapeutic protein(s), they are expanded, harvested, washed several times, and then placed into a buffered extrusion medium. The cells are then wholly processed via serial extrusion through each step of the polycarbonate filter system that consists of diminishing pore size (e.g., STEP 5 of FIG. 3). In the initial extrusion step of the serial extrusion process, the nucleus (along with nuclear components including nuclear pores, genomic material, and transcription factors) and mitochondria are eliminated. The sample is then treated with endonuclease, e.g., BENZONASE. BENZONASE is a non-specific, recombinant endonuclease that cleaves all types of DNA and RNA variants into non-functional fragments<8 soluble base pairs. This leads to the highest reduction of nucleic acid load on a per sample and scalable basis and does not interfere with BioNV membrane chemistry. The cleavage process also eliminates nucleic viscosity, allowing for subsequent loading and passage of materials through the next set of extrusion filters.

The serial extrusion process will avoid the elimination of other organelles such as the Golgi Apparatus or the ER. The membrane system of these organelles is highly evolved to traffic vesicles (release and uptake) between folded membranes. For example, the cis and trans face of the Golgi Apparatus contain unique lipid compositions that facilitate low energy barrier absorption and release in the trafficking of vesicles. These components are relatively low in the cytoplasmic membrane. Therefore, isolating the cytoplasmic membrane for BioNV derivation is not as favorable. As the BioNVs are passed through the polycarbonate filters in the serial extrusion process, they undergo destruction and spontaneous formation based on the pore size. This process results in BioNVs containing membranes with a homogenous mixture of cytoplasm, Golgi, and ER lipid content and protein components that can considerably increase their affinity for cellular and tissue delivery uptake in comparison to BioNVs processed to eliminate these organelles. These features could translate to better and more consistent uptake of BioNVs into targeted cells at much lower doses than systems that do not incorporate these properties.

After the extrusion step, the BioNVs are passed through an α-CD3 HPLC (FPLC in scale-up) column to remove the low percent (approximately 0.05%) of inverted BioNVs that spontaneously form during the serial extrusion process (e.g., STEP 6 of FIG. 3). This is done to ensure the resultant BioNVs have homogenous directionality with respect to the membranes. Low loss of yield occurs during this step, as it is a flow-through process to capture impurities. Once the BioNVs have been collected after the HPLC/FPLC step, they are tested through a standardization process.

The standardization process includes one or more the following assays:

BioNV homogeneity: the use of Nanoparticle Flow Cytometry (NanoFCM) can confirm BioNV concentration, homogeneity of size, the density of the BioNVs, and/or the homogeneity of the BioNV lumen constituents.

Concentration of lumen payload: NanoFM technology can be used to determine the type and concentration of the nucleic acids/proteins that are packaged into the lumen of the BioNVs. These data can be confirmed in parallel with one or more methods including immunoblot, mass spectrometry, and BCA analyses to determine the nucleic acid and protein content of BioNVs.

BioNV Stability: a combination of nanoparticle tracking analysis (NTA), dynamic light scattering (DLS), and electron microscopy (EM) can be used in combination with immunoblot and mass spectrometry analyses to determine the physical and biochemical features of the BioNVs over 8 to 10 months. Data from these assays can include the protein expression profile, the degree of intact BioNV membranes/packaging, and/or the degree of aggregation.

Membrane Integrity: the integrity of the BioNV membranes is evaluated using calcein release assays combined with NanoFCM to assess membrane permeability. The results can provide insight into the leakage properties of the BioNVs against standardized BioNV panels.

Quality of lumen payload: the quality of the lumen-packaged payloads can be determined using multiple analytic assays, depending on the nature of the payload. In instances where the deliverable is a nucleic acid, qPCR and/or sequencing over 8 to 10 months can be used to check the integrity and quantity of the nucleic acid payloads. For proteins, an analysis of the BioNV constituents using one or more of NanoFCM, mass spectrometry, and immunoblot analyses can be used to analyze the protein payload.

CAR Quality and Surface Density: CAR surface density can be determined using NanoFCM, mass spectrometry, and/or immunoblot analyses. CAR surface density is expected to be at least about 5-fold to at least about 10-fold higher in BioNVs compared to whole cell surface densities. This could considerably enhance targeting to the antigen in comparison to a whole cell. CAR quality can be determined at the cellular stage, as described above (e.g., as in STEP 3). A mathematical model can be used to extrapolate cellular quality data and apply it to the BioNVs in relation to efficacy study data outcomes.

BioNV Functionality: BioNVs can be tested for basic functionality, including multiple and defined standardization assays, such as in vitro cellular uptake into targeted cells with and without expressed antigen, as well their ability to cross dense tissues such as those in human retinal models. Following these basic functionality assays, which can be performed immediately after the serial extrusion process, pre-clinical studies will address the remainder of the quality and functionality properties of the BioNVs.

Following the extrusion process, the mini-CAR BioNVs (as well as VERR and/or viral ligand BioNVs) will be segmented based on activation immunological synapse (IS) binding into the following groups:
1) Low quality IS pool of whole cells (activated), where “low quality” refers to degree of IS binding;
2) Moderate quality IS pool of whole cells (activated), where “moderate quality” refers to degree of IS binding relative to “low” and “high” qualities;
3) High quality IS pool of whole cells (activated), where “high quality” refers to degree of IS binding; and
4) High quality IS pool of whole cells (non-activated).

BioNVs can be cryopreserved at this stage.

Statistical analysis will be used to evaluate and address batch-to-batch consistency. Upper and lower control limits will be based upon data using controls (e.g., means, 95% confidence intervals, range, and/or percentile distribution).

### Example 2: In Vitro Evaluation of BioNVs

Experiments will be conducted in triplicate with each of the poor, moderate and high quality activated mini-CAR NK BioNVs and high quality mini-CAR NK non-activated BioNVs. Experiments will be conducted with combination with whole cell CAR NKs.

Cell Cytotoxicity: mini-CAR NK BioNVs (and whole cell mini-CAR NK cells) will be evaluated for cell cytotoxicity using a Chromium-51 (⁵¹Cr) release assay using multiple types of cell lines derived from various cancers alongside control cell lines. Target cells will be cultured with ⁵¹Cr which will be integrated within the cell. After treatment of cancer cell lines with BioNVs (and/or whole cells) the ⁵¹Cr is then released from the target cells by during lysis. Supernatants can be collected, centrifuged to clarify the supernatant, and the amounts of ⁵¹Cr can be measured as a proxy for cell lysis. ⁵¹Cr can be measured directly using a gamma counter, or mixed with a scintillation cocktail to be read in a plate reader (e.g., using a LUMAPLATE, Perkin Elmer).

Nanoparticle flowcytometry (NanoFCM), ELISA, and/or immunoblot analysis will be used to monitor the concentrations and presence of the lumen-loaded therapeutic payloads, such as perforins, granzymes, p53, alarmins, TNFs, and/or INFs. The concentrations and/or presence of these proteins can also be assayed during cell killing assays. PCR will be used to monitor the activation state of apoptotic-associated genes, such as BAD, BAX, and Caspase-3 during the course of treatment. The uptake of each subset of mini-CAR NK BioNV into the cells will be measured by rhodamine staining. A control treatment with “high quality” non-activated mini-CAR NK BioNVs will be used for comparison.

Evaluation will be performed in one or more of the following cell lines.

### Lung Cancer Cell Lines:

A549: widely used non-small cell lung cancer (NSCLC) cell line derived from a lung carcinoma.

H1975: NSCLC cell line derived from a patient with an acquired resistance to the epidermal growth factor receptor (EGFR) inhibitor, gefitinib.

H1650: NSCLC cell line that carries an EGFR exon 19 deletion mutation, which is associated with sensitivity to EGFR tyrosine kinase inhibitors (TKIs).

HCC827: NSCLC cell line that harbors an EGFR exon 19 deletion mutation and is highly sensitive to EGFR TKIs.

H1299: A p53-null NSCLC cell line frequently used in lung cancer research.

Calu-3: A lung adenocarcinoma cell line derived from a metastatic site.

PC-9: NSCLC cell line derived from a lung adenocarcinoma patient with an EGFR exon 19 deletion. PC-9 cells are used to investigate EGFR-targeted therapies.

### Hepatocellular Carcinoma Cell Lines:

HepG2: liver cancer cell line.

Huh7: hepatocellular carcinoma cell line.

PLC/PRF/5: liver cancer cell line for investigating viral hepatitis-related HCC.

Hep3B: hepatocellular carcinoma cell line that lacks functional p53.

SK-Hep-1: hepatocellular carcinoma cell line derived from a metastatic site in the lymph node of a patient with liver cancer.

### Breast Cancer Cell Lines:

MCF-7: hormone receptor-positive (estrogen receptor-positive and progesterone receptor-positive).

T47D: hormone receptor-positive breast cancer cell line derived from a metastatic site of a patient with infiltrating ductal carcinoma.

MDA-MB-231: triple-negative breast cancer cell line, lacking expression of estrogen receptor, progesterone receptor, and human epidermal growth factor receptor 2 (HER2).

BT-474: breast cancer cell line that overexpresses the HER2/neu receptor.

SK-BR-3: HER2-positive breast cancer cell line derived from the pleural effusion of a patient with metastatic breast adenocarcinoma.

ZR-75-1: estrogen receptor-positive breast cancer cell line derived from a metastatic site of a patient with infiltrating ductal carcinoma.

### Brain Cancer Cell Lines:

U87MG: glioblastoma cell line.

U251: glioma cell line derived from a human glioblastoma multiforme tumor.

LN-18: glioma cell line derived from a human glioblastoma used for studying glioma invasion.

T98G, SF268, D54, A172: glioblastoma cell lines derived from a human glioblastoma multiforme tumor.

### Renal Cell Carcinoma Cell Lines:

786-O: renal cell carcinoma cell line derived from a primary renal tumor of a patient with clear cell carcinoma.

ACHN: renal cell carcinoma cell line derived from a human renal adenocarcinoma.

Caki-1, 769-P: renal cell carcinoma cell lines derived from a human clear cell carcinoma of the kidney.

A498: renal cell carcinoma cell line derived from a human renal cell carcinoma with a clear cell histology.

RCC-MF: (Renal Cell Carcinoma Metastasis to Fibula) renal cell carcinoma cell line derived from a metastatic lesion in the fibula of a patient with renal cell carcinoma.

### Lymphoma Cell Lines:

Raji, Daudi: Burkitt lymphoma cell lines that serve as a model for aggressive B-cell lymphomas.

Jurkat: T-cell lymphoma cell line derived from a patient with acute T-cell leukemia.

SU-DHL-4: diffuse large B-cell lymphoma (DLBCL) cell line.

HUT78: cutaneous T-cell lymphoma (CTCL) cell line derived from a patient with Sezary syndrome.

OCI-LY10: DLBCL cell line derived from a patient with DLBCL.

Toledo: mantle cell lymphoma cell line derived from a patient with mantle cell lymphoma.

### Leukemia Cell Lines:

K-562: chronic myelogenous leukemia (CML) cell line derived from a patient with CML in blast crisis.

HL-60: promyelocytic leukemia cell line derived from a patient with acute promyelocytic leukemia.

THP-1: monocytic leukemia cell line derived from a patient with acute monocytic leukemia.

Jurkat: T-cell leukemia cell line derived from a patient with acute T-cell leukemia.

MV4-11: myelomonocytic leukemia cell line derived from a patient with AML.

NALM-6: B-cell precursor leukemia cell line derived from a patient with B-cell acute lymphoblastic leukemia (ALL).

REH: B-cell precursor leukemia cell line derived from a patient with B-cell ALL.

### Melanoma Cell Lines:

A375, SK-MEL-28, SK-MEL-2, WM-266-4, UACC-62: melanoma cell lines derived from a metastatic site of a patient with malignant melanoma.

M14, MeWo: melanoma cell lines derived from a primary tumor of a patient with malignant melanoma.

### Example 3: In Vivo Evaluation of Mini-CAR NK Cell and BioNV Efficacy

Experiments will be conducted with mini-CAR NK whole cells and BioNVs that are anti-GPC3. GPC3 CAR NK cells will be used to establish an immunological synapse (IS) quality standard curve.

Mouse Models: one of more of the following mouse models will be used to test BioNVs and/or whole cells: hGPC3TG-NSG: a hepatocellular carcinoma (HCC) expressing human GP3 protein in immunodeficient mice lacking mature T cells, B cells, and NK cells; HCC NSG: hepatocellular carcinoma (HCC) immunodeficient mice lacking mature T cells, B cells, and NK cells; HCC PDX: hepatocellular carcinoma (HCC) patient-derived xenograft mice with tumor implantation; and Severe combined immunodeficiency (SCID) mice. Mice can have C57BL/6J backgrounds.

Tumor Implantation & Visualization: tumor cells will be fluorescently-labeled prior to administration in establishing in vivo tumors to enable visualization and measurements using an in vivo animal imaging device, such as a dual positron emission tomography (PET) scan and a computed tomography (CT) scan (PET/CT), e.g., IVIS know dose, high resolution microCT (Perkin Elmer). Tumor cells will be transduced under puromycin selection with a retrovirus encoding one or more luciferase gene, such as the RLuc luciferase gene (sea pansy, Renilla reniformis), FLuc, and teLuc. To assess the efficacy and persistence of GPC3 CAR NKs in vivo, several subsets will be administered, including the “low,” “moderate,” and “high” IS quality GPC3 CAR NKs (e.g., as described above), where the cells will be transduced with plasmid eGFP (enhanced green fluorescent protein). Mice at 8-10 weeks of age (e.g., hGPC3TG-NSG, HCC NSG, and/or HCC PDX mice) will be intraperitoneally (IP) injected with 5×10⁶ fluorescently labeled tumor cells suspended in a cancer cell suspension matrix, e.g., MATRIGEL (Becton Dickinson). At approximately 5-10 days later, when palpable tumor is detectable (approximately 50 mm²), 5×10⁶ GPC3 CAR NKs and control GCP3-CAR-NK cells will be administered via IP injected. For in vivo imaging of GPC3 CAR NKs expressing eGFP and tumor cells expressing the luciferase gene (mice will be IP injected with D-luciferin (at about 150 mg/kg, a substrate for eGFP-FLuc) and coelenterazine (at about 1 mg/kg, a substrate for RLuc), respectively. Because eGFP and RLuc utilize different substrates, they can be combined as dual reporters to monitor both tumor and GPC3 CAR NKs.

Tumors in the hGPC3TG-NSG, HCC NSG, and/or HCC PDX mouse models will be visualized and measured using PET/CT, e.g., IVIS know dose, high resolution microCT (Perkin Elmer). Alternatively, tumor size can be measured using a caliper at the greatest longitudinal diameter (length) and the greatest transverse diameter (width).

Tumor penetration: tumor penetrating properties of each IS quality of mini-CAR NK GPC3 BioNV will be evaluated at various stages of tumor growth and compared to whole cell infiltration of tumors. BioNV tumor penetration can also be compared to other systems, like oncolytic viruses, AAVs, and/or exosomes). Mini-CAR NK BioNVs (and cells) derived from the three sets described and from activated and non-activated whole cells will be tested by tumor cross-sectioning and/or immunostaining over various stages of tumor growth and time course.

Anti-tumor activity in vivo: homing, persistence, and anti-tumor activity of each quality of mini-CAR NK BioNV (separately and in combination with the whole cell CAR NK), will be evaluated in chip-based tumor models and animal models. Severe combined immunodeficiency (SCID) and patient-derived xenograft (PDX) mouse models will be used with an in vivo imaging system (e.g., PET/CT and/or IVIS, Perkin Elmer). Efficacy will be measured using one or more of tumor volumes, tumor weights, and tumor inhibition in a dose-dependent response manner over a time course. Each tumor will be monitored for the release of the mini-CAR therapeutic cargos, such as perforin, granzymes, and p53. PCR, ELISA, and/or immunoblotting will be used to evaluate apoptotic gene activation, such as BAD, BAX, and Caspase-3 during the course of treatment.

### Example 4: In Vitro and In Vivo Evaluation of CRS-Related Toxicity

Cytokine activation: the degree of in vitro cytokine activation in one or more of the cell lines outlined herein will be evaluated with each quality mini-CAR NK GCP3 BioNV and CAR NK GCP3, with one or more control CAR cell lines (e.g., CAR-T-T1E, CAR-T cells with a pan-ErbB targeted CAR). The cancer cell lines will be co-cultured with CD8 T-cells. The mini-CAR NK GCP3 BioNVs and CAR NK GCP3 cells will be added in increasing concentrations and the degree of cytokine activation will assayed over a time course. The levels of molecular IL-6, IL-2, and/or INF-γ will be measured via PCR, immunoblot, ELISA, and/or flow cytometry. In addition, cell cytotoxicity will be evaluated by ⁵¹Cr release assay, as described above.

CRS Reduction: in vivo models will be used to measure the degree of cytokine release syndrome (CRS) caused by each quality of the mini-CAR NK GCP3 BioNVs, the CAR NK GCP3 cells, and combination therapy thereof in comparison to a standardized CRS-causing, CAR-T-T1E cells and CAR-EXO-T1E BioNVs. The degree of CRS (in vivo) will be assayed by measuring the serum levels of various cytokines (molecular IL-6, IL-2, and/or INF-v) and comparing them to the degree of cytokine release by whole cell controls. The concentration of mini-CAR NK GCP3 BioNVs, CAR NK GCP3 cells, and controls will be increased in a dose-dependent manner until CRS induction is observed.

Lymphocyte Recruitment and PD-L1 Blocking: mini-CAR BioNV targeting a check point inhibitor (CPI), such as PDL1 or CTLA4, (e.g., through a mono or bispecific CAR construct or transmembrane antibody), will be assayed for BioNV uptake and cell/tumor killing as described above. BioNVs with anti-CPI CARs will also be for natural lymphocyte recruitment and/or lymphocyte activity. Lymphocyte recruitment and/or activity to the tumor microenvironment (TME) will be evaluated via flow cytometry and/or measuring the concentrations of cytokines related to lymphocyte migration via PCR, ELIA, immunoblot, and/or NanoFCM.

Animal Models: one of more of the following animal models will be used:

SCID (CB17) or SCID (Beige) Models: use SCID mice with a CB17 or Beige background. These mice have a non-functional immune system.

NOD-SCID Model: NOD-SCID (non-obese diabetic SCID) mice have a combined immune deficiency and are commonly used for xenograft studies. They allow the engraftment of human lung cancer cells or tumor tissues.

NSG (NOD-SCID IL-2Rγnull) Models: NSG mice are highly immunodeficient and lack the IL-2Rγ receptor, making the mice permissive to human cell engraftment.

Rag2−/−γc−/− Model: Rag2−/−γc−/− mice are double knockout mice lacking the Rag2 gene and the common gamma chain (γc) gene.

NOG (NOD/Shi-scid/IL2Rγnull) Model: NOG mice are highly immunodeficient and lack both T cells and B cells.

The Xenograft Models listed below may be used (or tumor models adapted to chip—‘tumor on a chip’) to study the effects of mini-CAR BioNVs (either alone or as a co-treatment with whole cell CAR therapies) in vivo:

### Lung Cancer Tumors:

A549 Xenograft Model: human lung adenocarcinoma cell line used to establish xenograft models in mice.

H460 Xenograft Model: human lung carcinoma cell line used for establishing xenograft models in mice.

HCC827 Xenograft Model: human lung adenocarcinoma cell line harboring an epidermal growth factor receptor (EGFR) mutation.

NCI-H1975 Xenograft Model: human lung adenocarcinoma cell line harboring both EGFR and T790M mutations associated with resistance to EGFR-targeted therapies.

Patient-Derived Xenograft (PDX) Models: PDX models involve implanting patient-derived lung cancer tissues directly into mice. The model retains the characteristics and heterogeneity of the original patient tumors.

### Hepatocellular Carcinoma Tumors:

HepG2 and Huh7 Xenograft Models: human hepatocellular carcinoma cell lines used to establish xenograft models in mice.

PLC/PRF/5 Xenograft Model: human hepatocellular carcinoma cell line derived from a patient with liver cancer.

SK-HEP-1 Xenograft Model: human hepatocellular carcinoma cell line that is commonly used for studying HCC biology and investigating therapeutic approaches.

Patient-Derived Xenograft (PDX) Models: PDX models involve implanting patient-derived HCC tissues directly into mice. The model retains the characteristics and heterogeneity of the original patient tumors.

FRG (Fah−/−Rag2−/−II2rg−/−) Models: FRG mice are triple knockout mice lacking the fumarylacetoacetate hydrolase (Fah) gene, Rag2 gene, and II2rg gene. These mice have liver damage and are highly permissive for the engraftment of human HCC cells.

### Breast Cancer Tumors:

MCF-7, MDA-MB-231, and 4T1 Xenograft Models: human breast cancer cell lines used in xenograft models. Cells can be implanted into the mammary fat pads of nude mice, where they will grow and form tumors.

### Brain Cancer Tumors:

U87MG U251 Xenograft Model: human glioblastoma multiforme, an aggressive form of brain cancer.

GL261 Xenograft Model: murine glioblastoma used as a syngeneic mouse xenograft model.

D283 Med Xenograft Model: pediatric brain tumor called medulloblastoma.

BT-474 Xenograft Model: originally derived from a breast cancer patient, the BT-474 cell line is also used as a xenograft model for brain metastases to colonize the brain.

D283 Med: pediatric medulloblastoma.

Patient-Derived Xenograft (PDX) Models: PDX models involve implanting patient-derived lung cancer tissues directly into mice. The model retains the characteristics and heterogeneity of the original patient tumors.

### Renal Cell Carcinoma:

Renca Xenograft Model: murine renal adenocarcinoma cell (RCC) line derived from BALB/c mice.

ACHN, Caki-1, 786-O Xenograft Models: human RCC cell lines.

OS-RC-2 Xenograft Model: human RCC cell line derived from a patient with clear cell RCC.

### Lymphoma Tumors:

Raji and Ramos Xenograft Models: derived from a Burkitt's lymphoma patient used to study B-cell lymphomas.

SU-DHL-4 Xenograft Model: diffuse large B-cell lymphoma (DLBCL) cell line.

Granta-519 Xenograft Model: mantle cell lymphoma (MCL) cell line for MCL tumor growth, invasion, and response to therapies.

Pfeiffer Xenograft Model: derived from a primary effusion lymphoma (PEL) patient with unique characteristics, such as association with human herpesvirus 8 (HHV-8).

### Leukemia Tumors:

Subcutaneous Xenograft Models: human leukemia cells are injected subcutaneously into immunodeficient mice, typically SCID, NOD-SCID, NSG, or NOG mice.

Orthotopic Xenograft Models: Orthotopic models involve the injection of leukemia cells into the anatomically relevant site, such as the bone marrow or spleen, of immunodeficient mice.

Intravenous Xenograft Models: human leukemia cells are injected directly into the bloodstream of immunodeficient mice.

Patient-derived Xenograft (PDX) Models: PDX models involve the transplantation of patient-derived leukemia cells into immunodeficient mice. These models recapitulate the heterogeneity and characteristics of human leukemia in mice.

Genetic manipulation models: In addition to directly engrafting human leukemia cells, genetically engineered mouse models (GEMMs) can be used to develop leukemia.

### Melanoma Tumors:

Subcutaneous xenograft models: human melanoma cells are injected subcutaneously into immunodeficient mice, typically SCID, NOD-SCID, NSG, or NOG mice.

Orthotopic xenograft models: injection of melanoma cells into an anatomically relevant site, such as the skin or dermis, of immunodeficient mice. Th model mimics the microenvironment of melanoma and shows tumor invasion, angiogenesis, and interactions with the surrounding tissue.

Metastatic xenograft models: injection of melanoma cells directly into the bloodstream or via organ-specific injections, such as intravenous or intrasplenic injection, to promote metastatic spread.

Patient-derived xenograft (PDX) models: PDX models involve the transplantation of patient-derived melanoma cells or tumor tissue fragments into immunodeficient mice.

Genetic manipulation models: Genetically engineered mouse models (GEMMs) can be employed to develop melanoma with specific genetic alterations, such as activating mutations in genes like BRAF and/or NRAS, to initiate melanoma formation.

The cell lines may be used in combination or for controls. For example, in a mini-CAR EGFR BioNV therapeutic experiment targeting lung cancer, an EGFR+ve and an EGFR−ve model) to test for specificity.

Any cell lines and/or animal models described herein can be used for the evaluation of BioNVs and cell lines used to generate BioNVs, as well as evaluating controls thereof.

### Definitions

The following definitions are used in connection with the disclosure herein. Unless defined otherwise, all technical and scientific terms used herein have the same meaning as commonly understood to one of skill in the art to which this disclosure belongs.

An “effective amount,” or “therapeutically effective amount,” is an amount that is effective for treating, preventing, or ameliorating a cancer such as those described herein, or an amount that is intended to reduce the number of malignant cells, reduce a primary tumor volume, diminish angiogenesis, reduce the size and/or number of metastasis, or ameliorate a symptom associated with a cancer in a subject.

As used herein, “a,” “an,” or “the” can mean one or more than one.

As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present technology that do not contain those elements or features.

Although the open-ended term “comprising,” as a synonym of terms such as including, containing, or having, is used herein to describe and claim the present disclosure, or embodiments thereof, and may alternatively be described using alternative terms such as “consisting of” or “consisting essentially of.”

In embodiments, a “BioNV” refers to allogeneic, hypoimmunogenic, and biomimetic nanovesicles (NVs) which comprise at least one surface-oriented, membrane-embedded CAR. In embodiments, “nanovesicles (NVs),” as referred to herein, are lipid-bound vesicles on the order of about 10 nm to about 1200 nm in size which encapsulate an aqueous core. In embodiments, lipid-bound NVs can form using lipid monolayers, lipid bilayers, or maintain multilamellar forms. In embodiments, BioNV refers to biologically-derived nano-sized vesicles that can have designed biological functionalization. In embodiments, BioNVs are “biomimetic” in that they are derived from endogenous cellular material, more specifically, they substantially recapitulate plasma membrane material found in cells. In embodiments, the cells from which BioNVs originate can include stem cells of any kind, including cell types differentiated from said stem cells. In embodiments, BioNVs are substantially free of encapsulated cellular debris including cellular genomic nucleic acid, organelles, or organelle parts. In embodiments, BioNVs are characterized as having one or more, 2 or more, 3 or more, 4 or more, 5 or more, or 6 or more of the following:
a. being about 10 nm to about 1200 nm in size;
b. having a total volume of about 500 nm³ to about 5 μm³ (assuming spherical shape);
c. having a content of at least one phospholipid and cholesterol;
d. having a surface membrane having one or more of CD34, CCL21, PD-L1 (in BioNVs derived from non-activated cell sources), FasL, SerpinB9, H2-M3, CD47, CTLA-4, CD24, CD200, MFG-E8, NCAM, α-phagocytic integrin, and/or anti-6R antibody or antibody format, or a chimera of any one or more thereof; having a surface membrane substantially lacking T cell receptor components (TRAC and/or TRBC), MHC class I components, and/or MHC class II components, lacking proteins of one or more of HLA-A, HLA-B, HLA-C, HLA-E or HLA-G (but not both of HLA-E and HLA-G), HLA-F, and/or CIITA, SerpinB9, substantially lacking proteins inside the vesicle of one or more of IL-4, IL-6, IL-10, and/or IL-16;
e. having a membrane-embedded CAR comprising a target-binding moiety which can include an antibody or antibody format selected from one or more of a monoclonal antibody, polyclonal antibody, antibody fragment, Fab, Fab′, Fab′-SH, F(ab′)2, Fv, single chain Fv (scFv), diabody, nanobody, linear antibody, bispecific antibody, multi-specific antibody, chimeric antibody, humanized antibody, human antibody, fusion protein comprising the antigen-binding portion of an antibody, Bispecific T cell Engagers (BiTE), VERR/viral ligand, or by a variable heavy chain IgG fragment V_{H}H or V_{NAR} or engineered T-Cell Receptor (TCR), wherein the CAR can target a single biomarker, multiple biomarkers, or multiple parts of a single biomarker;
f. being capable of adsorbing and/or encapsulating a payload of one or more perforins, granzymes, cytokines, cytotoxic proteins, checkpoint inhibiting agents, gene editing payloads, antibodies or antibody fragments, fusion proteins, small molecules, biologics, radionuclides, tracing agents, dyes, fluorescent proteins, among other therapeutic payloads, and/or any combination thereof; and
g. being capable of not causing a deleterious immune reaction in subjects.

In embodiments, “induced pluripotent stem cells,” or “iPSCs” refers to stem cells that can be generated directly from adult cells. iPSCs can originate from differentiated cells that are reprogrammed back into an embryonic-like pluripotent state. iPSCs can generally propagate indefinitely and become any cell type of the organism they originate.

In embodiments, “allogeneic,” as used herein, refers to biological material, tissues, or cells, which are genetically dissimilar and originally immunological incompatible, despite originating from the same species. Allogeneic BioNVs, for example, are material that originates from a first subject (iPSC donor) and can be provided to any number of distinct subjects who are not genetically identical.

In embodiments, “hypoimmunogenic” or “hypoimmune,” as used herein in reference to a modified cell and/or BioNV, refers to a reduced capacity to generate an immunological response. In embodiments, iPSCs and BioNVs can be hypoimmunogenic due to reduced or ablated expression and/or activity of one or more immunogenic cell surface proteins and/or secreted proteins, such as TCR proteins, CRS proteins, MHC class I or MHC class II proteins, etc. In embodiments, iPSCs and BioNVs can be hypoimmunogenic due to expression of one or more immunoprotective cell surface proteins, such as CD47, CD34, CD24, CD200, α-phagocytic integrins, etc. In embodiments, BioNVs can be hypoimmunogenic due to not triggering CRS in a subject and/or not inducing HLA incompatibility.

In embodiments, “knocking-out,” “silencing,” “inactivating,” “disrupting,” or “blocking,” and their equivalencies, with respect to transcription, gene expression, or protein expression, refers to an amount of transcription, gene or protein expression which is reduced from a normal state or less than the wild-type state in a particular cell subset. The reduction can be significant such that no gene expression occurs, or a negligible amount of expression occurs.

In embodiments, “overexpression,” as used herein, refers to an amount of transcription, or gene or protein expression which is increased from a normal state or more than the wild-type state in a particular cell subset.

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

As used herein, all headings are simply for organization and are not intended to limit the disclosure in any manner. The content of any individual section may be equally applicable to all sections.

### INCORPORATION BY REFERENCE

All patents and publications referenced herein are hereby incorporated by reference in their entireties, including published PCT application, WO 2020/227369, filed May 6, 2020, titled “Tailored Hypoimmune Nanovesicle Delivery Systems for Cancer Tumors,” and published U.S. non-provisional application, US20220040106 A1, filed Aug. 3, 2021, titled “Tailored Hypoimmune Nanovesicular Delivery Systems for Cancer Tumors, Hereditary and Infectious Diseases.”

What is claimed is:

1. A method of treating or preventing a cancer comprising administering to a subject in need thereof:
(i) a therapeutically effective amount of a biomimetic nanovesicle (BioNV) comprising:
(a) a membrane-embedded chimeric antigen receptor (CAR) targeted against a cell surface marker; and
(b) a PD-1, PD-L1, and/or PD-L2 inhibiting agent; or
(ii) a therapeutically effective amount of a BioNV comprising a membrane-embedded CAR targeted against a cell surface marker, wherein the subject is undergoing treatment with a PD-1, PD-L1, and/or PD-L2 inhibiting agent.

2. A method of treating or preventing a cancer comprising administering to a subject in need thereof:
(i) a therapeutically effective amount of a biomimetic nanovesicle (BioNV) comprising:
(a) a membrane-embedded chimeric antigen receptor (CAR) targeted against a cell surface marker; and
(b) a conjugated and/or membrane-anchored PD-L1 and/or PD-L2 inhibiting agent; or
(ii) a therapeutically effective amount of a BioNV comprising:
(a) a membrane-embedded bispecific CAR targeted against a first cell surface marker and a second cell surface marker; and
(b) a conjugated and/or membrane-anchored PD-L1 and/or PD-L2 inhibiting agent.

3. A method of treating or preventing a cancer comprising administering to a subject in need thereof:
(i) a therapeutically effective amount of a biomimetic nanovesicle (BioNV) comprising a bispecific chimeric antigen receptor (CAR) targeted against a first cell surface marker and either PD-L1 or PD-L2, and wherein the first cell surface marker is not PD-1, PD-L1 or PD-L2; or
(ii) a therapeutically effective amount of a BioNV comprising
(a) a bispecific CAR targeted against the first cell surface marker and either PD-L1 or PD-L2, and wherein the first cell surface marker is not PD-1, PD-L1, or PD-L2; and
(b) a PD-1, PD-L1, and/or PD-L2 inhibiting agent; or
(iii) a therapeutically effective amount of a BioNV comprising a bispecific CAR targeted against either PD-L1 or PD-L2 and a first cell surface marker, wherein the subject is undergoing treatment with a PD-1, PD-L1, and/or PD-L2 inhibiting agent, and wherein the first cell surface marker is not PD-1, PD-L1, or PD-L2.

4. A method of treating or preventing a cancer comprising administering to a subject in need thereof a therapeutically effective amount of a biomimetic nanovesicle (BioNV) comprising
(a) a bispecific chimeric antigen receptor (CAR) targeted against a first cell surface marker and a second cell surface marker; and
(b) a PD-1, PD-L1, and/or PD-L2 inhibiting agent.

5. A method of treating or preventing a cancer comprising administering to a subject in need thereof a therapeutically effective amount of a biomimetic nanovesicle (BioNV) comprising a bispecific chimeric antigen receptor (CAR) targeted against a first cell surface marker and a second cell surface marker, wherein the subject is undergoing treatment with a PD-1, PD-L1, and/or PD-L2 inhibiting agent.

6. The method of any one of claims 2, 4, and 5, wherein the first cell surface marker and the second cell marker are selected from Table 1 and/or Table 2.

7. The method of claim 3, wherein the first cell surface marker selected from Table 1 and/or Table 2.

8. The method of any one of claims 2-7, wherein the BioNV binds at least a first cell and at least a second cell to initiate an anti-cancer response.

9. The method of claim 8, wherein the first cell is a cancer cell, and the second cell is an immune cell.

10. The method of any one of the preceding claims, wherein the PD-1 inhibiting agent is an antibody targeted against PD-1, optionally pembrolizumab, nivolumab, or cemiplimab.

11. The methods of any one of claims 1-9, wherein the PD-L1 and/or PD-L2 inhibiting agent is an antibody targeted against PD-L1 and/or PD-L2, optionally atezolizumab, avelumab, or durvalumab.

12. The method of any one the preceding claims, wherein the BioNV originates from a modified cell.

13. The method of claim 12, wherein the modified cell is a stem cell, an induced pluripotent stem cell (iPSC), a reprogrammed pluripotent or multipotent cell, an embryonic stem cell, a mesenchymal stem cell, or a differentiated cell which originates from any modified cell thereof.

14. The method of claim 13, wherein the modified cell is an iPSC.

15. The method of claim 12, wherein the modified cell is a T cell, helper T cell, T-memory cell, or NK cell.

16. The method of claim 12, wherein the modified cell is a macrophage.

17. The method of claim 12, wherein the modified cell is a monocyte.

18. The method of any one of claims 12-17, wherein the modified cell substantially lacks one or more MHC class I proteins, MHC class II proteins, T cell receptor (TCR) proteins, and/or cytokine release syndrome (CRS) proteins.

19. The method of any one of claims 12-17, wherein the modified cell has reduced or ablated expression of a β2-macroglobulin (B2M) gene and/or reduced, or ablated MHC class I protein expression and/or activity.

20. The method of any one of claims 12-19, wherein the modified cell has reduced or ablated expression of a CIITA gene and/or reduced, or ablated MHC class II protein expression and/or activity.

21. The method of any one of claims 12-20, wherein the modified cell has reduced or ablated expression of an HLA-A gene and/or reduced, or ablated HLA-A protein expression and/or activity.

22. The method of any one of claims 12-21, wherein the modified cell has reduced or ablated expression of an HLA-B gene and/or reduced, or ablated HLA-B protein expression and/or activity.

23. The method of any one of claims 12-22, wherein the modified cell has reduced or ablated expression of an HLA-C gene and/or reduced, or ablated HLA-C protein expression and/or activity.

24. The method of any one of claims 12-23, wherein the modified cell has reduced or ablated expression of an HLA-E or HLA-G gene and/or reduced, or ablated HLA-E or HLA-G protein expression and/or activity.

25. The method of any one of claims 12-24, wherein the modified cell has reduced or ablated expression of an HLA-F gene and/or reduced, or ablated HLA-F protein expression and/or activity.

26. The method of any one of claims 12-25, wherein the modified cell has reduced or ablated expression of a T cell alpha constant (TRAC) gene and/or reduced, or ablated TRAC protein expression and/or activity.

27. The method of any one of claims 12-26, wherein the modified cell has reduced or ablated expression of a T cell beta constant (TRBC) gene and/or reduced, or ablated TRBC protein expression and/or activity.

28. The method of any one of claims 12-27, wherein the modified cell has reduced or ablated expression of a PD-1 gene and/or reduced, or ablated PD-1 protein expression and/or activity.

29. The method of any one of claims 12-28, wherein the modified cell has reduced or ablated expression of an IL-4 gene and/or reduced, or ablated IL-4 protein expression and/or activity.

30. The method of any one of claims 12-29, wherein the modified cell has reduced or ablated expression of an IL-6 gene and/or reduced, or ablated IL-6 protein expression and/or activity.

31. The method of any one of claims 12-30, wherein the modified cell has reduced or ablated expression of an IL-10 gene and/or reduced, or ablated IL-10 protein expression and/or activity.

32. The method of any one of claims 12-31, wherein the modified cell has reduced or ablated expression of an IL-16 gene and/or reduced, or ablated IL-16 protein expression and/or activity.

33. The method of any one of claims 12-32, wherein the modified cell has reduced or ablated expression of a SerpinB9 gene and/or reduced, or ablated SerpinB9 protein expression and/or activity.

34. The method of any one of claims 12-33, wherein the modified cell expresses or has increased expression of a CD34 gene and/or gene product.

35. The method of any one of claims 12-34, wherein the modified cell expresses or has increased expression of a CCL2 gene and/or gene product.

36. The method of any one of claims 12-35, wherein the modified cell expresses or has increased expression of a PD-L1 gene and/or gene product, and wherein the modified cell is not activated; or wherein the modified cell has reduced or ablated expression of a PD-L1 gene and/or gene product, and wherein the modified cell is activated.

37. The method of any one of claims 12-36, wherein the modified cell expresses or has increased expression of a H2-M3 gene and/or gene product.

38. The method of any one of claims 12-37, wherein the modified cell expresses or has increased expression of a CD47 gene and/or gene product.

39. The method of any one of claims 12-38, wherein the modified cell expresses or has increased expression of a CD24 gene and/or gene product.

40. The method of any one of claims 12-39, wherein the modified cell expresses or has increased expression of a chimeric CD24/CD47 gene and/or gene product.

41. The method of any one of claims 12-40, wherein the modified cell expresses or has increased expression of a chimeric CD200 gene and/or gene product.

42. The method of any one of claims 12-41, wherein the modified cell expresses or has increased expression of a chimeric CD24/CD200 gene and/or gene product or a chimeric CD47/CD200 gene and/or gene product.

43. The method of any one of claims 12-42, wherein the modified cell expresses or has increased expression of a chimeric CTLA-4 gene and/or gene product.

44. The method of any one of claims 12-43, wherein the modified cell expresses or has increased expression of a chimeric MFG-E8 gene and/or gene product.

45. The method of any one of claims 12-44, wherein the modified cell expresses or has increased expression of an NCAM gene and/or gene product.

46. The method of any one of claims 12-45, wherein the modified cell expresses or has increased expression of a chimeric α-phagocytic integrin gene and/or gene product.

47. The method of any one of claims 12-46, wherein the modified cell expresses or has increased expression of an antibody or antibody format molecule which targets an IL-6 surface receptor (anti-IL-6R).

48. The method of any one of claims 12-46, wherein the modified cell expresses or has increased expression of a FasL gene and/or gene product.

49. The method of any one of claims 12-48, wherein the modified cell wherein does not overexpress a FasL gene and/or gene product.

50. The method of any one of claims 12-49, wherein the modified cell has reduced or ablated expression and/or activity of 3 or more immunogenic proteins, 4 or more immunogenic proteins, 5 or more immunogenic proteins, 6 or more immunogenic proteins, 7 or more immunogenic proteins, 8 or more immunogenic proteins, 9 or more immunogenic proteins, 10 or more immunogenic proteins, 11 or more immunogenic proteins, or 12 or more immunogenic proteins.

51. The method of any one of claims 12-50, wherein the modified cell expresses or has increased expression of 3 or more immunoprotective proteins, 4 or more immunoprotective proteins, 5 or more immunoprotective proteins, 6 or more immunoprotective proteins, 7 or more immunoprotective proteins, 8 or more immunoprotective proteins, 9 or more immunoprotective proteins, or 10 or more immunoprotective proteins.

52. The method of any one of claims 12-51, wherein the modified cell is allogeneic.

53. The method of any one of claims 12-52, wherein the modified cell does not cause an immune reaction in patients to which it or a BioNV derived therefrom is administered.

54. The method of any one of claims 12-53, wherein the modified cell is differentiated prior to BioNV formation.

55. The method of any one of claims 12-54, wherein the modified cell expresses the CAR under the control of a regulatable expression element.

56. The method of any one of the preceding claims, wherein the CAR is activated prior to BioNV formation.

57. The method of claim 56, wherein the CAR is activated via its target, through another receptor, and/or a virus.

58. The method of any one of claims 12-57, wherein the BioNV is formed from the modified cell, or the differentiated cell thereof, by sonication, adaptive focused acoustics technology, French press, extrusion, serial extrusion, cell lysis by detergent, and/or electroporation.

59. The method of claim 58, wherein the BioNV is formed from the modified cell by serial extrusion.

60. The method of any one of the preceding claims, wherein the BioNV is about 10 nm to about 1200 nm in size.

61. The method of any one of the preceding claims, wherein the BioNV is about 10 nm to about 100 nm in size.

62. The method of any one of claims 1-60, wherein the BioNV is about 100 nm to about 200 nm in size.

63. The method of any one of claims 1-60, wherein the BioNV is about 200 nm to about 500 nm in size.

64. The method of any one of claims 1-60, wherein the BioNV is about 500 nm to about 1200 nm in size.

65. The method of any one of the preceding claims, wherein the BioNV substantially lacks expression and/or activity of HLA-A, HLA-B, HLA-C, HLA-F, CIITA, IL-6, TRAC, TRBC, and one of either HLA-E or HLA-G.

66. The method of any one of the preceding claims, wherein the BioNV substantially lacks expression and/or activity of HLA-A, HLA-B, HLA-C, HLA-F, CIITA, IL-6, TRAC, TRBC, SerpinB9, and one of either HLA-E or HLA-G.

67. The method of any one of the preceding claims, wherein the BioNV substantially lacks expression and/or activity of HLA-A, HLA-B, HLA-C, HLA-F, CIITA, IL-6, TRAC, TRBC, SerpinB9, CD200, one of either HLA-E or HLA-G, and one or more of IL-4, IL-10, and IL-16.

68. The method of any one of the preceding claims, wherein the BioNV comprises a membrane-embedded α-phagocytic integrin, CCL2, H2-M3, FasL, MFG-E8, anti-IL-6R antibody or antibody format, and PD-L1 and/or CTLA-4; and
one of either CD24, CD47, CD200, chimeric CD24/CD47, chimeric CD24/CD200, and chimeric CD47/CD200, or two of either CD24, CD47, and CD200.

69. The method of any one of claims 1-67, wherein the BioNV comprises a membrane-embedded α-phagocytic integrin, CCL2, H2-M3, FasL, MFG-E8, anti-IL-6R antibody or antibody format, SerpinB9, and PD-L1 and/or CTLA-4; and
one of either CD24, CD47, CD200, chimeric CD24/CD47, chimeric CD24/CD200, and chimeric CD47/CD200, or two of either CD24, CD47, and CD200.

70. The method of any one of claims 1-67, wherein the BioNV comprises a membrane-embedded CD200 protein and substantially lacks either CD24 or CD47.

71. The method of any one of claims 1-67, wherein the BioNV substantially lacks protein and/or activity of SerpinB9 and CD200.

72. The method of any one of the preceding claims, wherein the CAR comprises an antibody or antibody format selected from one or more of a monoclonal antibody, polyclonal antibody, antibody fragment, V_{NAR}, V_{H}H, Fab, Fab′, Fab′-SH, F(ab′)2, Fv, single chain Fv (scFv), diabody, nanobody, linear antibody, bispecific antibody, multi-specific antibody, chimeric antibody, humanized antibody, human antibody, or fusion protein comprising the antigen-binding portion of an antibody.

73. The method of claim 71, wherein the antibody format is the scFv.

74. The method of any one of the preceding claims, wherein the CAR comprises a transmembrane domain derived from CD28, CD3ζ, CD4, CD8α, ICOS, or fragment and/or combination thereof.

75. The method of any one of the preceding claims, wherein the CAR comprises an intracellular domain further comprising an intracellular signaling domain of a CD3ζ-chain and/or one or more co-stimulatory molecules, optionally selected from CD28, 4-1BB, ICOS, CD27, and OX40.

76. The method of any one of the preceding claims, wherein the CAR is targeted against a cancer-specific antigen.

77. The method of any one of the preceding claims, wherein the BioNV, or the modified cell derived therefrom, comprises a nucleic acid encoding green fluorescence protein (GFP) and/or a GFP protein.

78. The method of claim 77, wherein the nucleic acid encoding GFP is operably linked to a promoter from one or more of IL-2, perforin, granzyme, alarmin, TNF, INF, and/or a combination thereof.

79. The method of any one of the preceding claims, wherein the BioNV encapsulates a payload.

80. The method of claim 79, wherein the payload is one or more of a gene editor, cytotoxic protein, biologic, nucleic acid, fusion protein, fluorescent protein, tracing dye, radionuclide, and/or small molecule.

81. The method of claim 79, wherein the payload is a therapeutic payload for a cancer type that the CAR is targeted against.

82. The method of claim 79, wherein the payload comprises an alkylating agent.

83. The method of claim 79, wherein the payload comprises an anthracycline.

84. The method of claim 79, wherein the payload comprises an antimetabolite.

85. The method of claim 79, wherein the payload comprises an anti-tumor antibiotic.

86. The method of claim 79, wherein the payload comprises an anti-tumor antibody or antibody format.

87. The method of claim 79, wherein the payload comprises a corticosteroid.

88. The method of claim 79, wherein the payload comprises a plant alkaloid.

89. The method of claim 79, wherein the payload comprises a topoisomerase inhibitor.

90. The method of claim 79, wherein the payload comprises a checkpoint inhibitor.

91. The method of claim 79, wherein the payload is selected from Abecma, Abemaciclib, Abiraterone Acetate, Abraxane, ABVD, ABVE, ABVE-PC, AC, Acalabrutinib, AC-T, Actemra, Adcetris, ADE, Ado-Trastuzumab Emtansine, Adriamycin, Afatinib Dimaleate, Afinitor, Akynzeo, Aldara, Aldesleukin, Alecensa, Alectinib, Alemtuzumab, Alimta, Aliqopa, Alkeran for Injection, Alkeran Tablets, Aloxi, Alpelisib, Alunbrig, Ameluz, Amifostine, Aminolevulinic Acid Hydrochloride, Amivantamab-vmjw, Anastrozole, Apalutamide, Aprepitant, Aranesp, Aredia, Arimidex, Aromasin, Arranon, Arsenic Trioxide, Arzerra, Asciminib Hydrochloride, Asparaginase Erwinia Chrysanthemi, Asparlas, Atezolizumab, Avapritinib, Avastin, Avelumab, Axicabtagene Ciloleucel, Axitinib, Ayvakit, Azacitidine, Azedra, Balversa, Bavencio, BEACOPP, Belantamab Mafodotin-blmf, Beleodaq, Belinostat, Belzutifan, Bendamustine Hydrochloride, Bendeka, BEP, Besponsa, Besremi, Bevacizumab, Bexarotene, Bicalutamide, BiCNU, Binimetinib, Blenrep, Bleomycin Sulfate, Blinatumomab, Blincyto, Bortezomib, Bosulif, Bosutinib, Braftovi, Brentuximab Vedotin, Brexucabtagene Autoleucel, Breyanzi, Brigatinib, Brukinsa, BuMel, Busulfan, Busulfex, Cabazitaxel, Cablivi, Cabometyx, Cabozantinib-S-Malate, CAF, Calaspargase Pegol-mknl, Calquence, Campath, Camptosar, Capecitabine, Caplacizumab-yhdp, Capmatinib Hydrochloride, CAPOX, Carac, Carboplatin, CARBOPLATIN-TAXOL, Carfilzomib, Carmustine, Carmustine Implant, Casodex, CEM, Cemiplimab-rwlc, Ceritinib, Cerubidine, Cervarix (Recombinant HPV Bivalent Vaccine), Cetuximab, CEV, Chlorambucil, CHLORAMBUCIL-PREDNISONE, CHOP, Cisplatin, Cladribine, Clofarabine, Clolar, CMF, Cobimetinib Fumarate, Cometriq, Copanlisib Hydrochloride, COPDAC, Copiktra, COPP, COPP-ABV, Cosmegen, Cotellic, Crizotinib, CVP, Cyclophosphamide, Cyramza, Cytarabine, Dabrafenib Mesylate, Dacarbazine, Dacogen, Dacomitinib, Dactinomycin, Danyelza, Daratumumab, Daratumumab and Hyaluronidase-fihj, Darbepoetin Alfa, Darolutamide, Darzalex, Darzalex Faspro, Dasatinib, Daunorubicin Hydrochloride, Daunorubicin Hydrochloride and Cytarabine Liposome, Daurismo, Decitabine, Decitabine and Cedazuridine, Defibrotide Sodium, Defitelio, Degarelix, Denileukin Diftitox, Denosumab, Dexamethasone, Dexrazoxane Hydrochloride, Dinutuximab, Docetaxel, Dostarlimab-gxly, Doxil, Doxorubicin Hydrochloride, Doxorubicin Hydrochloride Liposome, Durvalumab, Duvelisib, Efudex, Eligard, Elitek, Ellence, Elotuzumab, Eloxatin, Eltrombopag Olamine, Elzonris, Emapalumab-Izsg, Emend, Empliciti, Enasidenib Mesylate, Encorafenib, Enfortumab Vedotin-ejfv, Enhertu, Entrectinib, Enzalutamide, Epirubicin Hydrochloride, EPOCH, Epoetin Alfa, Epogen, Erbitux, Erdafitinib, Eribulin Mesylate, Erivedge, Erleada, Erlotinib Hydrochloride, Erwinaze, Ethyol, Etopophos, Etoposide, Etoposide Phosphate, Everolimus, Evista, Evomela, Exemestane, Exkivity, 5-FU, 5-FU, Fam-Trastuzumab Deruxtecan-nxki, Fareston, Faslodex, FEC, Fedratinib Hydrochloride, Femara, Filgrastim, Firmagon, Fludarabine Phosphate, Fluoroplex, Fluorouracil Injection, Fluorouracil-Topical, Flutamide, FOLFIRI, FOLFIRI-BEVACIZUMAB, FOLFIRI-CETUXIMAB, FOLFIRINOX, FOLFOX, Folotyn, Fostamatinib Disodium, Fotivda, Fulphila, FU-LV, Fulvestrant, Fyarro, Gamifant, Gardasil (Recombinant HPV Quadrivalent Vaccine), Gardasil 9 (Recombinant HPV Nonavalent Vaccine), Gavreto, Gazyva, Gefitinib, Gemcitabine Hydrochloride, GEMCITABINE-CISPLATIN, GEMCITABINE-OXALIPLATIN, Gemtuzumab Ozogamicin, Gemzar, Gilotrif, Gilteritinib Fumarate, Glasdegib Maleate, Gleevec, Gliadel Wafer, Glucarpidase, Goserelin Acetate, Granisetron, Granisetron Hydrochloride, Granix, Halaven, Hemangeol, Herceptin Hylecta, Herceptin, HPV Bivalent Vaccine, Recombinant, HPV Nonavalent Vaccine, Recombinant, HPV Quadrivalent Vaccine, Recombinant, Hycamtin, Hydrea, Hydroxyurea, Hyper-CVAD, Ibrance, Ibritumomab Tiuxetan, Ibrutinib, ICE, Iclusig, Idamycin PFS, Idarubicin Hydrochloride, Idecabtagene Vicleucel, Idelalisib, Idhifa, Ifex, Ifosfamide, Interleukin-2 (recombinant), Imatinib Mesylate, Imbruvica, Imfinzi, Imiquimod, Imlygic, Infigratinib Phosphate, Infugem, Inlyta, Inotuzumab Ozogamicin, Inqovi, Inrebic, Interferon Alfa-2b, Intron A, Iobenguane I131, Ipilimumab, Iressa, Irinotecan Hydrochloride, Isatuximab-irfc, Istodax, Ivosidenib, Ixabepilone, Ixazomib Citrate, Ixempra, Jakafi, JEB, Jelmyto, Jemperli, Jevtana, Kadcyla, Kepivance, Keytruda, Kimmtrak, Kisqali, Koselugo, Kymriah, Kyprolis, Lanreotide Acetate, Lapatinib Ditosylate, Larotrectinib Sulfate, Lenalidomide, Lenvatinib Mesylate, Lenvima, Letrozole, Leucovorin Calcium, Leukeran, Leuprolide Acetate, Levulan Kerastik, Libtayo, Lisocabtagene Maraleucel, Lomustine, Loncastuximab Tesirine-lpyl, Lonsurf, Lorbrena, Lorlatinib, Lumakras, Lumoxiti, Lupron Depot, Lurbinectedin, Luspatercept-aamt, Lutathera, Lutetium (Lu 177-Dotatate), Lynparza, Margenza, Margetuximab-cmkb, Marqibo, Matulane, Mechlorethamine Hydrochloride, Megestrol Acetate, Mekinist, Mektovi, Melphalan, Melphalan Hydrochloride, Mercaptopurine, Mesna, Mesnex, Methotrexate Sodium, Methylnaltrexone Bromide, Midostaurin, Mitomycin, Mitoxantrone Hydrochloride, Mobocertinib Succinate, Mogamulizumab-kpkc, Monjuvi, MOPP, Moxetumomab Pasudotox-tdfk, Mozobil, MVAC, Mvasi, Myleran, Mylotarg, Nanoparticle Paclitaxel, Naxitamab-gqgk, Necitumumab, Nelarabine, Neratinib Maleate, Nerlynx, Netupitant, Neulasta, Neupogen, Nexavar, Nilandron, Nilotinib, Nilutamide, Ninlaro, Niraparib Tosylate Monohydrate, Nivestym, Nivolumab, Nplate, Nubeqa, Nyvepria, Obinutuzumab, Odomzo, OEPA, Ofatumumab, OFF, Olaparib, Omacetaxine Mepesuccinate, Oncaspar, Ondansetron Hydrochloride, Onivyde, Ontak, Onureg, Opdivo, OPPA, Orgovyx, Osimertinib Mesylate, Oxaliplatin, Paclitaxel, Paclitaxel Albumin-stabilized Nanoparticle Formulation, PAD, Padcev, Palbociclib, Palifermin, Palonosetron Hydrochloride, Pamidronate Disodium, Panitumumab, Paraplatin, Pazopanib Hydrochloride, PCV, PEB, Pegaspargase, Pegfilgrastim, Peginterferon Alfa-2b, PEG-Intron, Pemazyre, Pembrolizumab, Pemetrexed Disodium, Pemigatinib, Perjeta, Pertuzumab, Pexidartinib Hydrochloride, Phesgo, Piqray, Plerixafor, Polatuzumab Vedotin-piiq, Polivy, Pomalidomide, Pomalyst, Ponatinib Hydrochloride, Portrazza, Poteligeo, Pralatrexate, Pralsetinib, Prednisone, Procarbazine Hydrochloride, Procrit, Proleukin, Prolia, Promacta, Propranolol Hydrochloride, Provenge, Purinethol, Purixan, Qinlock, Radium 223 Dichloride, Raloxifene Hydrochloride, Ramucirumab, Rasburicase, Ravulizumab-cwvz, Reblozyl, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor, Relugolix, R-EPOCH, Retacrit, Retevmo, Revlimid, Ribociclib, R-ICE, Ripretinib, Rituxan, Rituxan Hycela, Rituximab, Rolapitant Hydrochloride, Romidepsin, Romiplostim, Ropeginterferon Alfa-2b-njft, Rozlytrek, Rubidomycin, Rubraca, Rucaparib Camsylate, Ruxolitinib Phosphate, Rybrevant, Rydapt, Rylaze, Sacituzumab Govitecan-hziy, Sancuso, Sarclisa, Sclerosol Intrapleural Aerosol, Selinexor, Selpercatinib, Selumetinib Sulfate, Scemblix, Siltuximab, Sipuleucel-T, Sirolimus Protein-Bound Particles, Soltamox, Somatuline Depot, Sonidegib, Sorafenib Tosylate, Sotorasib, Sprycel, STANFORD Sterile Talc Powder, Steritalc, Stivarga, Sunitinib Malate, Sustol, Sutent, Sylatron, Sylvant, Synribo, Tabloid, Tabrecta, TAC, Tafasitamab-cxix, Tafinlar, Tagraxofusp-erzs, Tagrisso, Talazoparib Tosylate, Talimogene Laherparepvec, Talzenna, Tamoxifen Citrate, Tarceva, Targretin, Tasigna, Tavalisse, Taxotere, Tazemetostat Hydrobromide, Tazverik, Tebentafusp-tebn, Tecartus, Tecentriq, Temodar, Temozolomide, Temsirolimus, Tepadina, Tepmetko, Tepotinib Hydrochloride, Thalidomide, Thalomid, Thioguanine, Thiotepa, Tibsovo, Tisagenlecleucel, Tisotumab Vedotin-tftv, Tivdak, Tivozanib Hydrochloride, Tocilizumab, Tolak, Topotecan Hydrochloride, Toremifene, Torisel, Totect, TPF, Trabectedin, Trametinib Dimethyl Sulfoxide, Trastuzumab, Trastuzumab and Hyaluronidase-oysk, Treanda, Trexall, Trifluridine and Tipiracil Hydrochloride, Trisenox, Trodelvy, Truseltiq, Truxima, Tucatinib, Tukysa, Turalio, Tykerb, Ukoniq, Ultomiris, Umbralisib Tosylate, Undencyca, Unituxin, Uridine Triacetate, VAC, Valrubicin, Valstar, Vandetanib, VAMP, Varubi, Vectibix, VelP, Velcade, Vemurafenib, Venclexta, Venetoclax, Verzenio, Vidaza, Vinblastine Sulfate, Vincristine Sulfate, Vincristine Sulfate Liposome, Vinorelbine Tartrate, VIP, Vismodegib, Vistogard, Vitrakvi, Vizimpro, Voraxaze, Vorinostat, Votrient, Vyxeos, Welireg, Xalkori, Xatmep, Xeloda, XELIRI, XELOX, Xgeva, Xofigo, Xospata, Xpovio, Xtandi, Yervoy, Yescarta, Yondelis, Yonsa, Zaltrap, Zanubrutinib, Zarxio, Zejula, Zelboraf, Zepzelca, Zevalin, Ziextenzo, Zinecard, Zirabev (Bevcizumab), Ziv-Aflibercept, Zofran, Zoladex, Zoledronic Acid, Zolinza, Zometa, Zyclara, Zydelig, Zykadia, Zynlonta, and Zytiga.

92. The method of claim 79, wherein the payload is one or more of pidilizumab, BMS-936559, tremelimumab, AGEN1884, and/or RG2077.

93. The method of claim 80, wherein the nucleic acid molecule encodes one or more of a CRISPR/Cas component, guide RNA (gRNA), tracer RNA (tracrRNA), micro RNA (miRNA), RNA inference (RNAi), small interference RNA (siRNA), duplex RNA, Piwi-interacting RNA (piRNA), small nuclear RNA (snRNA), small nucleolar RNA (snoRNA), antisense oligonucleotide (ASO), locked nucleic acid (LNA), splice switching oligonucleotide (SSO), tRNA, ribosomal RNA (rRNA), short hairpin (shRNA), complementary messenger RNA, repeat associated small interfering RNA (rasiRNA), and/or small non-coding RNA.

94. The method of claim 80, wherein the gene editing payload is one or more of a TALEN, ZFN, RNase P RNA, C2c1, C2c2, C2c3, Cas9, Cpf1, TevCas9, Archaea Cas9, CasY.1, CasY.2, CasY.3, CasY.4, CasY.5, CasY.6, CasX Cas omega, transposase, and/or any ortholog or homolog thereof.

95. The method of any one of the preceding claims, wherein the BioNV encapsulates one or more perforin molecules.

96. The method of any one of the preceding claims, wherein the BioNV encapsulates one or more granzyme molecules.

97. The method of claim 96, wherein the granzyme molecules are selected from granzyme A, B, H, K, and M.

98. The method of any one of the preceding claims, wherein the BioNV encapsulates one or more perforin molecules and/or one or more granzyme molecules derived from a cell from which the BioNV is derived.

99. The method of any one of claims 1-97, wherein the BioNV encapsulates one or more perforin molecules and/or one or more granzyme molecules exogenously added to the BioNV.

100. The method of any one of the preceding claims, further comprising co-administering a whole cell therapy.

101. The method of any one of the preceding claims, further comprising administering an additional therapeutic agent.

102. The method of any one of the preceding claims, wherein the BioNV is stored at about −80° C. or is suitable for storage at about −80° C.

103. The method of any one of the preceding claims, wherein the BioNV is lyophilized.

104. The method of any one of claim 2 or 6-103, wherein the PD-1 inhibiting agent is an antibody targeted against PD-1, optionally pembrolizumab, nivolumab, or cemiplimab.

105. The method of any one of claim 2 or 6-103, wherein the PD-L1 or PD-L2 inhibiting agent is an antibody targeted against PD-L1 or PD-L2, optionally atezolizumab, avelumab, or durvalumab, or the antigen-binding domain thereof, and is conjugated to the surface of the BioNV.

106. The method of any one of claim 2 or 6-103, wherein the PD-1 inhibiting agent is an antibody targeted against PD-1, optionally pembrolizumab or cemiplimab.

107. The method of any one of claim 2 or 6-103, wherein the PD-L1 or PD-L2 inhibiting agent is atezolizumab, avelumab, or durvalumab, or the antigen-binding domain thereof, and is membrane-anchored via a transmembrane domain fusion.

108. The method of any one of the preceding claims, wherein the cancer is a carcinoma.

109. The method of any one of claims 1-107, wherein the cancer is a sarcoma.

110. The method of any one of claims 1-107, wherein the cancer is a myeloma.

111. The method of any one of claims 1-107, wherein the cancer is a leukemia.

112. The method of any one of claims 1-107, wherein the cancer is a lymphoma.

113. The method of any one of claims 1-107, wherein the cancer is a mixed type cancer.

114. The method of any one of the preceding claims, wherein the cancer is metastatic.

115. The method of any one of claims 1-107, wherein the cancer is selected from Acute Biphenotypic Leukemia, Acute Eosinophilic Leukemia, Acute Lymphoblastic Leukemia, Acute Lymphocytic Leukemia, Acute Myeloid Dendritic Cell Leukemia, Acute Myeloid Leukemia, Adenocarcinoma of the Lung, Adrenal Gland Tumors, Adrenocortical Carcinoma, AIDS-related Cancers, AIDS-related Lymphoma, Alveolar Soft Part and Cardiac Sarcoma, Amyloidosis, Anal Cancer, Anaplastic Large Cell Lymphoma, Angioimmunoblastic T-cell Lymphoma, Appendix Cancer, Astrocytoma, Ataxia-telangiectasia, Attenuated Familial Adenomatous Polyposis, B-cell Prolymphocytic Leukemia, Basal Cell Carcinoma, Beckwith-Wiedemann Syndrome, Bile Duct Cancer, Birt-Hogg-Dubé Syndrome, Bladder Cancer, Bone Cancer, Brain and Nervous System Cancer, Brain Stem Glioma, Brainstem Glioma, Brain Tumors, Breast Cancer, Bronchial Adenomas/Carcinoids, Burkitt's Lymphoma, Carcinoid Tumor, Carcinoid Tumors, Carney Complex, Central Nervous System Tumors, Cerebellar Astrocytoma, Cerebral Astrocytoma, Cervical Cancer, Childhood Desmoplastic Ganglioglioma, Cholangiocarcinoma, Chondrosarcoma, Chronic Lymphocytic Leukemia, Chronic Myelogenous Leukemia, Chronic Myeloid Leukemia, Chronic T-cell Lymphocytic Leukemia, Colon Cancer, Colorectal Cancer, Cowden Syndrome, Craniopharyngioma, Cutaneous T-cell Lymphoma, Dermatofibrosarcoma Protuberans, Desmoplastic Small Round Cell Tumor, Diffuse Gastric Cancer, Diffuse Large B-cell Lymphoma, Endocrine System Cancer, Endocrine Tumors, Endometrial Cancer, Eosinophilic Leukemia, Ependymoma, Epithelioid Hemangioendothelioma (EHE), Esophageal Cancer, Ewing Sarcoma, Extragonadal Germ Cell Tumor, Extrahepatic Bile Duct Cancer, Eye Cancer, Eyelid Cancer, Fallopian Tube Cancer, Familial Adenomatous Polyposis, Familial Malignant Melanoma, Familial Clear Cell Renal Cell Carcinoma (RCC), Follicular Lymphoma, Gallbladder Cancer, Gardner Syndrome, Gastric (stomach) Cancer, Gastrointestinal Cancer, Gastrointestinal Carcinoid Tumor, Gastrointestinal Stromal Tumor, Gastrointestinal Stromal Tumor (GIST), Genitourinary and Gynecologic Cancer, Germ Cell Tumor, Gestational Trophoblastic Disease, Gestational Trophoblastic Tumor, Glioblastoma, Glioma, Hairy Cell Leukemia, Head and Neck Cancer, Hematopoietic Cancer, Hepatocellular Cancer, Hepatosplenic T-cell Lymphoma, HIV-related Cancer, Hodgkin's Lymphoma, Hypopharyngeal Cancer, Inflammatory Breast Cancer, Intravascular Large B-cell Lymphoma, Invasive Cribriform Carcinoma, Invasive Lobular Carcinoma, Islet Cell Carcinoma (endocrine Pancreas), Islet Cell Tumors, Juvenile Polyposis Syndrome, Kaposi Sarcoma, Keratoacanthoma, Kidney Cancer, Lacrimal Gland Tumor, Large Granular Lymphocytic Leukemia, Laryngeal and Hypopharyngeal Cancer, Leiomyomatosis and Renal Cell Cancer, Leiomyosarcoma, Li-Fraumeni Syndrome (LFS), Liposarcoma, Liver Cancer, Lung Cancer, Lymphomas of Primary Cutaneous Origin, Lymphomatoid Granulomatosis, Lymphoplasmacytic Lymphoma, Lynch Syndrome, Malignant Fibrous Histiocytoma of Bone, Mantle Cell Lymphoma, Marginal Zone B-cell Lymphoma, Mast Cell Leukemia, Mastocytosis, Mediastinal Large B Cell Lymphoma, Medullary Carcinoma, Medulloblastoma, Melanoma, Meningioma, Merkel Cell Carcinoma, Mesothelioma, Mixed Polyposis Syndrome, Mucosa-associated Lymphoid Tissue Lymphoma, Muir-Torre Syndrome (MTS), Multiple Endocrine Neoplasia Syndrome, Multiple Endocrine Neoplasia Type 1, Multiple Endocrine Neoplasia Type 2, Multiple Myeloma, Multiple Myeloma/Plasma Cell Neoplasm, Mycosis Fungoides, Myelodysplastic Syndromes, MYH-associated Polyposis, Myxosarcoma, Nasal Cavity and Paranasal Sinus Cancer, Nephroblastoma, Nasopharyngeal Cancer, Nasopharyngeal Carcinoma, Neuroblastoma, Neuroendocrine Tumors, Neurofibromatosis Type 1, Neurofibromatosis Type 2, Nevoid Basal Cell Carcinoma Syndrome, Nodal Marginal Zone B Cell Lymphoma, Non-Hodgkin Lymphoma, Non-small Cell Lung Cancer, Non-small Cell Lung Carcinoma, Oligodendroglioma, Optic Nerve Glioma, Oral and/or Oropharyngeal Cancer, Osteosarcoma, Ovarian Cancer, Ovarian Epithelial Cancer, Ovarian Germ Cell Tumor, Pancreatic Cancer, Papillary Renal Cell Carcinoma, Paranasal Sinus and Nasal Cavity Cancer, Parathyroid Cancer, Pelvic Cancer, Penile Cancer, Peutz-Jeghers Syndrome, Pharyngeal Cancer, Pheochromocytoma, Phyllodes Tumor, Pilocytic Astrocytoma, Pineal Astrocytoma, Pituitary Adenoma, Pituitary Gland Tumors, Plasmablastic Lymphoma, Pleuropulmonary Blastoma, Precursor B Lymphoblastic Leukemia, Primary Central Nervous System Lymphoma, Primary Cutaneous Follicular Lymphoma, Primary Cutaneous Immunocytoma, Primary Effusion Lymphoma, Primitive Neuroectodermal Tumor, Prostate Cancer, Rectal Cancer, Renal Cancer, Renal Cell Carcinoma, Retinoblastoma, Rhabdomyosarcoma, Salivary Gland Cancer, Sarcoma, Sarcomas of Primary Cutaneous Origin, Sebaceous Carcinoma, Sezary Syndrome, Skin Adnexal Tumors, Skin Cancer, Small Bowel Cancer, Small Cell Lung Cancer, Small Intestine Cancer, Splenic Marginal Zone Lymphoma, Squamous-cell Carcinoma of the Lung, Squamous Cell Carcinoma, Squamous Cell Skin Cancer, Stomach Cancer, Surface Epithelial-Stromal Tumor, T-cell Prolymphocytic Leukemia, Testicular Cancer, Thoracic and Respiratory Cancer, Thymoma, Thymic Carcinoma, Thyroid Cancer, Transitional Cell Cancer, Transitional Cell Canceradenoid Cystic Carcinoma, Tuberous Sclerosis Syndrome, Tubular Carcinoma, Turcot Syndrome, Unknown Primary Cancer, Unsorted Cancer, Ureter Cancer, Urethral Cancer, Uterine Cancer, Uterine Sarcoma, Uveal Melanoma, Vaginal Cancer, Visual Pathway and Hypothalamic Glioma, Von Hippel-Lindau (VHL) Syndrome, Vulvar Cancer, Wilms Tumor, and Xeroderma Pigmentosum.

116. An allogeneic, biomimetic nanovesicle (BioNV) comprising:
(a) a membrane-embedded chimeric antigen receptor (CAR) targeted against a cell surface marker; and
(b) a conjugated and/or membrane-anchored targeting agent targeted against PD-L1, or PD-L2,
wherein the agent targeted against PD-L1 or PD-L2 is atezolizumab, avelumab, or durvalumab, and
wherein the CAR is not targeted against PD-L1 or PD-L2.

117. An allogeneic, biomimetic nanovesicle (BioNV) comprising:
(a) a first membrane-embedded chimeric antigen receptor (CAR) targeted against a cell surface wherein the first membrane-embedded CAR is not targeted against PD-L1 or PD-L2; and
(b) at least a second membrane-embedded CAR targeted against PD-L1 or PD-L2.

118. An allogeneic, biomimetic nanovesicle (BioNV) comprising a bispecific, membrane-embedded chimeric antigen receptor (CAR) targeted against:
(a) a cell surface marker of a cancer cell; and
(b) PD-L1 or PD-L2, and
wherein the cell surface marker of the cancer cell is not PD-L1 or PD-L2.
